(19) United States
(12) Patent Application Publication
(10) Pub. No.: US 2024/0342294 A1
(43) Pub. Date: Oct. 17, 2024

(54) POLYMERS CONTAINING BETA-AMINO-ESTER (BAE) AND BETA-THIO-ESTER (BTE)

(71) Applicant: GEORGIA TECH RESEARCH CORPORATION, Atlanta, GA (US)

(72) Inventors: M.G. Finn, Atlanta, GA (US); Philip J. Santangelo, Smyrna, GA (US); Daryll Vanover, Atlanta, GA (US); Nicholas Bruno, Atlanta, GA (US)

(73) Assignee: GEORGIA TECH REAEARCH GROUP, Atlanta, GA (US)

(21) Appl. No.: 18/738,623

(22) Filed: Jun. 10, 2024

Related U.S. Application Data
(63) Continuation of application No. PCT/US2022/081249, filed on Dec. 9, 2022.
(60) Provisional application No. 63/287,691, filed on Dec. 9, 2021.

(51) Int. Cl. A61K 47/59; A61K 9/00; A61K 47/69; A61K 48/00; C08G 75/045

(52) U.S. Cl. CPC A61K 47/59; A61K 9/0078; A61K 47/6935; A61K 48/0041; C08G 75/045

(57) ABSTRACT

Provided herein are branched BAE/BTE-containing polymers useful as vehicles for the delivery of therapeutic agents, such as nucleic acids. The polymers form stable compositions and are suitable for the delivery of therapeutic agents via nebulization. Compositions of the disclosed polymers are capable of delivering therapeutic agents such as mRNA to lung epithelial cells.

### CROSS-REFERENCE TO RELATED APPLICATION

The present application claims the benefit of priority to U.S. Provisional Application No. 63/287,691, filed Dec. 9, 2021, the entire contents of which are hereby incorporated by reference.

### STATEMENT OF GOVERNMENT SUPPORT

This invention was made with government support under HR0011-19-2-0008, and HR0011-16-2-0016 awarded by the Defense Advanced Research Projects Agency. The government has certain rights in the invention.

### TECHNICAL FIELD

The disclosure is directed to polymers containing beta-amino-ester and beta-thio-ester groups (denoted BAE/BTE-containing polymers) as well as methods of making and using these materials by themselves or in combination.

### BACKGROUND

Nucleic acid-based therapeutics hold the potential to treat any disease with a protein target. DNA has been used for the majority of gene therapy clinical trials. The use of mRNA instead of DNA would mitigate the risk of insertional mutagenesis and also confer the ability to transfect non-dividing cells which would be an advantage, particularly in respiratory epithelium which is slowly dividing or terminally differentiated. For example, the ability of in vitro transcribed (IVT)-mRNA to express neutralizing antibodies against the viral pathogen, RSV, has been demonstrated in the lung, albeit using invasive intra-trachel delivery. The development of effective delivery systems for IVT-mRNA remain a critical hurdle for clinical adoption.

Branched polyethylenimine (bPEI) are known to be efficient delivery vectors for nebulized gene delivery. However, toxicity concerns related to bPEI remain, due to accumulation of the relatively large, non-degradable polymer. Lower molecular weight PEIs tend to have lower toxicity. However, DNA transfection efficiency is generally diminished with lower molecular weight PEIs, and those with molecular weights below approximately 1.8 kDa are ineffective. As such, there remains a need for effective, non-invasive vehicles for the delivery of therapeutic agents, such as nucleic acids. In particular, there remains a need for delivery vehicles that form stable compositions, suitable for the delivery of therapeutic agents, such as mRNA, via nebulization.

### SUMMARY

The present disclosure relates to linear and branched polymers containing beta-amino-ester and beta-thio-ester units in their main chains (BAE/BTE-containing polymers) useful for the non-viral delivery of agents (e.g., nucleic acids) to cells. The BAE/BTE-containing polymers of Formula (I), salts thereof, and embodiments described herein, are collectively referred to as “polymers of the invention.” The polymers of Formula (I) are linear, branched or hyperbranched. The degree of branching can be used to optimize properties such as solubility, viscosity, and efficacy as a transfection reagent. Polymers of the invention can be used to prepare stable formulations (e.g., particles) for nebulization or acrosol delivery.

In one aspect, provided herein are BAE/BTE-containing polymers of Formula (I), comprising: a backbone of Formula (A), an optional linking group of Formula (B), an optional branching group of Formula (C), and an optional end-capping group of Formula (D).

In another aspect, provided herein are compositions comprising a polymer of Formula (I) and an agent. In certain embodiments, the agent is a protein, peptide, polynucleotide, lipids or a small molecule.

Provided in other aspects are methods of delivering a polynucleotide to a cell by contacting the cell with a composition comprising a polymer of Formula (I) and methods of treating a disease or disorder in a subject in need of such treatment by administering to the subject a pharmaceutical composition comprising a polymer of Formula (I).

### BRIEF DESCRIPTION OF THE DRAWINGS

FIGS. 1A-1C depict a schematic for functional screening of polymers for nebulized mRNA delivery. FIG. 1A depicts a representative polymer showing exemplary components. FIG. 1B depicts one or more cargo RNAs formulates into polyplexes approximately 100-200 nm in diameter. This colloidal mixture is nebulized, creating droplets of about 4-6 mm, and inhaled. FIG. 1C depicts a polymer screening where polyplexes are nebulized into mice and lungs are isolated and analyzed by luminescence.

FIG. 2 depicts a microscopic image of hamster lung with mRNA (white spots) delivered via nebulization at 30 μg RNA per animal.

FIG. 3 depicts a microscopic image of hamster lung with RNA granules of a combination of aNLuc and aVHH mRNA (white spots) delivered via nebulization at 30 μg RNA per animal.

FIGS. 4A-4B depict luminescence imaging of lung tissue with aHCA-NLuc (GPI-anchored IgG heavy chain and light chain fused to a nano luciferase protein) mRNA delivered via nebulization at various doses. FIG. 4A depicts luminescence imaging of mice lung tissue with aHCA-NLuc mRNA delivered via nebulization by the indicated polymer at the indicated dose (ug RNA per animal). FIG. 4B depicts luminescence imaging of hamster lung tissue with aHCA-NLuc mRNA delivered via nebulization by the indicated polymer at the indicated dose (ug RNA per animal).

FIG. 5 depicts a chart quantifying fold change total flux of bioluminescence in mice lungs 24 hours after delivery of mRNA encoded aNLuc using the hDD90-118 PBAE at the indicated doses using nose-only nebulization.

FIG. 6 depicts a chart quantifying fold change total flux of bioluminescence in mice lungs 24 hours after delivery of mRNA encoded aNLuc using the indicated polymers at the indicated doses using nose-only nebulization.

FIGS. 7A-7B depict charts comparing polymers in mouse lungs treated with mRNA encoding aNLue using the indicated polymers at 5 kg/mg doses using nebulization. FIG. 7A depicts a chart quantifying average radiance of bioluminescence in mice 24 hours lungs after delivery of the indicated polymers at a 5 kg/mg dose. FIG. 7B depicts a chart quantifying mean intensity of aNLuc-positive RNA granules in mice lungs 4 hours lungs after delivery of the indicated polymers at a 5 kg/mg dose.

FIG. 8 depicts a chart of fold change total flux vs. Cas13a-NLuc mRNA dosage (μg) in hamster lung tissue with Cas13a-NLuc mRNA and guide RNAs delivered via nebulization using the indicated polymer.

FIGS. 9A-9I depict a range of mRNAs delivered by polymer 76 via nebulization in different mouse strains 24 hours post transfection. FIG. 9A depicts a luminescent image of DBA/2 and BALB/c mouse lungs treated with different doses of aHCA-NLuc mRNA formulated with polymer 76. FIG. 9B depicts a quantification of the fold change total flux of DBA/2 and BALB/c mouse lungs treated with different doses of aHCA-NLuc mRNA formulated with polymer 76. FIG. 9C depicts a quantification of weight loss of DBA/2 and BALB/c mice treated with different doses of aHCA-NLuc mRNA formulated with polymer 76. FIG. 9D depicts a luminescent image of DBA/2 and BALB/c mouse lungs treated with different doses of Cas13a-NLuc mRNA formulated with polymer 76. FIG. 9E depicts a quantification of the fold change total flux of DBA/2 and BALB/c mouse lungs treated with different doses of Cas13a-NLuc mRNA formulated with polymer 76. FIG. 9F depicts a quantification of weight loss of DBA/2 and BALB/c mice treated with different doses of Cas13a-NLuc mRNA formulated with polymer 76. FIG. 9G depicts a luminescent image of DBA/2 and BALB/c mouse lungs treated with different doses of dCas9-VPR-NLuc mRNA formulated with polymer 76. FIG. 9H depicts a quantification of the fold change total flux of DBA/2 and BALB/c mouse lungs treated with different doses of dCas9-VPR-NLuc mRNA formulated with polymer 76. FIG. 9I depicts a quantification of weight loss of DBA/2 and BALB/c mice treated with different doses of dCas9-VPR-NLuc mRNA formulated with polymer 76.

FIGS. 10A-10B depict ferret lung tissue with aHCA-NLuc (GPI-anchored IgG heavy chain and light chain fused to a nanoluciferase protein) mRNA delivered via nebulization at the indicated dosage. FIG. 10A depicts luminescence imaging of ferret lung tissue with aHCA-NLuc mRNA delivered via nebulization at the indicated dosage. FIG. 10B quantifies radiance of ferret lung tissue from luminescence imaging with aHCA-NLuc mRNA delivered via nebulization at the indicated dosage.

FIGS. 11A-11B depict ferret lung tissue with Cas13a-NLuc (Cas13a fused to a nanoluciferase protein) mRNA delivered via nebulization at the indicated dosage. FIG. 11A depicts luminescence imaging of ferret lung tissue with Cas13a-NLuc mRNA delivered via nebulization at the indicated dosage FIG. 11B quantifies radiance of ferret lung tissue from luminescence imaging with Cas13a-NLuc mRNA delivered via nebulization at the indicated dosage.

FIG. 12 depicts a chart of average lung radiance vs. administration of aNLuc mRNA (single dose, 0.3 mg/kg) in mouse, hamster and ferret lung tissue with aHCA-NLuc mRNA delivered via nebulization.

FIG. 13 depicts a chart of total flux vs. administration of aNLuc mRNA (single dose, 0.3 mg/kg) in mouse, hamster and ferret lung tissue with aHCA-NLuc mRNA delivered via nebulization.

FIG. 14 depicts a chart of total area vs. administration of aNLuc mRNA (single dose, 0.3 mg/kg) in mouse, hamster and ferret lung tissue with aHCA-NLuc mRNA delivered via nebulization.

FIG. 15 depicts luminescence imaging of cow lung tissue with aNLuc mRNA delivered via nebulization.

FIGS. 16A-16B depict Rhesus macaque lung tissue with aNLuc mRNA delivered via nebulization at 0.3 mg/kg using the indicated polymer. Lungs were imaged at the indicated time post transfection. FIG. 16A depicts luminescence imaging of rhesus macaque lung tissue with aNLuc mRNA delivered via nebulization at 0.3 mg/kg using the indicated polymer at the indicated time post transfection. FIG. 16B depicts a chart quantifying the luminescence imaging of rhesus macaque lung tissue with aNLuc mRNA delivered via polymer 76 by nebulization at 0.3 mg/kg using the indicated polymer at the 4 hours and 24 hours post transfection.

FIG. 17 depicts a chart of fold change total flux vs time post transfection of rhesus macaque lung tissue with aNLuc mRNA delivered via nebulization at 0.3 mg/kg using the indicated polymer.

FIG. 18 depicts a microscopic image of ferret lung with IgG-NLuc or Cas13-NLuc fusion mRNA (white spots) delivered via nebulization at 0.3 mg/kg RNA per animal at 4 hours post transfection.

FIG. 19 depicts a microscopic image of rhesus macaque lung with aNLuc mRNA (white spots) delivered via nebulization at 0.3 mg/kg RNA per animal at 4 hours post transfection.

FIG. 20 depicts a microscopic image of ferret (top) and Rhesus macaque (bottom) lung treated with IgG-NLuc (top left), Cas13a-NLuc (top right), or aNLuc (bottom right) mRNA delivered via nebulization at 0.3 mg/kg RNA per animal at 24 hours post transfection. Lungs were stained with hematoxylin and cosin stain.

FIG. 21 depicts a microscopic image of mouse lung treated with aNLuc (left) mRNA delivered via nebulization at 0.3 mg/kg RNA per animal at 24 hours post transfection. Lungs were stained with hematoxylin and cosin stain.

FIG. 22 depicts a chart of RNA level fold change compared to untreated versus significance in mouse lung treated with aNLuc mRNA delivered via nebulization at 0.3 mg/kg RNA per animal at 4 hours post transfection.

FIGS. 23A and 23B depict size of polyplexes formed by indicated polymers and aNLuc mRNA. FIG. 23A depicts a chart of size of polyplexes formed by indicated polymers and aNLuc mRNA measured by dynamic light scattering before and after nebulization. FIG. 23B depicts microscopic images of size of polyplexes formed by indicated polymers and aNLuc mRNA measured by dynamic cryo-electron microscopy before and after nebulization.

FIG. 24 depicts a chart of surface charge of polyplexes formed by indicated polymers and aNLuc mRNA measured by dynamic light scattering.

FIG. 25 depicts a simulation resultant image of a portion of RNA binding to a portion of polymer 76.

FIGS. 26A-26E depict NMR of lead candidate polymers. FIG. 26A depicts NMR of polymer 38. FIG. 26B depicts NMR of polymer 76. FIG. 26C depicts NMR of polymer 94. FIG. 26D depicts NMR of polymer 116. FIG. 26E depicts NMR of polymer 147.

FIGS. 27A-27B depict NMR of polymers. FIG. 27A depicts ¹H NMR of the indicated polymers in the region characteristic of N-formyl (RR′N-CHO) substitution. FIG. 27B depicts ¹³C NMR of hDD90-118 prepared in the presence of 10% ¹³C-enriched N,N-dimethylformamide, under otherwise identical conditions to those reported in this document. All the ¹³C-enriched peaks marked with asterisks are assigned to N-formyl (N—CHO) groups.

FIG. 28 depicts the mass ration testing of polymer 76. aNLuc mRNA was formulated with P76 at the indicated mass ratio. Polyplexes were nebulized at a dose of 25 μg/mouse and lungs were evaluated at 24 hours for aNLuc protein expression.

FIG. 29 depicts flux quantified in mouse lungs from animals treated with mRNA made fresh or lyophilized after 24 hours.

FIG. 30 depicts a microscopic image of hamster lungs with beta-galactosidase immunofluorescence staining (white) expression of beta-gal mRNA delivered via nebulization.

FIG. 31 depicts a microscopic image of mice lungs with beta-galactosidase immunofluorescence staining (white) expression of beta-gal mRNA delivered via nebulization.

FIGS. 32A-32B depict efficacy of nebulizers in delivering mRNA to the lung and trachea of bovine at a 0.07 mg/kg dose. FIG. 32A depicts representative images of polymer 76 delivering mRNA via different nebulization techniques in bovine lung and trachea. FIG. 32B depicts quantification of polymer 76 delivering mRNA via different nebulization techniques in bovine lung and trachea.

FIGS. 33A-33B depict polymer 76 delivery of mRNA in swine lungs at a 0.7 mg/kg dose. FIG. 33A depicts representative images of polymer 76 delivering mRNA via jet nebulization in swine lung. FIG. 33B depicts quantification of polymer 76 delivering mRNA via jet nebulization in swine lung.

FIGS. 34A-C depict a toxicity study in mice treated with aNLuc mRNA delivered via polymer 76 at a single 1.25 mg/kg dose. FIG. 34A depicts mouse body weights as a percentage of starting weight for mice treated with aNLuc and acetate. FIG. 34B depicts a schematic of enzyme-linked immunoassay (ELISA) to detect mouse anti-P76 polyplex antibody responses at days 1, 7, 14 and 21. Horseradish peroxidase (HRP); 3,3′, 5,5′-Tetramethylbenzidine (TMB). FIG. 34C depicts mouse anti-P76 polyplex antibodies detected via ELISA.

FIGS. 35A-35D depict blood chemistry metrics in mice treated with aNLuc mRNA delivered via polymer 76 at a single 1.25 mg/kg dose at a range of timepoints. FIG. 35A depicts blood chemistry metrics for ALT and AST in mice with a single 1.25 mg/kg dose at the listed timepoints (D: day). FIG. 35B depicts blood chemistry metrics for calcium and urea-nitrogen in mice with a single 1.25 mg/kg dose at the listed timepoints (D: day). FIG. 35C depicts blood chemistry metrics for total protein and phosphorus in mice with a single 1.25 mg/kg dose at the listed timepoints (D: day). FIG. 35D depicts blood chemistry metrics for creatine and triglycerides in mice with a single 1.25 mg/kg dose at the listed timepoints (D: day).

FIG. 36 depicts differential gene expression of 561 inflammatory genes in mice treated with aNLuc mRNA delivered via polymer 76 at a single 1.25 mg/kg dose at a range of timepoints.

FIG. 37 depicts mouse lungs stained with hematoxylin and cosin from mice treated with aNLuc mRNA delivered via polymer 76 at a single 1.25 mg/kg dose at a range of timepoints.

FIGS. 38A-38B depict flux in hamster lungs when nebulized with formulations of polymer 76. FIG. 38A depicts the quantification of hamster lung luminescence at 24 h post transfection of 1.25 mg kg−1 of Cas13a-NLuc mRNA. FIG. 38B depicts fold change total flux of hamster lungs 24 h after delivery of the indicated total dose of Cas 13-NLuc mRNA and crRNA.

FIGS. 39A-39F depict treatment of mice with nebulized polymer 76 formation for the treatment of SARS-COV-2 in a hamster model. FIG. 39A depicts a schematic of the SARC-CoV-2 treatment regimen. FIG. 39B depicts a chart showing percent normalized hamster weight over time. FIG. 39C depicts a chart showing percent hamster weight at day 5 post infection. FIG. 39D depicts a chart showing percent normalized hamster weight over time. FIG. 39E depicts a chart showing percent hamster weight at day 5 post infection. FIG. 39F depicts a chart showing percent knockdown of SARS-COV-2 RNA in the lung at day 5 post infection

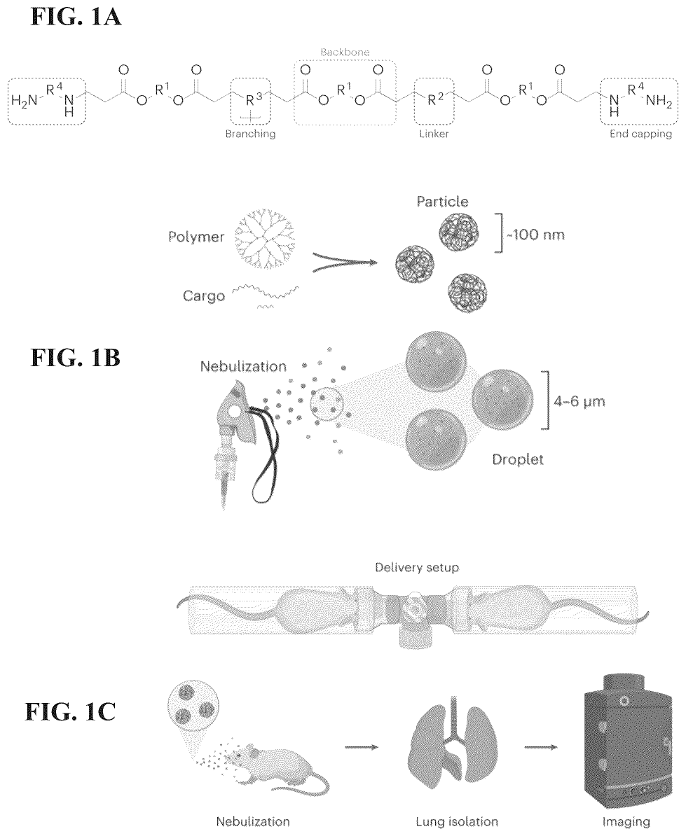

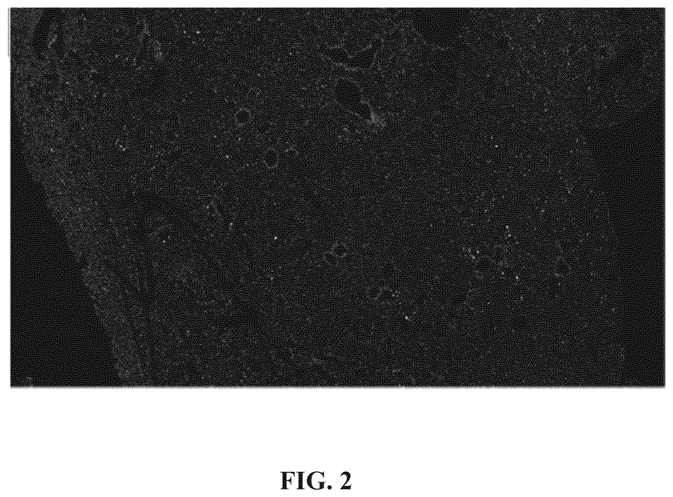

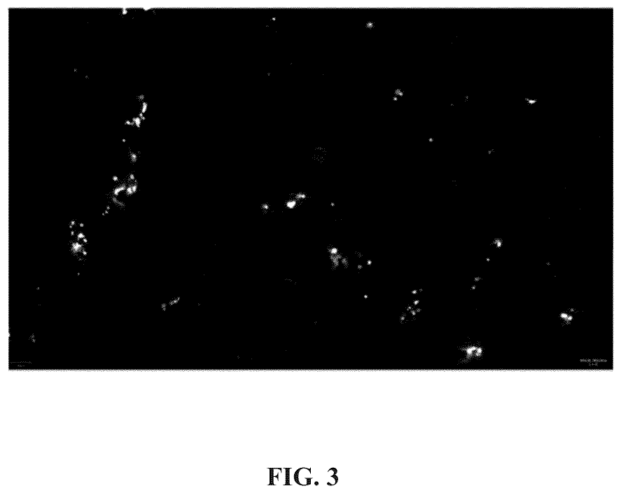

### DETAILED DESCRIPTION

The presently disclosed inventive subject matter may be understood more readily by reference to the following detailed description taken in connection with the accompanying figures and examples, which form a part of this disclosure. It is to be understood that these inventions are not limited to the specific components, methods, or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

The present disclosure relates to inventive BAE/BTE-containing polymers, which useful for the non-viral delivery of agents (e.g., nucleic acids) to cells. In certain embodiments, polymers of the invention are useful for the delivery of mRNA to both lung endothelium and epithelium via nebulization, dry powder inhalation, or systemic administration, and are therefore clinically relevant to the treatment of infections and disorders of the lung epithelium, including respiratory virus infections, bacterial infections, enzyme deficiencies, and cystic fibrosis.

### Polymers of Formula (I)

In one aspect, provided herein is a BAE/BTE-containing polymer of Formula (I) comprising a backbone of Formula (A), an optional linking group of Formula (B), an optional branching group of Formula (C), and an optional end-capping group of Formula (D).

In one aspect, provided herein is a BAE/BTE-containing polymer of Formula (I) comprising a backbone of Formula (A), a linking group of Formula (B), an optional branching group of Formula (C), and an optional end-capping group of Formula (D).

In one aspect, provided herein is a BAE/BTE-containing polymer of Formula (I) comprising a backbone of Formula (A), a linking group of Formula (B), a branching group of Formula (C), and an optional end-capping group of Formula (D).

In one aspect, provided herein is a BAE/BTE-containing polymer of Formula (I) comprising a backbone of Formula (A), a linking group of Formula (B), a branching group of Formula (C), and an end-capping group of Formula (D).

In as aspect, the backbone of Formula (A) comprises an electrophilic structure selected from:

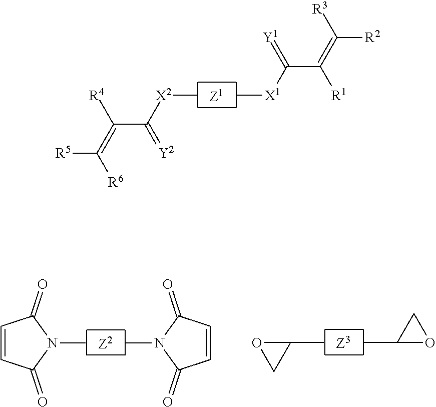

wherein:
Z¹, Z² and Z³ are independently selected from alkyl, aryl, heteroaryl, and a polycyclic structure, and each of Z¹, Z² and Z³ is independently optionally substituted with at least one group selected from hydroxyl, carboxylic acid, carboxylic ester, amide, thioester, urea, imide, alkene, alkyne, ether, thioether, tertiary amine, phosphonate, sulfoxide, sulfone, imine, oxime, hydrazide, borane and borate;
X¹ and X² are independently O or N;
Y¹ and Y² are independently O, NR or S;
R is H, alkyl, aryl, heteroaryl or a polycyclic structure, and is optionally substituted with hydroxyl, carboxylic acid, carboxylic ester, amide, thioester, urea, imide, alkene, alkyne, ether, thioether, tertiary amine, phosphonate, sulfoxide, sulfone, imine, oxime, hydrazide, borane, borate, or any combination thereof; and
R¹, R², R³, R⁴, R⁵, and R⁶ are independently H, C₁₋₆alkyl, or aryl.

In some embodiments, the backbone of Formula (A) is

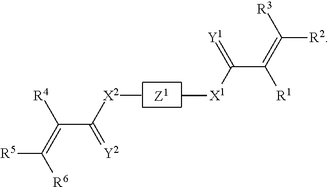

In some embodiments, Z¹ is alkyl. In some embodiments, Z¹ is aryl. In some embodiments, Z¹ is heteroaryl. In some embodiments, Z¹ is a polycyclic structure. In some embodiments, Z¹ is a bicyclic structure.

In some embodiments, at least one of X¹ and X² is O. In some embodiments, at least one of X¹ and X² is N. In some embodiments, both of X¹ and X² are O. In some embodiments, both of X¹ and X² are N. In some embodiments, X¹ is O and X² is N. In some embodiments, X¹ is N and X² is O.

In some embodiments, at least one of Y¹ and Y² is O. In some embodiments, at least one of Y¹ and Y² is NR. In some embodiments, at least one of Y¹ and Y² is S. In some embodiments, both of Y¹ and Y² are O. In some embodiments, both of Y¹and Y² are NR. In some embodiments, both of Y¹ and Y² are S.

In some embodiments, Y¹ is O and Y² is NR. In some embodiments, Y¹ is O and Y² is S. In some embodiments, Y¹ is NR and Y² is O. In some embodiments, Y¹ is NR and Y² is S. In some embodiments, Y¹ is S and Y² is NR. In some embodiments, Y¹ is S and Y² is O.

In some embodiments, R is H, alkyl, aryl, heteroaryl, or a polycyclic structure, and is optionally substituted with hydroxyl, carboxylic acid, carboxylic ester, amide, thioester, urea, imide, alkene, alkyne, ether, thioether, tertiary amine, phosphonate, sulfoxide, sulfone, imine, oxime, hydrazide, borane, borate or any combination thereof.

In some embodiments, R is H. In some embodiments, R is alkyl. In some embodiments, R is aryl. In some embodiments, R is heteroaryl. In some embodiments, R is a polycyclic structure. In some embodiments, R is methyl. In some embodiments, R is ethyl. In some embodiments, R is propyl. In some embodiments, R is isopropyl. In some embodiments, R is phenyl. In some embodiments, R is pyridyl.

In some embodiments, at least one of R¹, R², R³, R⁴, R⁵, R⁶ is H. In some embodiments, at least two of R¹, R², R³, R⁴, R⁵, R⁶ are H. In some embodiments, at least three of R¹, R², R³, R⁴, R⁵, R⁶ are H. In some embodiments, at least four of R¹, R², R³, R⁴, R⁵, R⁶ are H. In some embodiments, at least five of R¹, R², R³, R⁴, R⁵, R⁶ are H. In some embodiments, at least six of R¹, R², R³, R⁴, R⁵, R⁶ are H. In some embodiments, cach of R¹, R², R³, R⁴, R⁵, R⁶ is H.

In some embodiments, at least one of R¹, R², R³, R⁴, R⁵, R⁶ is C₁₋₆alkyl. In some embodiments, at least two of R¹, R², R³, R⁴, R⁵, R⁶ are C₁₋₆alkyl. In some embodiments, at least three of R¹, R², R³, R⁴, R⁵, R⁶ are C₁₋₆alkyl. In some embodiments, at least four of R¹, R², R³, R⁴, R⁵, R⁶ are C16alkyl. In some embodiments, at least five of R¹, R², R³, R⁴, R⁵, R⁶ are C₁₋₆alkyl. In some embodiments, at least six of R¹, R², R³, R⁴, R⁵, R⁶ are C₁₋₆alkyl. In some embodiments, cach of R¹, R², R³, R⁴, R⁵, R⁶ is C₁₋₆alkyl.

In some embodiments, the C₁₋₆alkyl is a methyl group. In some embodiments, the C₁₋₆alkyl is an ethyl group. In some embodiments, the C₁₋₆alkyl is an n-propyl group. In some embodiments, the C₁₋₆alkyl is an isopropyl group. In some embodiments, the C₁₋₆alkyl is an n-butyl group. In some embodiments, the C₁₋₆alkyl is an isobutyl group. In some embodiments, the C₁₋₆alkyl is an n-pentyl group. In some embodiments, the C₁₋₆alkyl is an isopentyl group. In some embodiments, the C₁₋₆alkyl is a neopentyl group. In some embodiments, the C₁₋₆alkyl is an n-hexyl group.

In some embodiments, at least one of R¹, R², R³, R⁴, R⁵, R⁶ is aryl. In some embodiments, at least two of R¹, R², R³, R⁴, R⁵, R⁶ are aryl. In some embodiments, at least three of R¹, R², R³, R⁴, R⁵, R⁶ are aryl. In some embodiments, at least four of R¹, R², R³, R⁴, R⁵, R⁶ are aryl. In some embodiments, at least five of R¹, R², R³, R⁴, R⁵, R⁶ are aryl. In some embodiments, at least six of R¹, R², R³, R⁴, R⁵, R⁶ are aryl. In some embodiments, each of R¹, R², R³, R⁴, R⁵, R⁶ is aryl.

In some embodiments, the aryl is a phenyl group.

In some embodiments, the backbone of Formula (A) is

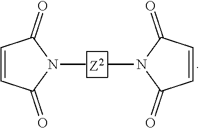

In some embodiments, Z² is alkyl. In some embodiments, Z² is aryl. In some embodiments, Z² is heteroaryl. In some embodiments, Z² is a polycyclic structure. In some embodiments, Z² is a bicyclic structure.

In some embodiments, the backbone of Formula (A) is

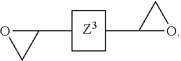 [SMILES: C1CC(C2CO2)CCC1C1CO1]

In some embodiments, Z³ is alkyl. In some embodiments, Z³ is aryl. In some embodiments, Z³ is heteroaryl. In some embodiments, Z³ is a polycyclic structure. In some embodiments, Z³ is a bicyclic structure.

In some embodiments, the backbone of Formula (A) is selected from:

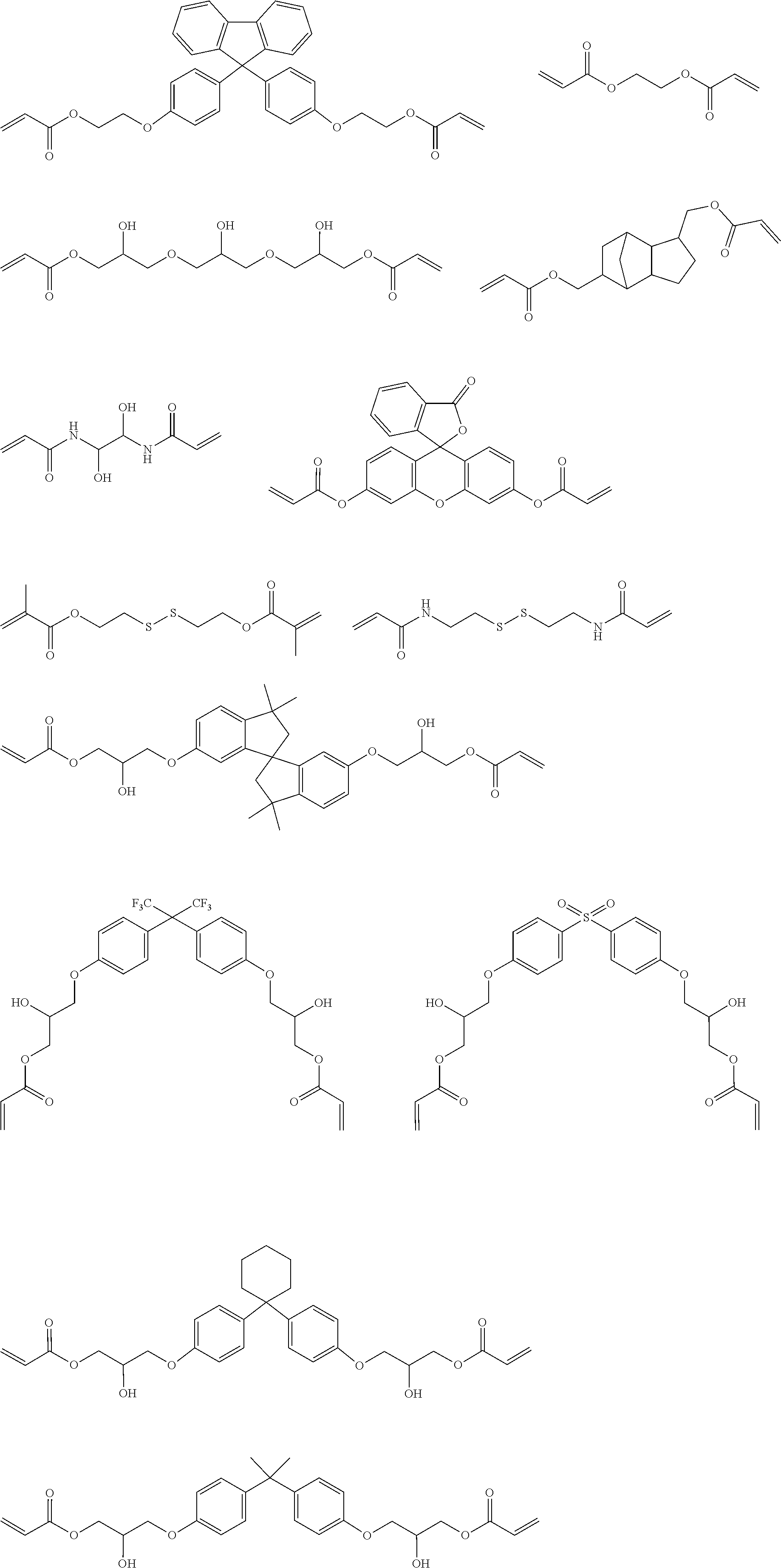

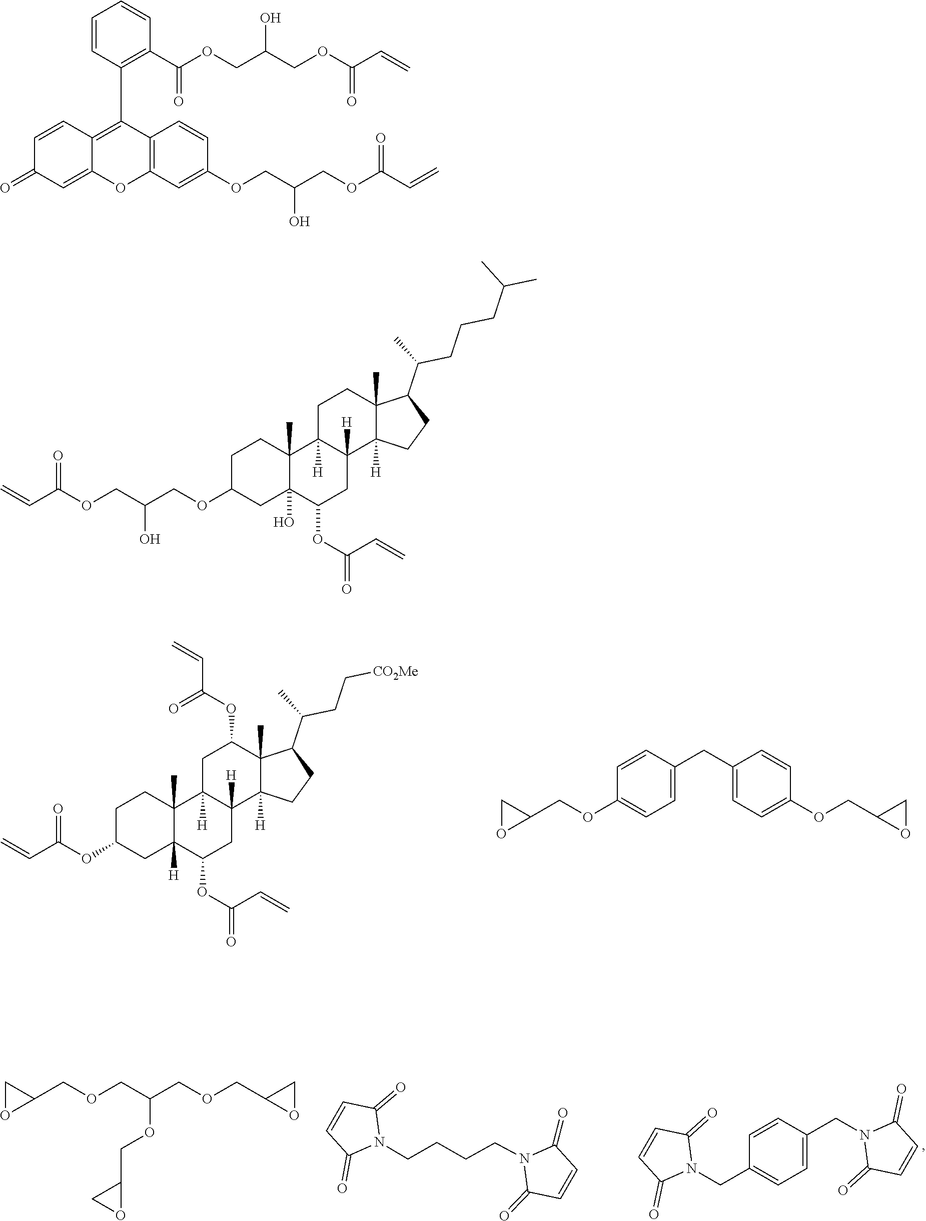

or any combination thereof.

In some aspects, the linking group of Formula (B) is selected from HS—R⁷—SH and R⁷—NH₂; wherein R⁷ is alkyl, aryl, carbocyclic, heteroaryl and heterocyclic.

In some embodiments, the linking group of Formula (B) is HS—R⁷—SH. In some embodiments, the linking group of Formula (B) is R⁷—NH₂.

In some embodiments, R⁷ is alkyl. In some embodiments, R⁷ is aryl. In some embodiments, R⁷ is carbocyclic. In some embodiments, R⁷ is heteroaryl. In some embodiments, R⁷ is heterocyclic.

In some embodiments, the linking group of Formula (B) is

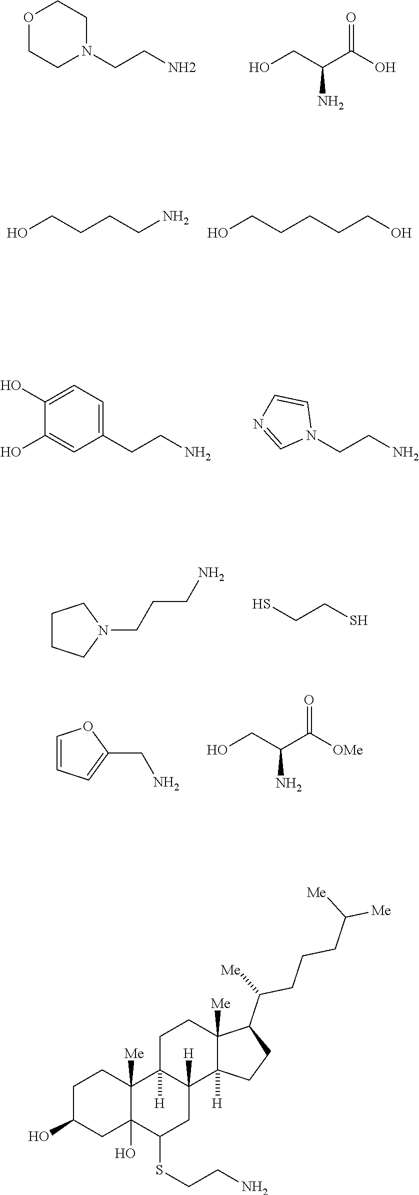

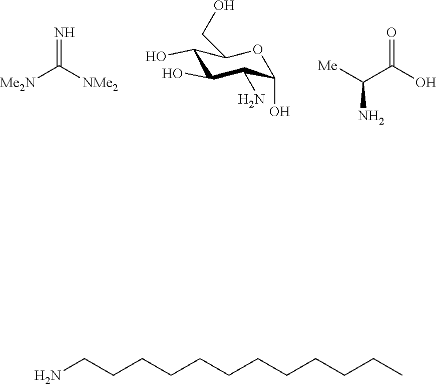

or any combination thereof.

In some aspects, the branching group of Formula (C) is selected from HS—R⁸—NH₂, H₂N—R⁸—NH₂ and R⁸—HN—R⁸—NH₂; wherein R⁸ is alkyl, aryl, carbocyclic, heteroaryl and heterocyclic.

In some embodiments, the branching group of Formula (C) is HS—R⁸—NH₂. In some embodiments, the branching group of Formula (C) is H₂N—R⁸—NH₂. In some embodiments, the branching group of Formula (C) is R⁸—HN—R⁸—NH₂.

In some embodiments, R⁸ is alkyl. In some embodiments, R⁸ is aryl. In some embodiments, R⁸ is carbocyclic. In some embodiments, R⁸ is heteroaryl. In some embodiments, R⁸ is heterocyclic.

In some embodiments, the branching group of Formula (C) is

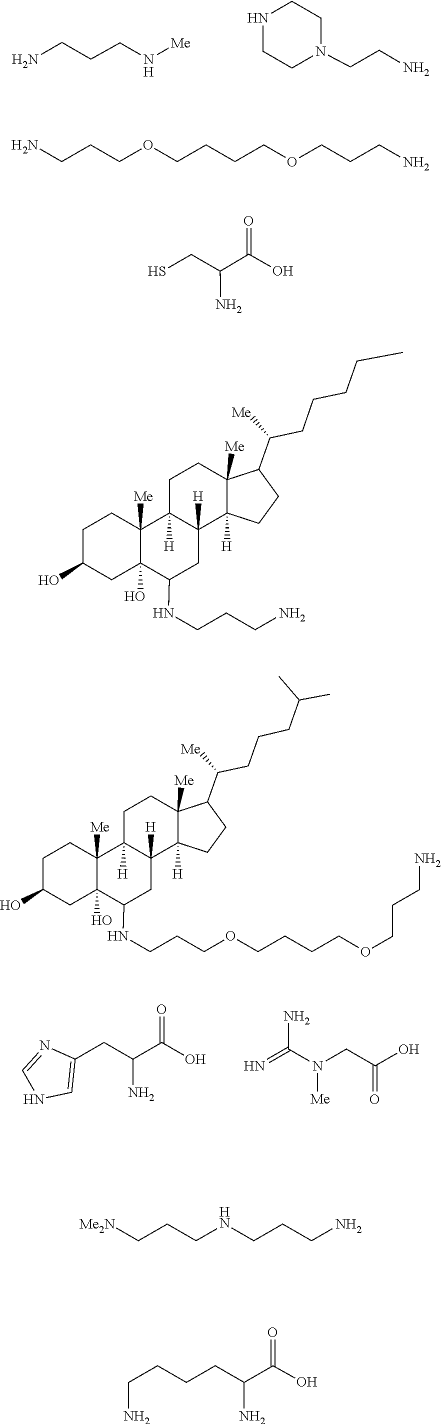

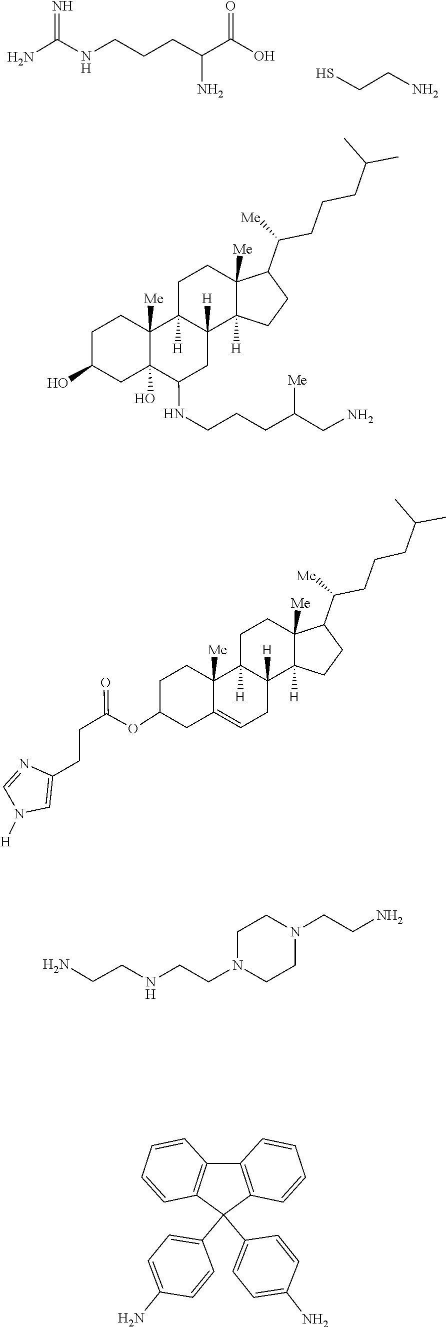

or any combination thereof.

In as aspect, the end-capping group of Formula (D) is selected from HS—R⁹—NH₂, H₂N—R⁹—NH₂ and R⁹—HN—R⁹—NH₂; wherein R⁹ is alkyl, aryl, carbocyclic, heteroaryl and heterocyclic.

In some embodiments, the end-capping group of Formula (D) is HS—R⁹—NH₂. In some embodiments, the end-capping group of Formula (D) is H₂N—R⁹—NH₂. In some embodiments, the end-capping group of Formula (D) is R⁹—HN—R⁹—NH₂.

In some embodiments, R⁹ is alkyl. In some embodiments, R⁹ is aryl. In some embodiments, R⁹ is carbocyclic. In some embodiments, R⁹ is heteroaryl. In some embodiments, R⁹ is heterocyclic.

In some embodiments, the end-capping group of Formula (D) is

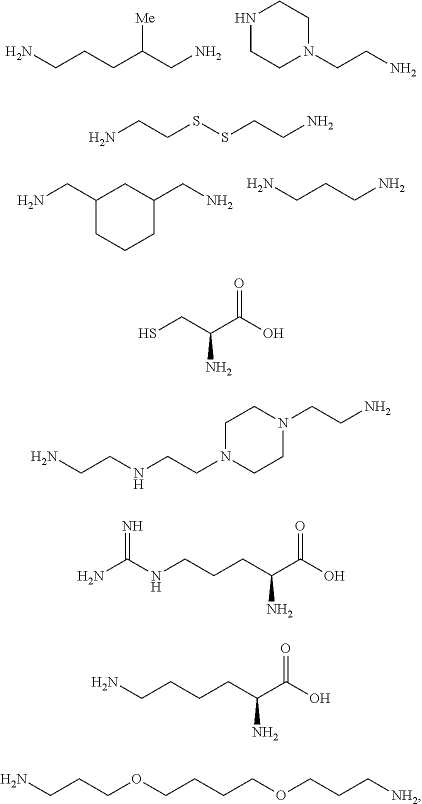

or any combination thereof.

In certain embodiments, each Formula (A) has two points of attachment to the optional groups of Formulae (B), (C), and (D);
each Formula (B) has at least one point of attachment to radicals of Formula (A);
each Formula (C) has at least two points of attachment to radicals of Formula (A);
each Formula (D) has at least one point of attachment to a radical of Formula (A); and
wherein Formulae (B), (C), and (D), if present, may be the same or different from one another.

The polymer of Formula (I) comprises radicals of Formulae (A), (B), (C), and (D) in various orders, arrangements, and molar percentages. By varying the molar percentages of one or more of the component radicals, it is possible to control the degree of branching, relative abundance of primary amines (e.g., primary amine termini), and thereby to control properties such as aqueous solubility and transfection efficiency.

In certain embodiments the linker has at least 1 thiol. In certain embodiments, the linker comprises a dithiol in a molar ratio of at least 0.05. In certain embodiments the linker comprises ethanedithiol in a molar ratio of 0.1 By varying the molar percentages of one or more of the component thiols, it is possible to control the binding and release of mRNA from the polyplex, transfection efficiency, particle size, and stability of the polyplex.

In certain embodiments, the polymer represents any combination of radicals and repeated radicals listed in A, B represents any combination of radicals and repeat radicals listed in B, C represents any combination of radicals and repeated radicals listed in C, and D represents any combination of radicals and repeated radicals listed in D.

In certain embodiments, the polymer of Formula (I) comprises radicals of Formulac (A) and (B) in a molar ratio of about 1:0 to about 1:1. In certain embodiments, the polymer of Formula (I) comprises radicals of Formulac (A) and (C) in a molar ratio of about 1:0 to about 1:0.8. In certain embodiments, the polymer of Formula (I) comprises radicals of Formulac (A) and (D) in a molar ratio of about 1:0.1 to about 1:1.5.

### Branching

As used herein, the term “branched” refers to polymers containing branches that are composed of the same units that make up the linear portion of the main chain. As used herein, the term “hyperbranched” refers to polymers containing branches that are composed of the units that make up the linear portion of the main chain as well as further branch points (e.g., radicals of Formula (C), also referred to as “dendritic units”). Hyperbranched dendritic polymers contain randomly distributed dendritic units and offer a large chemical space for investigation as they can be synthesized with a wide range of monomers using one-pot reaction conditions. Linear segments can be combined with hyperbranched segments to alter the degree of branching (DB), thereby altering properties such as solubility, viscosity, and efficacy as a transfection reagent.

The terms “Degree of branching” and “DB” can be defined as the ratio of dendritic units (radicals of Formula (C)) to linear units (radicals of Formulae (A), (B), and (D)). DB can be calculated using the equation: DB=(D+T)/(D+T+L), where D is number of dendritic units, T is the number of terminal units (radical of Formula (D)) and L is number of linear units (radicals of Formulac (A) and (B)). (See also, Hawker & Fretchet 1991 J. Am. Chem. Soc. 113 (12)). DB can be controlled as a function of the stoichiometry of Formula (B) to Formula (C). For example, higher DB is obtained by using a molar excess of the monomer corresponding to Formula (B), relative to the monomer corresponding to Formula (C). Linear polymers (DB=0) are obtained by omitting the monomer corresponding to Formula (C).

DB also correlates directly with an increase in terminal primary amine groups. Increased density of primary amines in the BAE/BTE-containing polymers may influence polymer efficacy as a transfection reagent at various stages during the formulation and transfection process, for example, during nanoparticle formulation, discussed in more detail below, when the cationic polymer protects nucleic acid cargo through electrostatic condensation to prevent degradation by nucleases.

In certain embodiments, the polymers of the invention are linear. In certain embodiments, the polymers of the invention are branched or hyperbranched. In certain embodiments, the polymers of the invention are about 80, 81, 82, 83, 84, 85, 86, 87, 88, 89, 90, 91, 92, 93, 94, 95, 96, 97, 98, 99, or 100% branched.

DB influences properties such as intrinsic viscosity, solubility, and transfection efficacy. In certain embodiments, the polymer of Formula (I) has a degree of branching (DB) in the range of 0.0-1.0. In certain embodiments, the polymer of Formula (I) has a degree of branching (DB) in the range of 0.0-0.5. In certain particular embodiments, the DB of a polymer of Formula (I) is 0.0, about 0.1, about 0.2, about 0.3, about 0.4, about 0.5, about 0.6, or about 0.7.

In certain embodiments, the polymers of the invention are biodegradable or biocompatible. As used herein, “biodegradable” polymers are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably do not induce inflammation or other adverse effects in vivo. In certain embodiments, the chemical reactions relied upon to break down the biodegradable polymers are uncatalyzed. Biodegradability is a particular advantage of these BAE/BTE-containing delivery vectors, particularly for repeat administration where non-degradable vectors like PEI may accumulate or be difficult for the body to metabolize. The term “biocompatible,” as used herein is intended to describe compounds that are not toxic to cells. Polymers are “biocompatible” if their addition to cells in vitro results in less than or equal to 20% cell death, and their administration in vivo does not induce inflammation or other such adverse effects.

### Formula (A)

Formula (A) is a backbone having two points of attachment to radicals selected from Formulae (B), (C), and (D).

In certain embodiments, the polymers of the invention (e.g., a polymer of Formula (I)) comprise 1 eq of Formula (A). In certain embodiments, the polymers of the invention comprise 1-1.5 eq of Formula (A).

In certain embodiments, all radicals of Formula (A) are the same. In other embodiments, there are two or more (e.g., 2, 3, or 4) different radicals of Formula (A).

In certain embodiments, Formula (A) comprises:

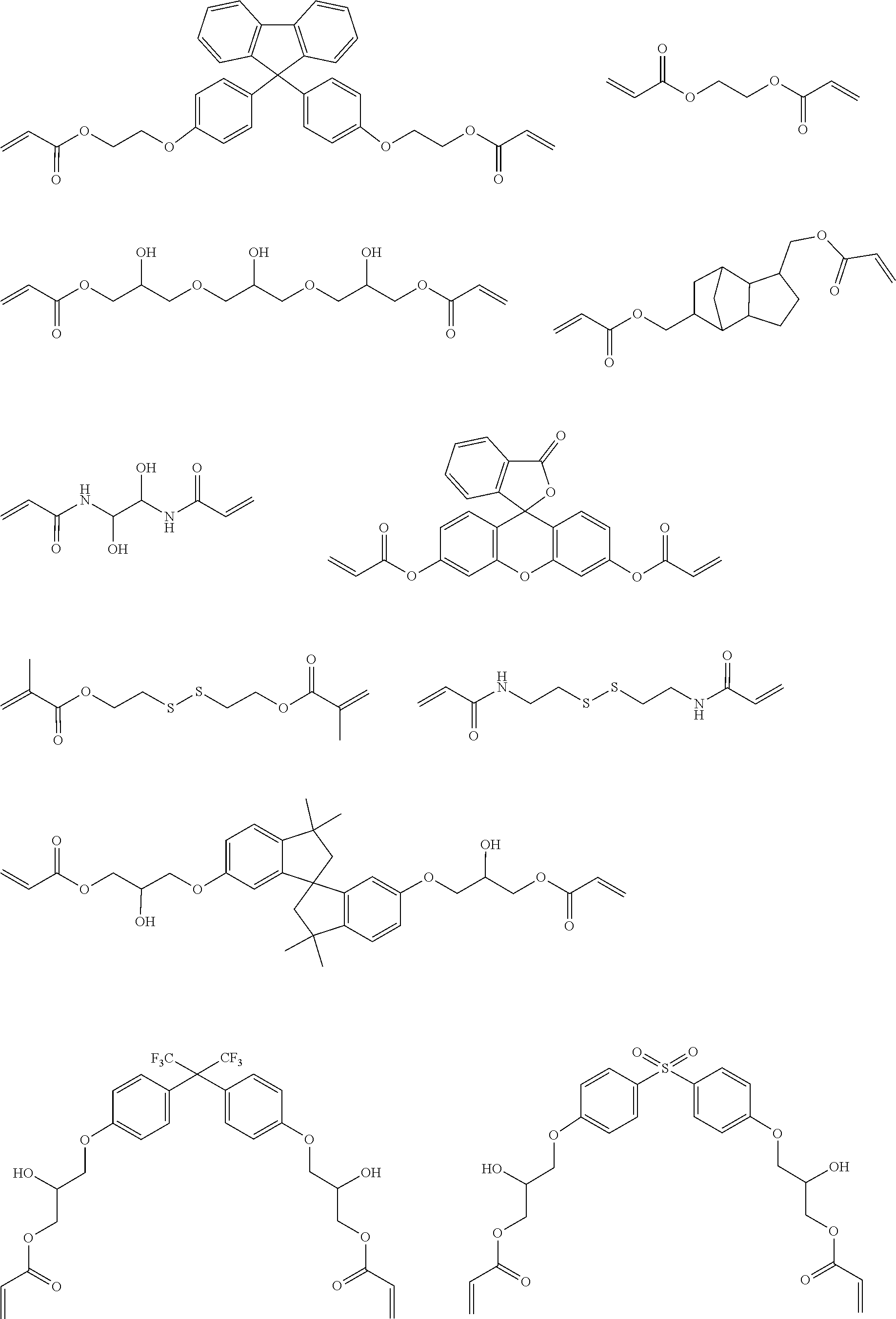

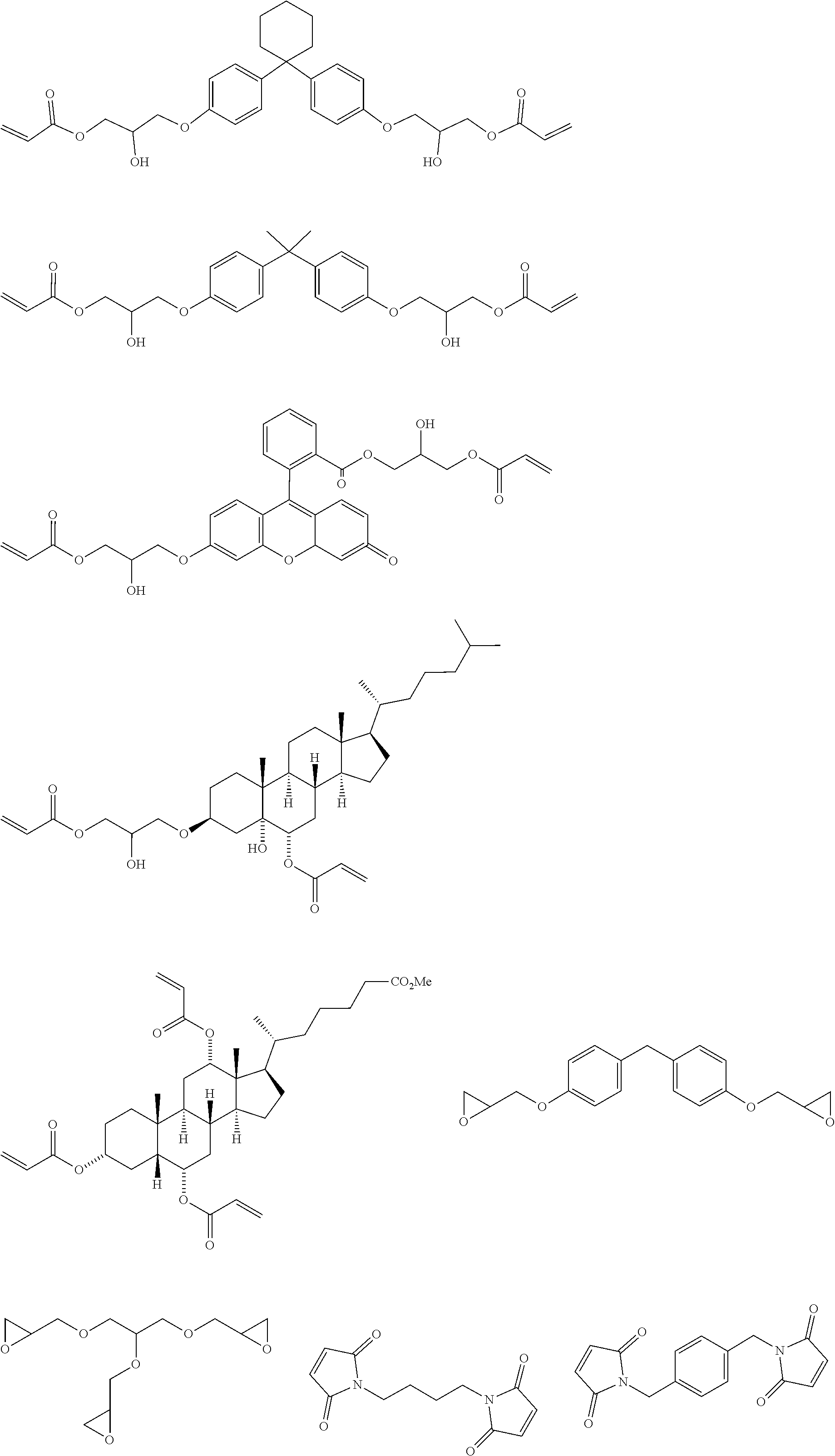

or any combination thereof.

In certain embodiments, Formula (A) comprises

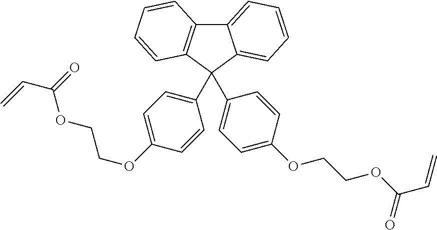 [SMILES: C=CC(=O)OCCOc1ccc(C2(c3ccc(OCCOC(=O)C=C)cc3)c3ccccc3-c3ccccc32)cc1]

In certain embodiments, Formula (A) comprises

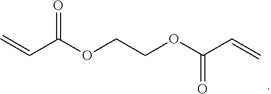

In certain embodiments, Formula (A) comprises

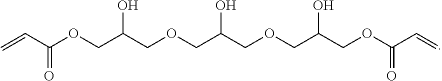

In certain embodiments, Formula (A) comprises

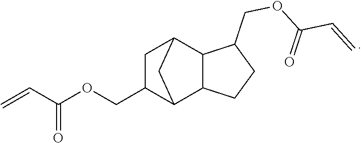 [SMILES: C=CC(=O)OCC1CC2CC1C1CCC(COC(=O)C=C)C21]

In certain embodiments, Formula (A) comprises

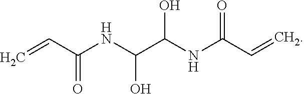

In certain embodiments, Formula (A) comprises

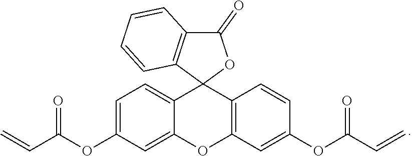 [SMILES: C=CC(=O)Oc1ccc2c(c1)Oc1cc(OC(=O)C=C)ccc1C21OC(=O)c2ccccc21]

In certain embodiments, Formula (A) comprises

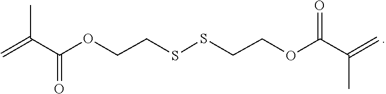 [SMILES: C=C(C)C(=O)OCCSSCCOC(=O)C(=C)C]

In certain embodiments, Formula (A) comprises

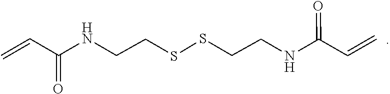 [SMILES: C=CC(=O)NCCSSCCNC(=O)C=C]

In certain embodiments, Formula (A) comprises

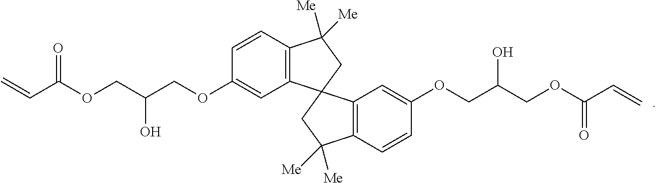 [SMILES: C=CC(=O)OCC(O)COc1ccc2c(c1)C1(CC2(C)C)CC(C)(C)c2ccc(OCC(O)COC(=O)C=C)cc21]

In certain embodiments, Formula (A) comprises

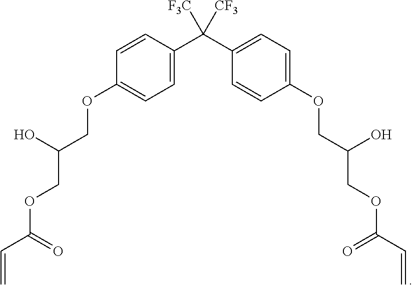 [SMILES: C=CC(=O)OCC(O)COc1ccc(C(c2ccc(OCC(O)COC(=O)C=C)cc2)(C(F)(F)F)C(F)(F)F)cc1]

In certain embodiments, Formula (A) comprises

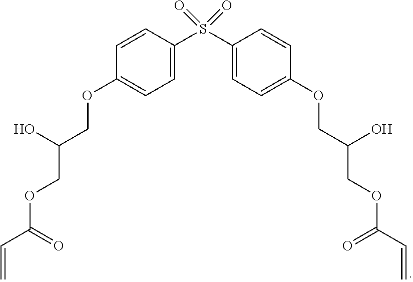 [SMILES: C=CC(=O)OCC(O)COc1ccc(S(=O)(=O)c2ccc(OCC(O)COC(=O)C=C)cc2)cc1]

In certain embodiments, Formula (A) comprises

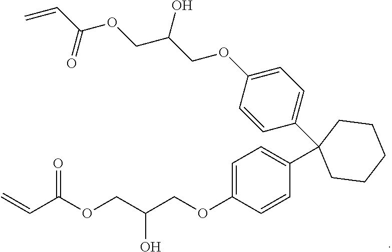 [SMILES: C=CC(=O)OCC(O)COc1ccc(C2(c3ccc(OCC(O)COC(=O)C=C)cc3)CCCCC2)cc1]

In certain embodiments, Formula (A) comprises

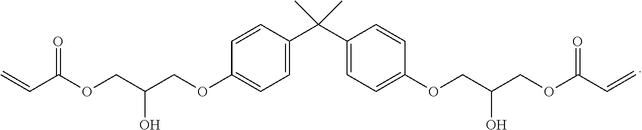 [SMILES: C=CC(=O)OCC(O)COc1ccc(C(C)(C)c2ccc(OCC(O)COC(=O)C=C)cc2)cc1]

In certain embodiments, Formula (A) comprises

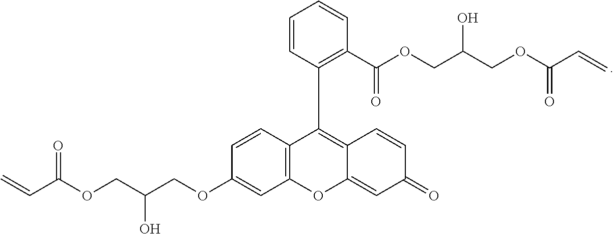 [SMILES: C=CC(=O)OCC(O)COC(=O)c1ccccc1-c1c2ccc(=O)cc-2oc2cc(OCC(O)COC(=O)C=C)ccc12]

In certain embodiments, Formula (A) comprises

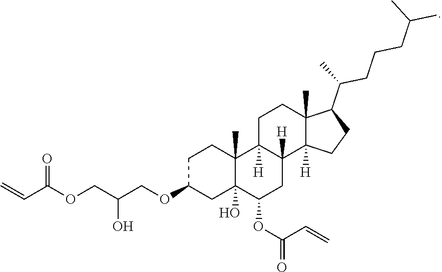

In certain embodiments, Formula (A) comprises

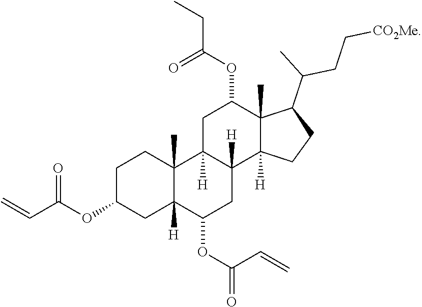

In certain embodiments, Formula (A) comprises

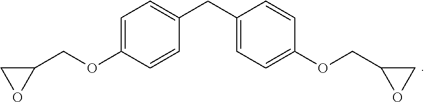 [SMILES: c1cc(OCC2CO2)ccc1Cc1ccc(OCC2CO2)cc1]

In certain embodiments, Formula (A) comprises

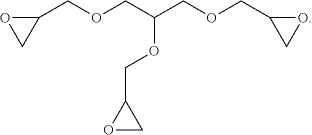 [SMILES: C(OCC1CO1)C(COCC1CO1)OCC1CO1]

In certain embodiments, Formula (A) comprises

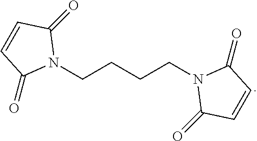 [SMILES: O=C1C=CC(=O)N1CCCCN1C(=O)C=CC1=O]

In certain embodiments, Formula (A) comprises

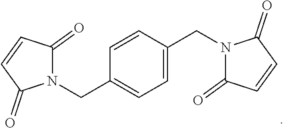 [SMILES: O=C1C=CC(=O)N1Cc1ccc(CN2C(=O)C=CC2=O)cc1]

In certain embodiments, Formula (A) comprises a combination of any one of the above compounds of Formula (A). In certain embodiments, Formula (A) comprises a combination of two of the above compounds of Formula (A). In certain embodiments, Formula (A) comprises a combination of three of the above compounds of Formula (A). In certain embodiments Formula (A) comprises a combination of more than three of the above compounds of Formula (A). In any of the embodiments described, the components of Formula (A) may be present in any relative ratio.

In certain embodiments, the two compounds of Formula (A) in the combination are

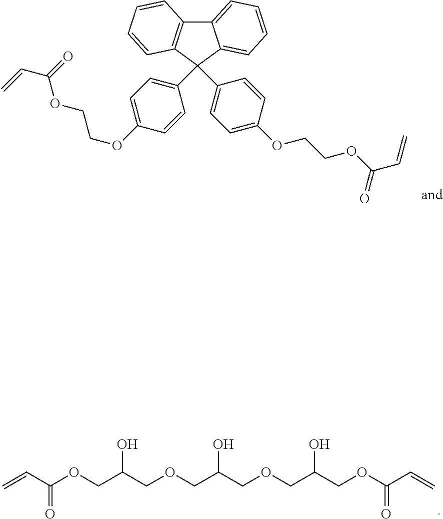

In certain embodiments, the two compounds of Formula (A) in the combination are

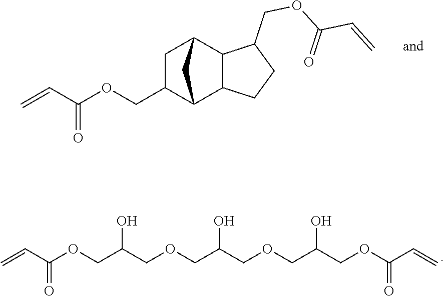

In certain embodiments, the two compounds of Formula (A) in the combination are

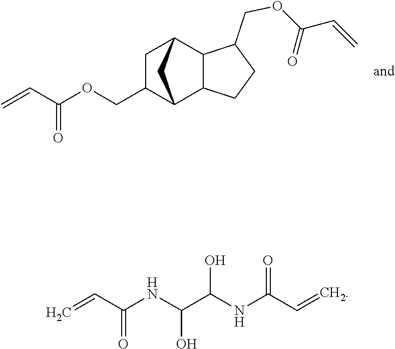

In certain embodiments, the two compounds of Formula (A) in the combination are

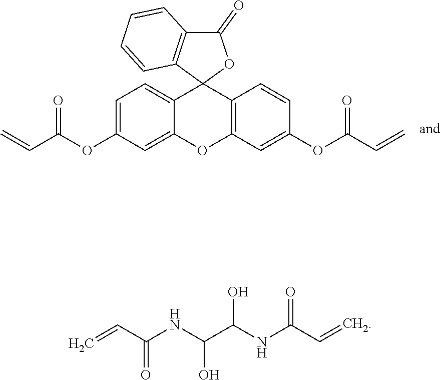

In certain embodiments, the two compounds of Formula (A) in the combination are

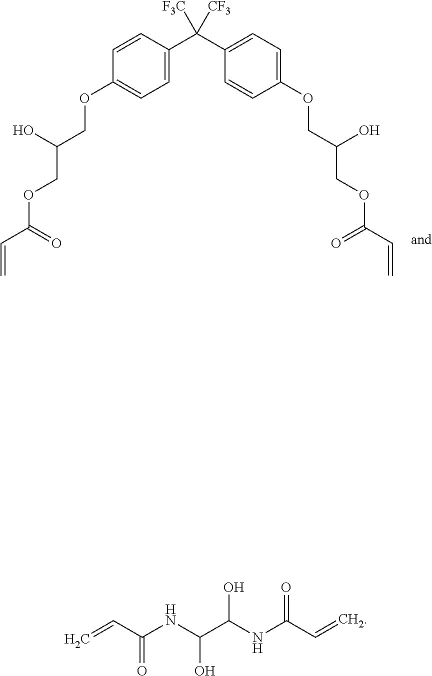

In certain embodiments, the two compounds of Formula (A) in the combination are

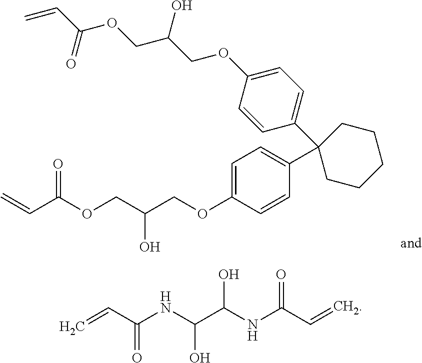 [SMILES: C=CC(=O)OCC(O)COc1ccc(C2(c3ccc(OCC(O)COC(=O)C=C)cc3)CCCCC2)cc1 ; C=CC(=O)NC(O)C(O)NC(=O)C=C]

In certain embodiments, the two compounds of Formula (A) in the combination are

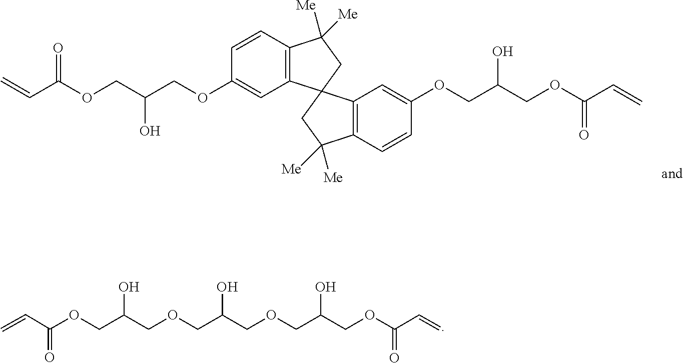

In certain embodiments, the two compounds of Formula (A) in the combination are

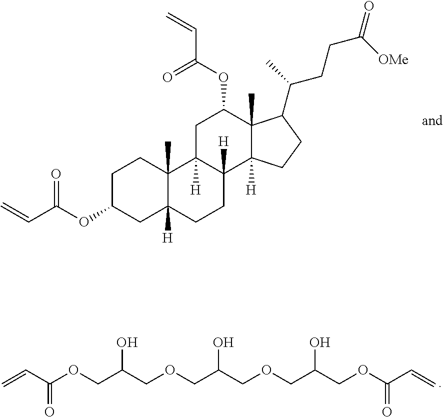

In certain embodiments, the two compounds of Formula (A) in the combination are

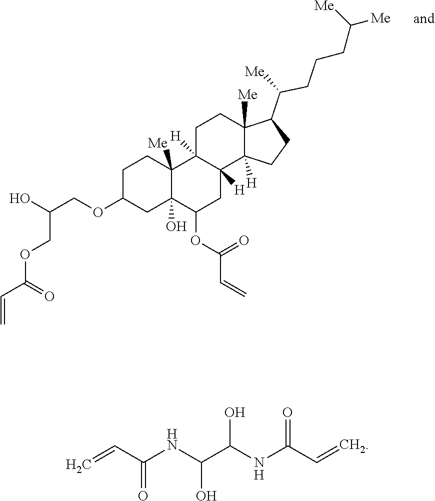 [SMILES: C=CC(=O)OCC(O)COC1CC[C@]2(C)[C@H]3CC[C@]4(C)[C@@H]([C@H](C)CCCC(C)C)CC[C@H]4[C@@H]3CC(OC(=O)C=C)[C@@]2(O)C1 ; C=CC(=O)NC(O)C(O)NC(=O)C=C]

In certain embodiments, the two compounds of Formula (A) in the combination are

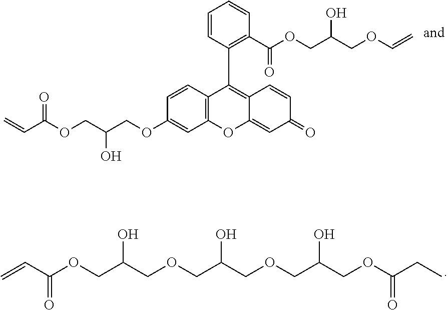

### Formula (B)

Formula (B) is an optional linking group having two points of attachment to radicals of Formula (A). Typically, Formula (B) joins to two different Formula (A) moieties.

In certain embodiments, Formula (B) is absent.

In certain embodiments, the polymers of the invention (e.g., a polymer of Formula (I) or (II)) comprise about 0 to 0.9 of Formula (B). In certain embodiments, the polymers of the invention comprise about 0.1 to 0.4 of Formula (B).

In certain embodiments, all radicals of Formula (B) are the same. In other embodiments, there are two or more (e.g., 2, 3 or 4) different radicals of Formula (B).

In certain embodiments, Formula (B) comprises:

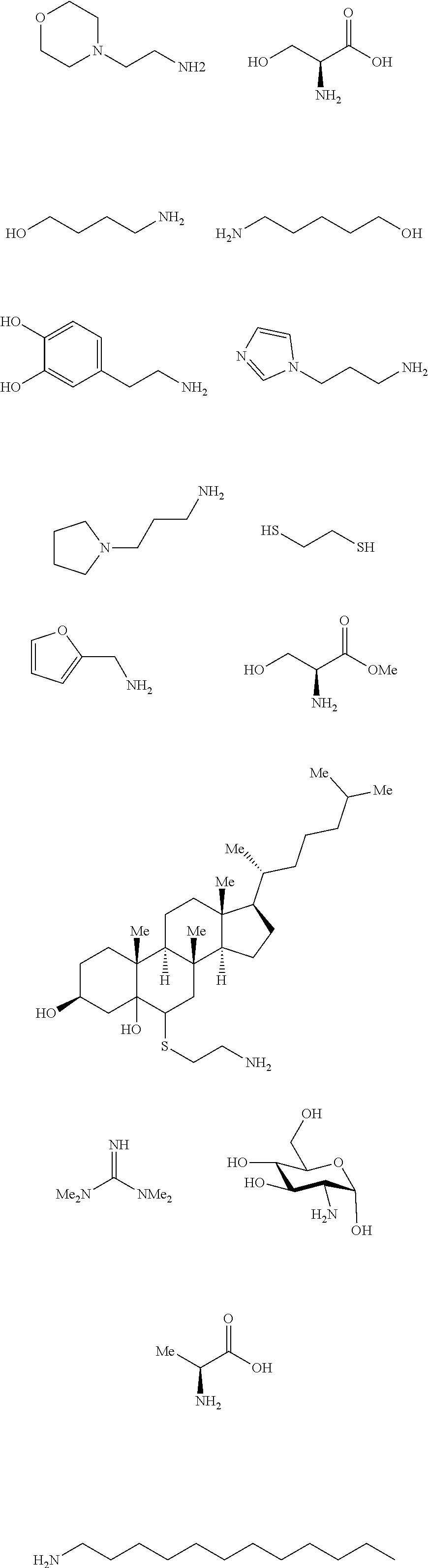

or any combination thereof.

In certain embodiments, Formula (B) comprises

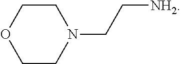

In certain embodiments, Formula (B) comprises

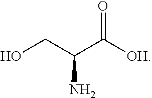

In certain embodiments, Formula (B) comprises

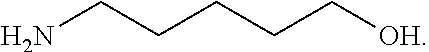

In certain embodiments, Formula (B) comprises

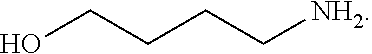

In certain embodiments, Formula (B) comprises

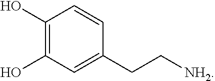 [SMILES: NCCc1ccc(O)c(O)c1]

In certain embodiments, Formula (B) comprises

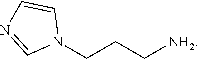

In certain embodiments, Formula (B) comprises

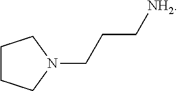

In certain embodiments, Formula (B) comprises

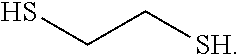

In certain embodiments, Formula (B) comprises

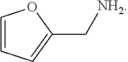

In certain embodiments, Formula (B) comprises

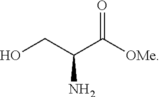

In certain embodiments, Formula (B) comprises

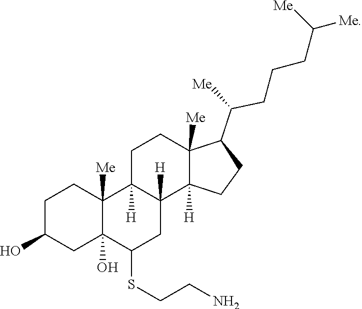

In certain embodiments, Formula (B) comprises

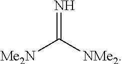

In certain embodiments, Formula (B) comprises

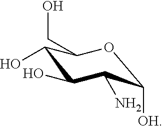

In certain embodiments, Formula (B) comprises

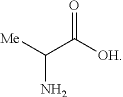

In certain embodiments, Formula (B) comprises

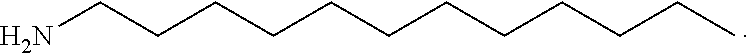

In certain embodiments, Formula (B) comprises a combination of any one of the above compounds of Formula (B). In certain embodiments, Formula (B) comprises a combination of two of the above compounds of Formula (B). In certain embodiments, Formula (B) comprises a combination of three of the above compounds of Formula (B). In certain embodiments Formula (B) comprises a combination of more than three of the above compounds of Formula (B). In any of the embodiments described, the components of Formula (B) may be present in any relative ratio.

In certain embodiments, Formula (B) comprises a combination of a dithiol and an amine in a ratio of 99:1 to 1:99. In certain embodiments, Formula (B) comprises a combination of a dithiol and an amine in a ratio of 95:5 to 5:95. In certain embodiments, Formula (B) comprises a combination of a dithiol and an amine in a ratio of 90:10 to 10:90. In certain embodiments, Formula (B) comprises a combination of a dithiol and an amine in a ratio of 85:15 to 15:85. In certain embodiments, Formula (B) comprises a combination of a dithiol and an amine in a ratio of 80:20 to 20:80. In certain embodiments, Formula (B) comprises a combination of a dithiol and an amine in a ratio of 75:25 to 25:75. In certain embodiments, Formula (B) comprises a combination of a dithiol and an amine in a ratio of 70:30 to 30:70. In certain embodiments, Formula (B) comprises a combination of a dithiol and an amine in a ratio of 65:35 to 35:65. In certain embodiments, Formula (B) comprises a combination of a dithiol and an amine in a ratio of 60:40 to 40:60. In certain embodiments, Formula (B) comprises a combination of a dithiol and an amine in a ratio of 55:45 to 45:55. In certain embodiments, Formula (B) comprises a combination of a dithiol and an amine in a ratio of 50:50.

In certain embodiments, Formula (B) comprises a combination of 1,2-ethanedithiol and 2-morpholinoethan-1-amine in a ratio of 99:1 to 1:99. In certain embodiments, Formula (B) comprises a combination 1,2-ethanedithiol and 2-morpholinoethan-1-amine in a ratio of 99:1 to 1:99. In certain embodiments, Formula (B) comprises a combination of 1,2-ethanedithiol and 2-morpholinoethan-1-amine in a ratio of 95:5 to 5:95. In certain embodiments, Formula (B) comprises a combination of 1,2-ethanedithiol and 2-morpholinoethan-1-amine in a ratio of 90:10 to 10:90. In certain embodiments, Formula (B) comprises a combination of 1,2-ethanedithiol and 2-morpholinoethan-1-amine in a ratio of 85:15 to 15:85. In certain embodiments, Formula (B) comprises a combination of 1,2-ethanedithiol and 2-morpholinoethan-1-amine in a ratio of 80:20 to 20:80. In certain embodiments, Formula (B) comprises a combination of 1,2-ethanedithiol and 2-morpholinoethan-1-amine in a ratio of 75:25 to 25:75. In certain embodiments, Formula (B) comprises a combination of 1,2-ethanedithiol and 2-morpholinoethan-1-amine in a ratio of 70:30 to 30:70. In certain embodiments, Formula (B) comprises a combination of 1,2-ethanedithiol and 2-morpholinoethan-1-amine in a ratio of 65:35 to 35:65. In certain embodiments, Formula (B) comprises a combination of 1,2-ethanedithiol and 2-morpholinoethan-1-amine in a ratio of 60:40 to 40:60. In certain embodiments, Formula (B) comprises a combination of 1,2-ethanedithiol and 2-morpholinoethan-1-amine in a ratio of 55:45 to 45:55. In certain embodiments, Formula (B) comprises a combination of 1,2-ethanedithiol and 2-morpholinoethan-1-amine in a ratio of 50:50.

In certain embodiments, Formula (B) comprises a combination of 1,2-ethanedithiol and 2-morpholinoethan-1-amine in a 1:4 ratio (a 0.1 to 0.4 ratio relative to Formula (A).

### Formula (C)

Formula (C) is an optional branching group having at least three (≥3) points of attachment to radicals of Formula (A). Triradicals of Formula (C) are branch points in the BAE/BTE-containing polymers of the invention. In certain embodiments, the polymers of the invention do not include a branch point of Formula (C).

In certain embodiments, Formula (C) is absent.

In certain embodiments, the polymer of Formula (I) comprises about 0 to 0.9 eq of Formula (C). In certain embodiments, the polymer of Formula (I) comprises about 0.1 to 0.4 eq of Formula (C).

In certain embodiments, Formula (C) comprises:

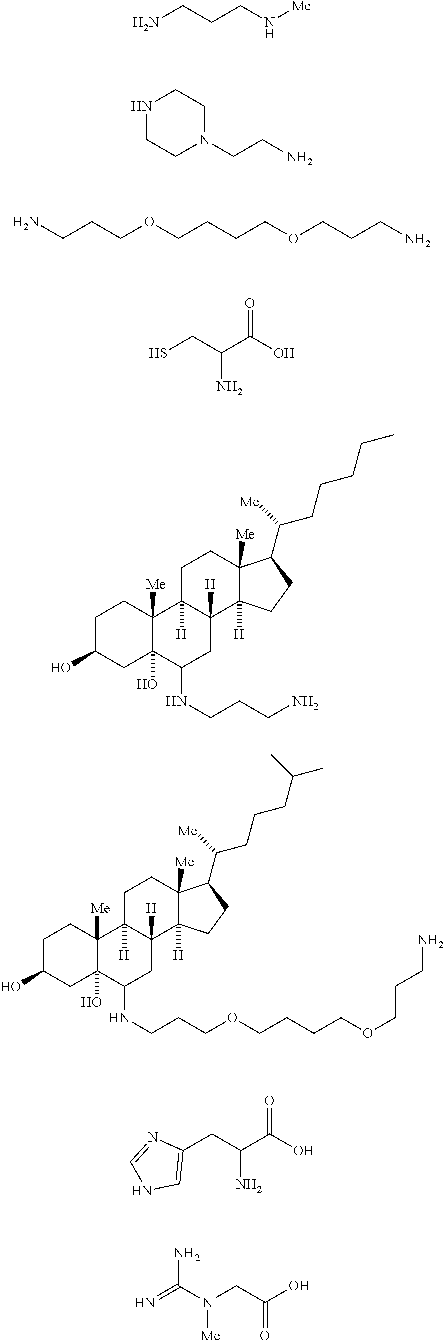

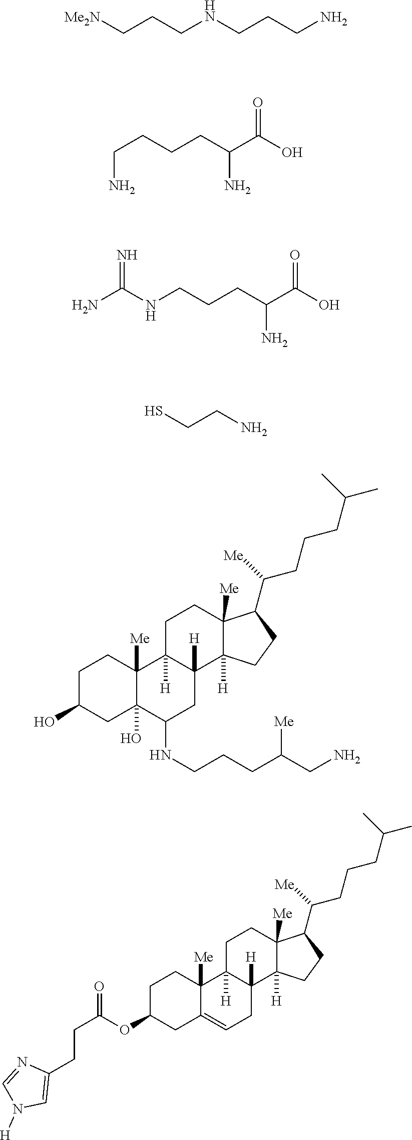

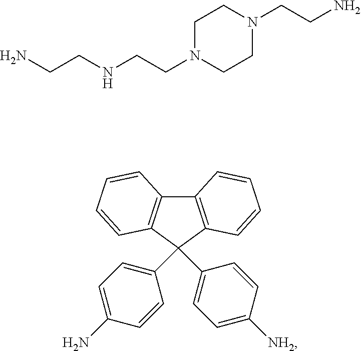 [SMILES: NCCNCCN1CCN(CCN)CC1 ; Nc1ccc(C2(c3ccc(N)cc3)c3ccccc3-c3ccccc32)cc1]

or any combination thereof.

In certain embodiments, Formula (C) comprises

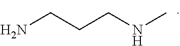

In certain embodiments, Formula (C) comprises

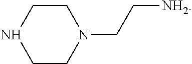

In certain embodiments, Formula (C) comprises

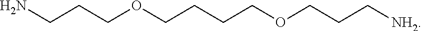 [SMILES: NCCCOCCCCOCCCN]

In certain embodiments, Formula (C) comprises

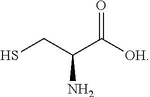

In certain embodiments, Formula (C) comprises

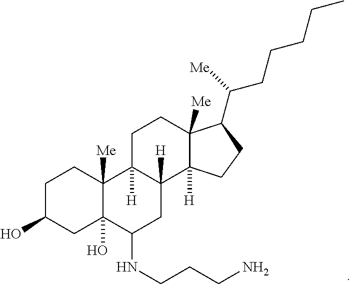

In certain embodiments, Formula (C) comprises

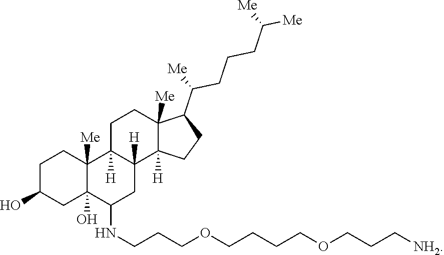

In certain embodiments, Formula (C) comprises

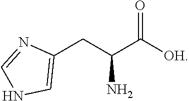 [SMILES: N[C@@H](Cc1c[nH]cn1)C(=O)O]

In certain embodiments, Formula (C) comprises

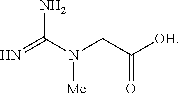

In certain embodiments, Formula (C) comprises

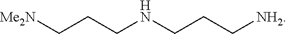 [SMILES: CN(C)CCCNCCCN]

In certain embodiments, Formula (C) comprises

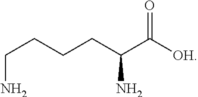

In certain embodiments, Formula (C) comprises

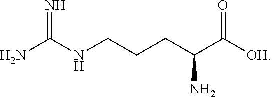 [SMILES: N=C(N)NCCC[C@H](N)C(=O)O]

In certain embodiments, Formula (C) comprises

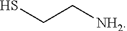

In certain embodiments, Formula (C) comprises

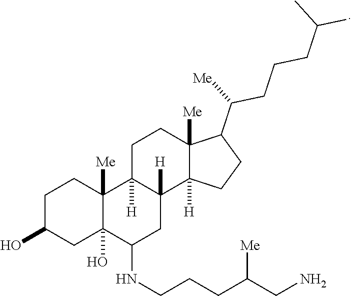

In certain embodiments, Formula (C) comprises

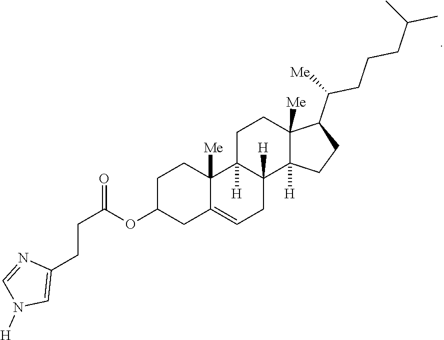

In certain embodiments, Formula (C) comprises

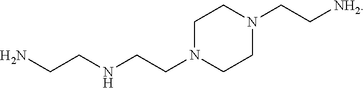 [SMILES: NCCNCCN1CCN(CCN)CC1]

In certain embodiments, Formula (C) comprises

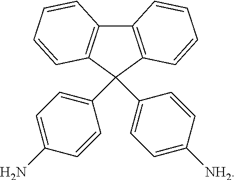 [SMILES: Nc1ccc(C2(c3ccc(N)cc3)c3ccccc3-c3ccccc32)cc1]

In certain embodiments, Formula (C) comprises a combination of any one of the above compounds of Formula (C). In certain embodiments, Formula (C) comprises a combination of two of the above compounds of Formula (C). In certain embodiments, Formula (C) comprises a combination of three of the above compounds of Formula (C). In certain embodiments Formula (C) comprises a combination of more than three of the above compounds of Formula (C). In all embodiments described, the components of Formula (C) may be present in any relative ratio.

### Formula (D)

Formula (D) is an optional end-capping group having one point of attachment to a radical of Formula (A). Radicals of Formula (D) occur at the termini of BAE/BTE-containing polymers of the invention.

In certain embodiments, Formula (D) is absent.

In certain embodiments, the polymer comprises about 0.5 to 2.0 eq Formula (D). In certain embodiments, the polymer comprises about 0.8 to 1.8 eq Formula (D).

In certain embodiments, Formula (D) comprises:

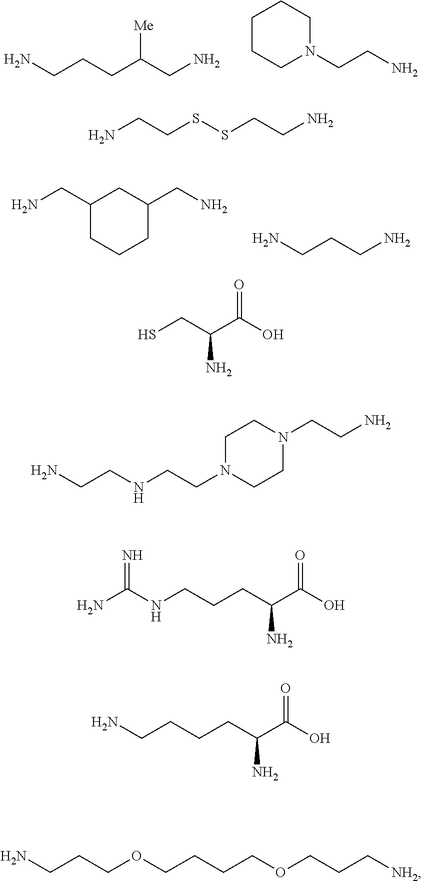

or combinations thereof.

In certain embodiments, Formula (D) comprises

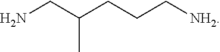

In certain embodiments, Formula (D) comprises

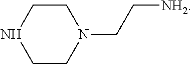

In certain embodiments, Formula (D) comprises

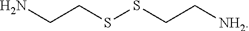

In certain embodiments, Formula (D) comprises

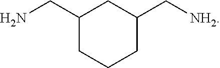

In certain embodiments, Formula (D) comprises

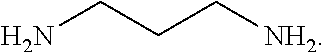

In certain embodiments, Formula (D) comprises

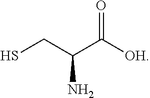

In certain embodiments, Formula (D) comprises

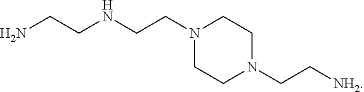 [SMILES: NCCNCCN1CCN(CCN)CC1]

In certain embodiments, Formula (D) comprises

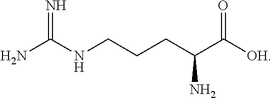 [SMILES: N=C(N)NCCC[C@H](N)C(=O)O]

In certain embodiments, Formula (D) comprises

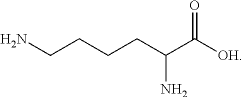

In certain embodiments, Formula (D) comprises

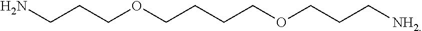 [SMILES: NCCCOCCCCOCCCN]

In certain embodiments, Formula (D) comprises a combination of any one of the above compounds of Formula (D). In certain embodiments, Formula (D) comprises a combination of two of the above compounds of Formula (D). In certain embodiments, Formula (D) comprises a combination of three of the above compounds of Formula (D). In certain embodiments Formula (D) comprises a combination of more than three of the above compounds of Formula (D). In all embodiments described, the components of Formula (D) may be present in any relative ratio.

### Exemplary Polymers of Formula (I)

Shown below are particularly preferred backbones of Formula (A):

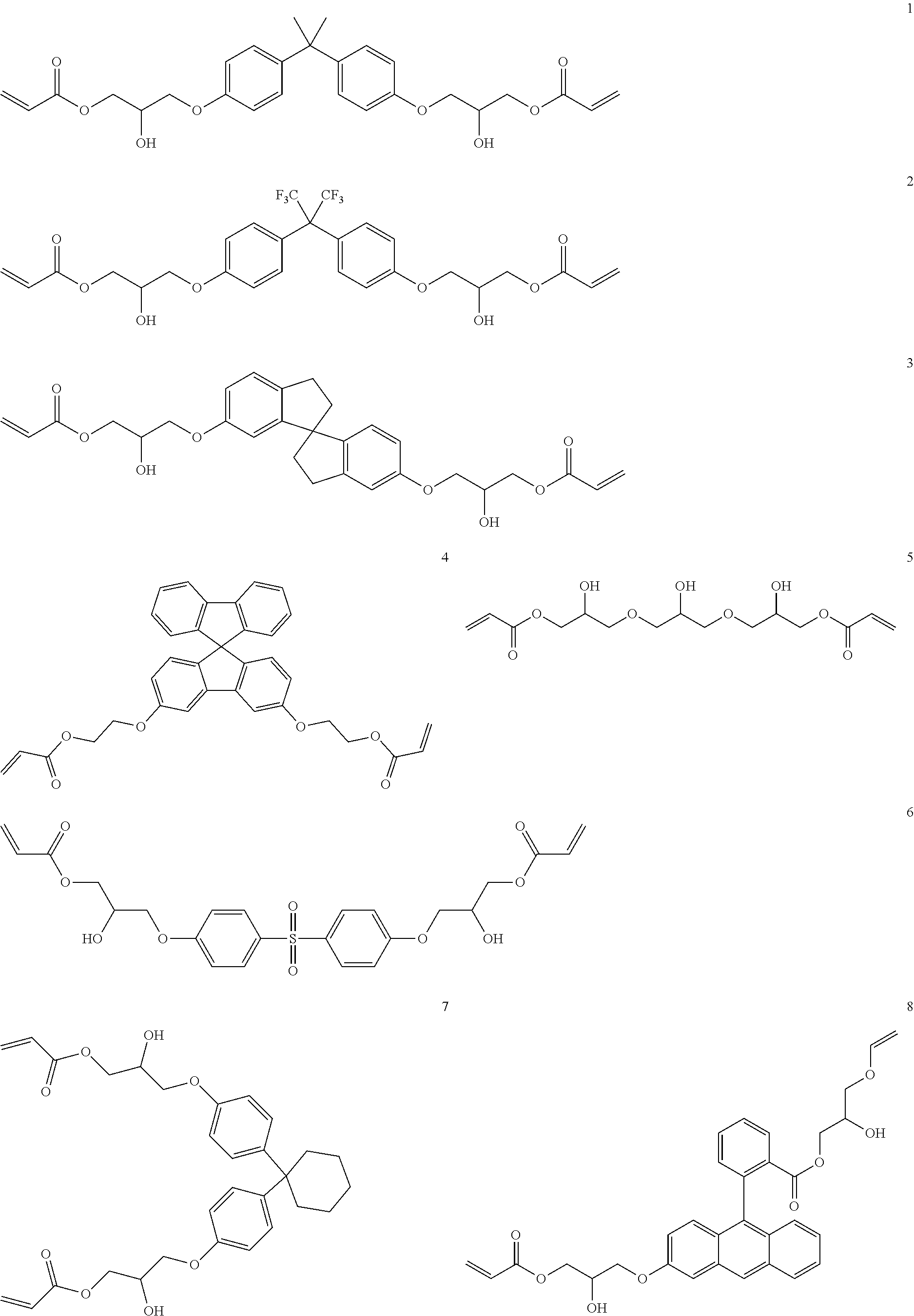

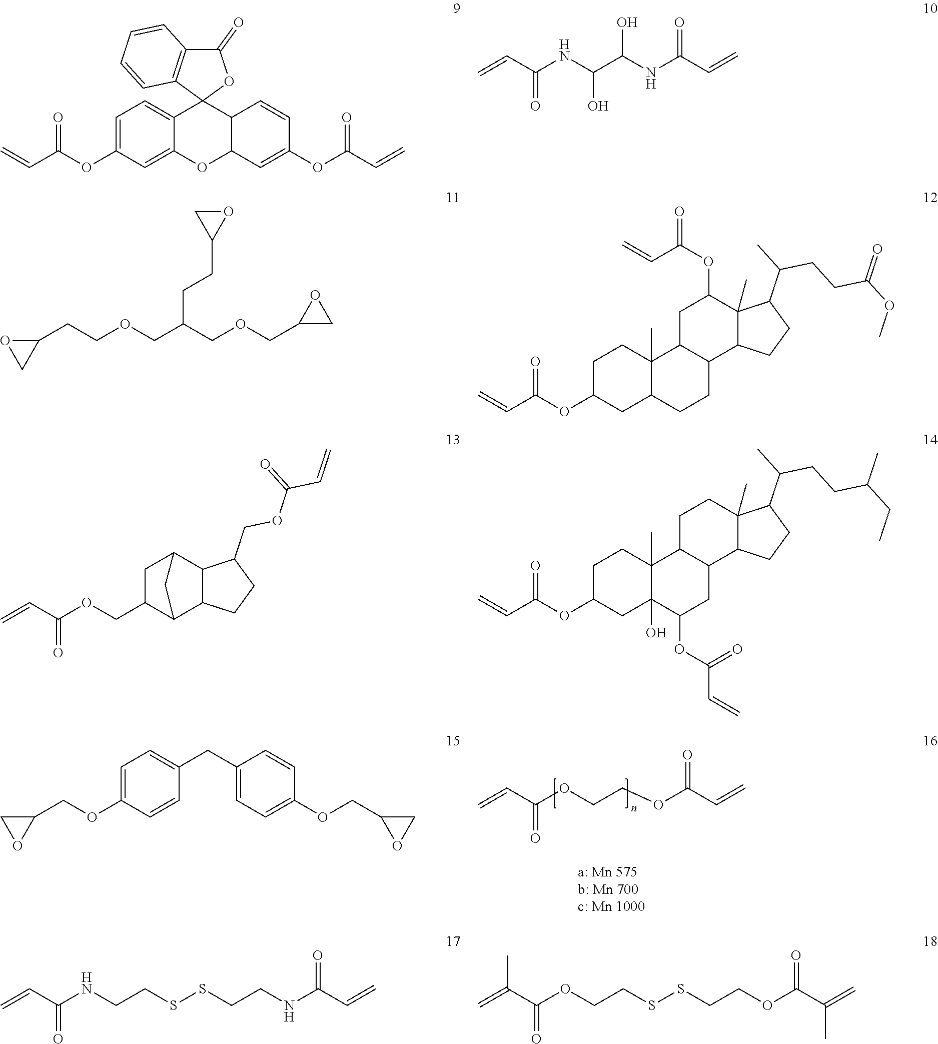

Shown below are particularly preferred linking groups of Formula (B):

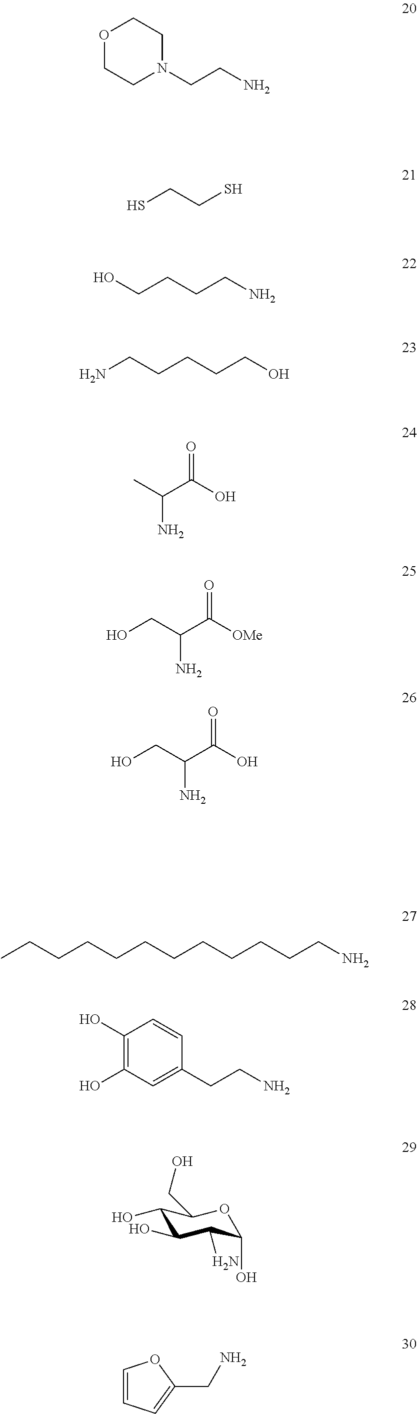

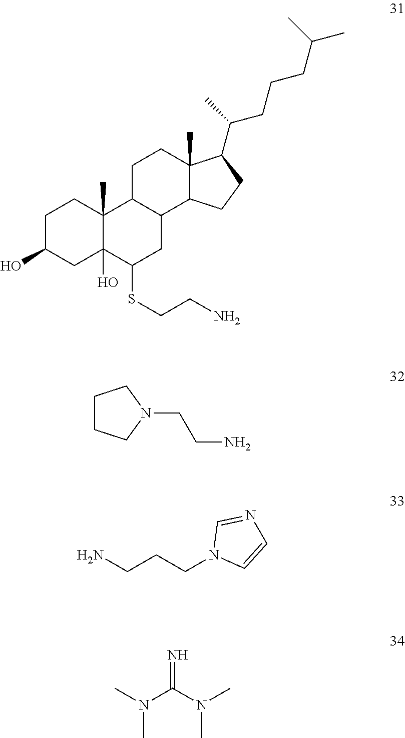

Shown below are particularly preferred branching groups of Formula (C):

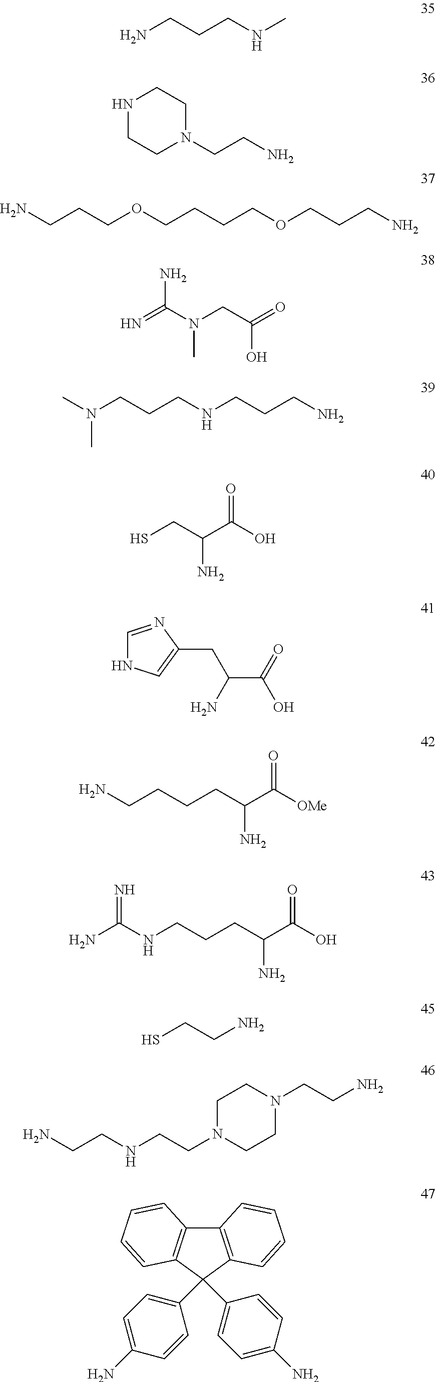

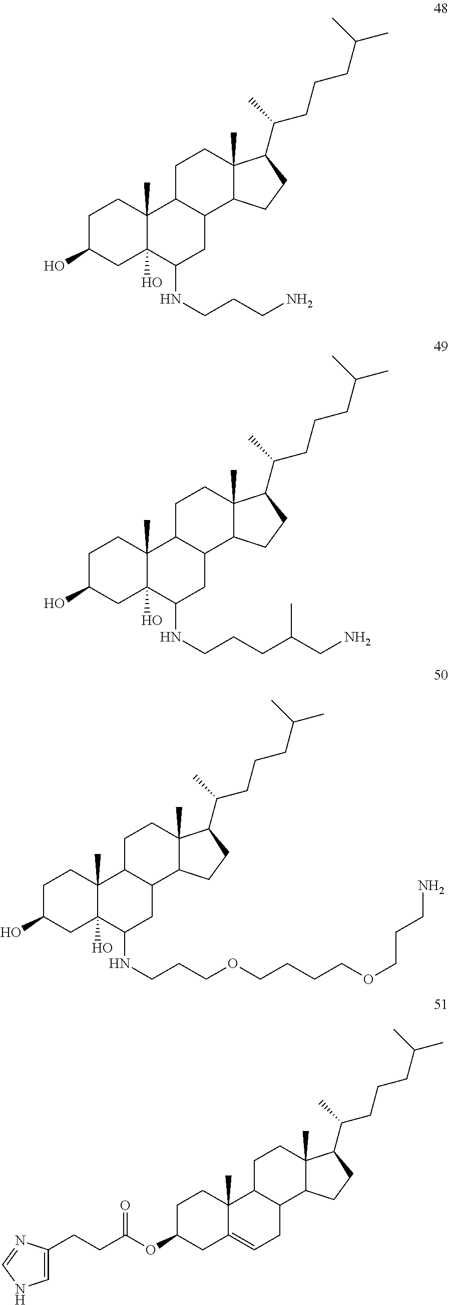

Shown below are particularly preferred end-capping groups of Formula (D):

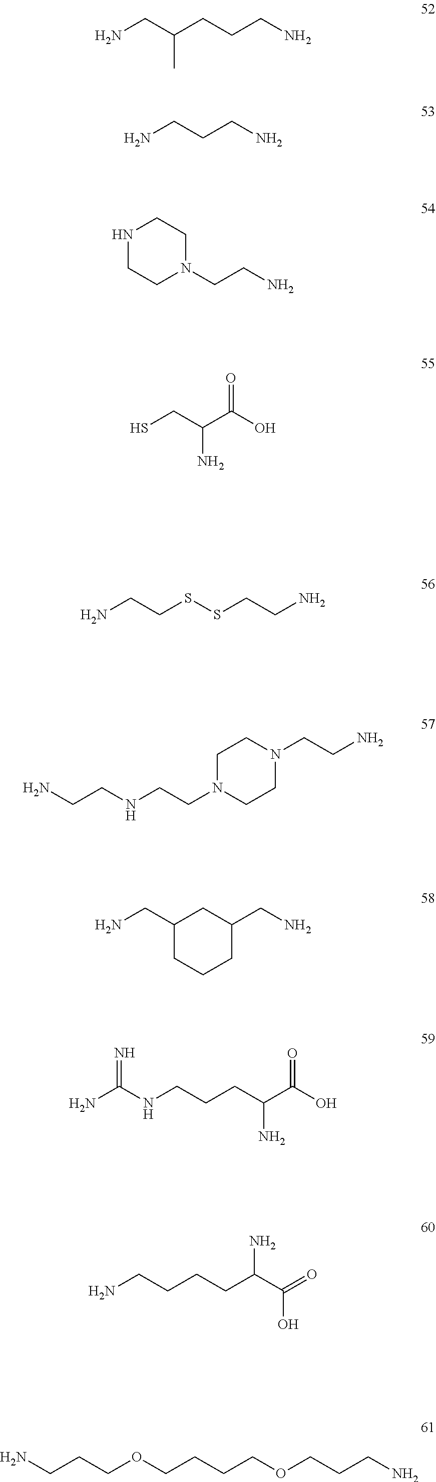

In certain particular embodiments. the polymers of Formula (I) are selected from any one of the compounds A, B, C, H, or 1-166 as shown in Table 2 below.

TABLE 2
Polymers of Formula (I)

| | | Branching | Linking | End-capping |
|---|---|---|---|---|
| Polymer of | Backbone of | Group of | Group of | Group of |
| Formula (I) | Formula (A) | Formula (B) | Formula (C) | Formula (D) |
| A | 1 | 43 | 20 | 52 |
| B | 4 | 21 | 20 | 52 |
| C | 18 | 21 | 20 | 57 |
| H | 18 | 21 | 20 | 52 |
| 1 | 4 (0.5) | 21 | 20 | 52 |
| | 5 (0.5) | | | |
| 2 | 13 (0.7) | 21 | 20 | 52 |
| | 5 (0.3) | | | |
| 3 | 13 (0.5) | 21 | 20 | 52 |
| | 5 (0.5) | | | |
| 4 | 13 | 21 | 20 | 52 |
| 5 | 5 | 32 | 44 | 53 |
| 6 | 5 | 41 | 22 | 53 |
| 7 | 9 | 21 | 20 | 52 |
| 8 | 1 | 43 | 20 | 52 |
| 9 | 3 | 21 | 20 | 52 |
| 10 | 1 | 47 | 20 | 21 |
| 11 | 3 (0.5) | 32 | 20 | 54 |
| | 5 (0.5) | | | |
| 12 | 3 (0.95) | 21 | 20 | 52 |
| | 10 (0.05) | | | |
| 13 | 3 | 21 | 20 | 52 |
| 14 | 3 (0.5) | 32 | 20 | 54 |
| | 5 (0.5) | | | |
| 15 | 3 (0.95) | 21 | 20 | 52 |
| | 10 (0.05) | | | |
| 16 | 3 | 41 | 22 | 52 |
| 17 | 17 | 32 | 44 | 52 |
| 18 | 17 | 46 | 44 | 61 |
| 19 | 1 (0.7) | 30 | 20 | 54 |
| | 18 (0.3) | | | |
| 20 | 1 (0.7) | 21 | 20 | 54 |
| | 17 (0.3) | | | |
| 21 | 9 (0.5) | 21 | 20 | 54 |
| | 5 (0.5) | | | |
| 22 | 9 (0.5) | 21 | 20 | 52 |
| | 10 (0.5) | | | |
| 23 | 1 (0.7) | 30 | 20 | 54 |
| | 10 (0.3) | | | |
| 24 | 1 (0.5) | 21 | 20 | 57 |
| | 10 (0.5) | | | |
| 25 | 13 (0.7) | 42 | 20 | 52 |
| | 5 (0.3) | | | |
| 26 | 13 (0.7) | 30 | 20 | 52 |
| | 5 (0.3) | | | |
| 27 | 13 (0.7) | 21 | 20 | 54 |
| | 5 (0.3) | | | |
| 28 | 13 (0.7) | 21 | 28 | 52 |
| | 5 (0.3) | | | |
| 29 | 13 (0.7) | 42 | 20 | 52 |
| | 10 (0.3) | | | |
| 30 | 13 (0.7) | 21 | 20 | 52 |
| | 10 (0.3) | | | |
| 31 | 13 (0.7) | 32 | 20 | 52 |
| | 10 (0.3) | | | |
| 32 | 19 (0.7) | 21 | 20 | 52 |
| | 10 (0.3) | | | |
| 33 | 1 | 21 | 20 | 54 |
| 34 | 1 | 42 | 20 | 52 |
| 35 | 1 | 21 | 23 | 52 |
| 36 | 1 | 40 | 20 | 52 |
| 37 | 1 | 39 | 20 | 52 |
| 38 | 1 | — | 20 | 54 |
| 39 | 13 (0.7) | 21 | 23 | 52 |
| | 5 (0.3) | | | |
| 40 | 13 (0.7) | 40 | 20 | 52 |
| | 5 (0.3) | | | |
| 41 | 13 (0.7) | 39 | 20 | 52 |
| | 5 (0.3) | | | |
| 46 | 13 | Amino acid mixture* | | 52 |
| 47 | 3 | 21 | 20 | 52 |
| 48 | 3 (0.5) | 21 | 20 | 54 |
| | 5 (0.5) | | | |
| 49 | 3 | 42 | 23 | 52 |
| 50 | 3 | 39 | 28 | 52 |
| 51 | 2 | 21 | 20 | 52 |
| 52 | 2 | 34 | 28 | 56 |
| 53 | 2 (0.5) | 21 | 20 | 52 |
| | 10 (0.5) | | | |
| 54 | 2 | 34 | 27 | 60 |
| 55 | 7 | 21 | 20 | 52 |
| 56 | 7 | 34 | 27 | 56 |
| 57 | 7 | 21 | 20 | 52 |
| | 10 | | | |
| 58 | 7 | 38 | 27 | 58 |
| 59 | 6 | 21 | 20 | 52 |
| 60 | 6 | 35 | 28 | 56 |
| 61 | 6 (0.8) | 21 | 20 | 52 |
| | 10 (0.2) | | | |
| 62 | 6 | 39 | 23 | 28 |
| 63 | 13 (0.7) | Amino acid mixture* | | 56 |
| | 5 (0.3) | | | |
| 64 | 9 (0.3) | Amino acid mixture* | | 56 |
| | 5 (0.7) | | | |
| 65 | 9 (0.3) | Amino acid | 56 | 52 |
| | 10 (0.7) | mixture* | | |
| 66* | 1 | 21 | 20 | 52 |
| 67 | 13 (0.7) | Amino acid | 52 | 52 |
| | 5 (0.3) | mixture* | | |
| 68 | 1 | Amino acid | 52 | 52 |
| | | mixture* | | |
| 69 | 1 (0.5) | 21 | 20 | 52 |
| | 10 (0.5) | | | |
| 70 | 1 (0.5) | — | 20 | 52 |
| | 10 (0.5) | | | |
| 71 | 13 (0.7) | 20 | 52 | 52 |
| | 5 (0.3) | | | |
| 72 | 1 (0.95) | 21 | 20 | 52 |
| | 11 (0.05) | | | |
| 73 | 1 (0.95) | 21 | 20 | 52 |
| | 15 (0.05) | | | |
| 74 | 1 (0.95) | 21 | 20 | 52 |
| | 17 (0.05) | | | |
| 75 | 1 | 20 | 52 | 52 |
| 76 | 1 | 31 (0.1) | 20 | 52 |
| | | 21 (0.4) | | |
| 77 | 1 | 21 | 26 (0.1) | 52 |
| | | | 20 (0.1) | |
| 78 | 1 | 21 | 29 (0.1) | 52 |
| | | | 20 (0.1) | |
| 79 | 3 | — | 29 | 52 |
| 80 | 13 (0.7) | — | 54 | 56 |
| | 5 (0.3) | | | |
| 81 | 13 (0.7) | — | 23 | 54 |
| | 5 (0.3) | | | |
| 82 | 1 | — | 28 | 52 |
| 83 | 3 | — | 20 | 52 |
| 84 | 3 | — | 26 | 56 |
| 85 | 3 | — | 29 | 59 |
| 86 | 1 | — | 24 | 52 |
| 87 | 1 | 36 | 24 | 52 |
| 88 | 1 | — | 29 | 60 |
| 89 | 8 | — | 20 | 54 |
| 90 | 8 | 36 | 24 | 52 |
| 91 | 8 | — | 29 | 59 |
| 92 | 1 (0.7) | — | 20 | 54 |
| | 10 (0.3) | | | |
| 93 | 1 | — | 29 | 56 |
| 94 | 1 | 36 | 29 | 52 |
| 95 | 1 | — | 25 | 24 |
| 96 | 1 | — | 29 | 24 |
| 97 | 1 | — | 20 | 54 |
| 98^{b} | 1 | 21 | 20 | 54 |
| 99 | 5 | — | 45 | 54 |
| 100 | 5 | — | 46 | 54 |
| 101 | 5 | — | 48 | 54 |
| 102 | 5 | — | 49 | 54 |
| 103 | 10 (0.5) | — | 45 | 54 |
| | 1 (0.5) | | | |
| 104 | 10 (0.7) | — | 45 | 54 |
| | 1 (0.3) | | | |
| 105 | 10 (0.5) | — | 45 | 54 |
| | 1 (0.7) | | | |
| 106 | 8 | — | 20 | 54 |
| 107 | 1 | — | 20 | 54 |
| 108 | 10 (0.5) | 46 (0.2) | | 54 |
| | 1 (0.5) | 24 (0.2) | | |
| | | 36 (0.2) | | |
| 109 | 110 (0.5) | 46 (0.2) | | 54 |
| | 1 (0.5) | 24 (0.2) | | |
| | | 36 (0.2) | | |
| 110 | 5 | — | 46 (0.8) | — |
| 111 | 1 | — | 29 | 54 |
| 112 | 1 | 20 | 55 | 112 |
| 113 | 10 (0.5) | — | 48 | 54 |
| | 1 (0.5) | | | |
| 114 | 10 (0.7) | — | 48 | 54 |
| | 1 (0.3) | | | |
| 115 | 10 (0.3) | — | 48 | 54 |
| | 1 (0.7) | | | |
| 116 | 1 | — | 36 | 52 |
| 117 | 14 (0.7) | — | 24 | 52 |
| | 5 (0.3) | | | |
| 118 | 14 (0.7) | 36 | 24 | 52 |
| | 5 (0.3) | | | |
| 119 | 14 (0.7) | — | 20 | 54 |
| | 5 (0.3) | | | |
| 120 | 14 (0.7) | — | 37 | 52 |
| | 5 (0.3) | | | |
| 121 | 14 (0.7) | 36 | 24 | 52 |
| | 5 (0.3) | | | |
| 122 | 14 (0.7) | — | 24 | 52 |
| | 5 (0.3) | | | |
| 123 | 14 (0.7) | 36 | 24 | 52 |
| | 5 (0.3) | | | |
| 124 | 14 (0.7) | 20 | 54 | 52 |
| | 5 (0.3) | | | |
| 126 | 14 (0.7) | — | 37 | 52 |
| | 5 (0.3) | | | |
| 127 | 10 (0.5) | 45 (0.2) | | 54 |
| | 14 (0.5) | 36 (0.2) | | |
| | | 24 (0.2) | | |
| 128 | 14 (0.5) | 46 (0.2) | | 54 |
| | 10 (0.5) | 36 (0.2) | | |
| | | 24 (0.2) | | |
| 129 | 5 | 45 | 29 | — |
| 130 | 5 | 45 | 28 | — |
| 131 | 5 | 46 | 29 | — |
| 132 | 5 | 46 | 28 | — |
| 133 | 5 | 48 | 29 | — |
| 134 | 5 | 48 | 28 | — |
| 135 | 5 | 50 | 29 | — |
| 136 | 5 | 50 | 54 | — |
| 137 | 5 | 36 | 48 | 54 |
| 138 | 5 | — | 46 | 54 |
| 139 | 5 (0.7) | — | 24 | 52 |
| | 8 (0.3) | | | |
| 140 | 5 (0.7) | 36 | 55 | 52 |
| | 8 (0.3) | | | |
| 142 | 16^{b} | 21 | 52 | 52 |
| 143 | 1 (0.95) | 21 | 52 | 52 |
| | 16^{b} (0.05) | | | |
| 144 | 1 (0.98) | 20 | 50 | 52 |
| | 16^{b} (0.02) | | | |
| 145 | 16^{b} (0.5) | 21 | 45 | 52 |
| | 1 (0.5) | | | |
| 146 | 16^{a} (0.2) | 21 | 46 | 52 |
| | 1 (0.8) | | | |
| 147 | 16^{b} (0.5) | 21 | 47 | 52 |
| | 1 (0.5) | | | |
| 148 | 16^{b} | 46 | — | — |
| 149 | 16^{c} | 30 | 46 | 52 |
| 150 | 16^{c} | 48 | 46 | 52 |
| 151 | 1 (0.2) | 21 | 48 | 54 |
| | 16^{b} (0.8) | | | |
| 152 | 1 (0.2) | 21 | 48 | 54 |
| | 16^{a} (0.8) | | | |
| 153 | 16^{a} | 31 (0.4) | 48 | — |
| 154 | 16^{b} | 31 (0.4) | 48 | — |
| 155 | 1 (0.5) | 31 | 48 | 54 |
| | 16^{b} (0.5) | | | |
| 156 | 1 (0.5) | 31 | 48 | 54 |
| | 16^{a} (0.5) | | | |
| 157 | 16^{b} | 48 | — | 54 |
| 158 | 16^{a} | 48 | — | 54 |
| 159 | 1 (0.5) | 31 | 20 | 54 |
| | 16^{b} (0.5) | | | |
| 160 | 1 (0.5) | 31 | 20 | 54 |
| | 16^{a} (0.5) | | | |
| 161 | 1 | 31 | 20 | 54 |
| 162^{c} | 1 | 21 | 20 | 52 |
| 163 | 1 | 31 | — | 28 |
| 164 | 1 | 31 | 20 | 28 |
| 165 | 1 | 36 (0.4) | 29 | 28 |
| | | 31 (0.1) | | |
| 166 | 1 | — | 29 | 52 |

Regular ratios: 1:0.5:0.2:1.5;
*beta-alanine, citrulline, creatine
^{a}modified workup;
^{b}run in DMPU;
^{c}run in ¹³C-DMF

### Methods of Preparation

Provided herein are methods of preparing a polymer of Formula (I), comprising:
(i) combining a backbone of Formula (A) with a linking group of Formula (B) and a branching group of Formula (C); and
(ii) combining the product of step (i) with a compound of Formula (D) such that a polymer of Formula (I) is formed.

Also provided herein are a polymers of Formula (I) that are prepared according to a method described herein, for example, in Examples 1 and 2.

In certain embodiments, the monomers are reacted at temperatures ranging from 25° C. to 120° C. for 24 to 72 hours. Monomers of Formulae (A), (B) and/or (D) may be added at the same time or in steps. In certain embodiments, monomers of Formulae (A) and (C) are reacted at 40° C. for 24 hours and then monomers of Formulae (B) and/or (D) added at 24 hours and reacted at 90° C. for a further 24-48 hours. In certain embodiments, all monomers are added at same time at a temp of 40° C. for 6 hours followed by an increase in temp to 90° C. and stirred up to 48 hours. In other embodiments, all monomers added at same time and reacted at 90° C. for 48 hours.

### Agents

mRNA encoding a nanoluciferase with encoded GPI anchor (1279 nt), called aNLuc; an immunoglobulin G (IgG) antibody (light chain mRNA length: 1,890 nt, heavy chain mRNA length with encoded GPI anchor: 2,180 nt), called aHCA, with no target in non-human species; LbuCas13a (4,719 nt); dCas9-VPR (˜7,055 nt). For both Cas13a and dCas9-VPR, we also included a short non-targeted CRISPR RNA (crRNA) of 60 nt for Cas13a and 100 nt for dCas9-VPR, to simulate a fully realized CRISPR drug. To assess IgG expression, we used an mRNA-encoded light chain fused to the NLuc protein (aHCA-Nluc), while for the Cas13a and dCas9-VPR constructs, the NLuc reporter sequence was downstream of an encoded 2A cleavage site. We also investigated the difference in expression using P76 between BALB/c and DBA/2 mice strains.

The agents to be delivered by polymers and compositions of the present invention may be therapeutic, diagnostic, or prophylactic agents. Any chemical compound to be administered to an individual may be delivered using the inventive polymers, compositions, complexes, picoparticles, nanoparticles, microparticles, micelles, or liposomes. The agent may be a nucleic acid, oligonucleotide, polynucleotide, drug, immunological agent, etc.

In certain embodiments, the agent to be delivered is a combination of agents.

### Polynucleotide

In certain embodiments, the polynucleotide is any type of RNA. In certain embodiments, the polynucleotide is mRNA, siRNA, SSRNA, dsRNA, shRNA, miRNA, circular RNA, circular mRNA, self-amplifying RNA, guide or CRISPR RNA. In certain particular embodiments, the polynucleotide is mRNA.

In certain embodiments, the polynucleotide is an RNA that carries out RNA interference (RNAi). The phenomenon of RNAi is discussed in greater detail, for example, in the following references, each of which is incorporated herein by reference: Elbashir et al., 2001, Genes Dev., 15:188; Fire et al., 1998, Nature, 391:806; Tabara et al., 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553.

In certain embodiments, the polynucleotide is a dsRNA (double-stranded RNA).

In certain embodiments, the polynucleotide is an siRNA (short interfering RNA).

In certain embodiments, the polynucleotide is an shRNA (short hairpin RNA).

In certain embodiments, the polynucleotide is an miRNA (micro RNA). Micro RNAs (miRNAs) are genomically encoded non-coding RNAs of about 21-23 nucleotides in length that help regulate gene expression, particularly during development (see, e.g., Bartel, 2004, Cell, 116:281; Novina and Sharp, 2004, Nature, 430:161; and U.S. Patent Publication 2005/0059005; also reviewed in Wang and Li, 2007, Front. Biosci., 12:3975; and Zhao, 2007, Trends Biochem. Sci., 32:189; each of which are incorporated herein by reference).

In certain embodiments, the polynucleotide is an antisense RNA.

In certain embodiments, the polynucleotide is a self-amplifying RNA (see Geall, 2012, PNAS, 109 (36) 14604-14609)

In certain embodiments, the polynucleotide is a circular RNA (see Wesselhoeft et al. (2018) 9:2629; Bogers et al. JID 2015:211; Yu and Kuo Journal of Biomedical Science (2019) 26:29),

In certain embodiments, the polynucleotide is a circular mRNA.

In some embodiments, an RNA can be designed and/or predicted using one or more of a large number of available algorithms. To give but a few examples, the following resources can be utilized to design and/or predict dsRNA, siRNA, shRNA and/or miRNA: algorithms found at Alnylum Online, Dharmacon Online, OligoEngine Online, Molecula Online, Ambion Online, BioPredsi Online, RNAi Web Online, Chang Bioscience Online, Invitrogen Online, LentiWeb Online GenScript Online, Protocol Online; Reynolds et al., 2004, Nat. Biotechnol., 22:326; Naito et al., 2006, Nucleic Acids Res., 34: W448; Li et al., 2007, RNA, 13:1765; Yiu et al., 2005, Bioinformatics, 21:144; and Jia et al., 2006, BMC Bioinformatics, 7:271; each of which is incorporated herein by reference).

The polynucleotides may be of any size or sequence, and they may be single-or double-stranded. In certain embodiments, the polynucleotide is greater than 100 base pairs long. In certain embodiments, the polynucleotide is greater than 1000 base pairs long and may be greater than 10,000 base pairs long. The polynucleotide is optionally purified and substantially pure. Preferably, the polynucleotide is greater than 50% pure, more preferably greater than 75% pure, and most preferably greater than 95% pure. The polynucleotide may be provided by any means known in the art. In certain embodiments, the polynucleotide has been engineered using recombinant techniques (for a more detailed description of these techniques, please see Ausubel et al. Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989); each of which is incorporated herein by reference). The polynucleotide may also be obtained from natural sources and purified from contaminating components found normally in nature. The polynucleotide may also be chemically synthesized in a laboratory. In certain embodiments, the polynucleotide is synthesized using standard solid phase chemistry.

The polynucleotides may also contain mixtures of different size sequences, such as an mRNA and a guide mRNA, or mRNA and siRNA or mRNA and antisense RNA.

The polynucleotide may be modified by chemical or biological means. In certain embodiments, these modifications lead to increased stability of the polynucleotide. Modifications include methylation, phosphorylation, end-capping, etc.

Derivatives of polynucleotides may also be used in the present invention. These derivatives include modifications in the bases, sugars, and/or phosphate linkages of the polynucleotide. Modified bases include, but are not limited to, those found in the following nucleoside analogs: pseudouridine, N1-methyl-pseudouridine, 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine. Modified sugars include, but are not limited to, 2′-fluororibose, ribose, 2′-deoxyribose, 3′-azido-2′,3′-dideoxyribose, 2′,3′-dideoxyribose, arabinose (the 2′-epimer of ribose), acyclic sugars, and hexoses. The nucleosides may be strung together by linkages other than the phosphodiester linkage found in naturally occurring DNA and RNA. Modified linkages include, but are not limited to, phosphorothioate and 5′-N-phosphoramidite linkages. Combinations of the various modifications may be used in a single polynucleotide. These modified polynucleotides may be provided by any means known in the art; however, as will be appreciated by those of skill in this art, the modified polynucleotides are preferably prepared using synthetic chemistry in vitro.

The polynucleotides to be delivered may be in any form. For example, the polynucleotide may be a circular plasmid, a linearized plasmid, a cosmid, a viral genome, a modified viral genome, an artificial chromosome, a linear or circularized messenger RNA, self-amplifying RNA, a guide or CRISPR RNA, etc.

The polynucleotide may be of any sequence. In certain embodiments, the polynucleotide encodes a protein or peptide. The encoded proteins may be enzymes, structural proteins, receptors, soluble receptors, ion channels, pharmaceutically active proteins, cytokines, interleukins, antibodies, antibody fragments, antigens, coagulation factors, albumin, growth factors, hormones, insulin, etc. The polynucleotide may also comprise regulatory regions to control the expression of a gene. These regulatory regions may include, but are not limited to, promoters, enhancer elements, repressor elements, TATA box, ribosomal binding sites, stop site for transcription, etc. In certain embodiments, the polynucleotide is not intended to encode a protein. For example, the polynucleotide may be used to fix an error in the genome of the cell being transfected.

The polynucleotide may also be provided as an antisense agent or RNA interference (RNAi) agent (Fire et al. Nature 391:806-811, 1998; incorporated herein by reference). Antisense therapy is meant to include, e.g., administration or in situ provision of single- or double-stranded oligonucleotides or their derivatives which specifically hybridize, e.g., bind, under cellular conditions, with cellular mRNA and/or genomic DNA, or mutants thereof, so as to inhibit expression of the encoded protein, e.g., by inhibiting transcription and/or translation (Crooke “Molecular mechanisms of action of antisense drugs” Biochim. Biophys. Acta 1489 (1): 31-44, 1999; Crooke “Evaluating the mechanism of action of antiproliferative antisense drugs” Antisense Nucleic Acid Drug Dev. 10 (2): 123-126, discussion 127, 2000; Methods in Enzymology volumes 313-314, 1999; cach of which is incorporated herein by reference). The binding may be by conventional base pair complementarity, or, for example, in the case of binding to DNA duplexes, through specific interactions in the major groove of the double helix (i.e., triple helix formation) (Chan et al. J. Mol. Med. 75 (4): 267-282, 1997; incorporated herein by reference).

In certain embodiments, the polynucleotide to be delivered comprises a sequence encoding an antigenic peptide or protein. Nanoparticles containing these polynucleotides can be delivered to an individual to induce an immunologic response sufficient to decrease the chance of a subsequent infection and/or lessen the symptoms associated with such an infection. The polynucleotide of these vaccines may be combined with interleukins, interferon, cytokines, and adjuvants such as cholera toxin, alum, Freund's adjuvant, etc. A large number of adjuvant compounds are known; a useful compendium of many such compounds is prepared by the National Institutes of Health and can be found on the internet (www.niaid.nih.gov/daids/vaccine/pdf/compendium.pdf, incorporated herein by reference; see also Allison Dev. Biol. Stand. 92:3-11, 1998; Unkeless et al. Annu. Rev. Immunol. 6:251-281, 1998; and Phillips et al. Vaccine 10:151-158, 1992, each of which is incorporated herein by reference).

The antigenic protein or peptides encoded by the polynucleotide may be derived from such bacterial organisms as coronaviruses, Streptococcus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Hacmophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; from such viruses as smallpox, influenza A and B, respiratory syncytial virus A and B, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; and from such fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniac, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like.

The polymers of the present invention (e.g., polymers of Formula (I)) may comprise, primary, secondary, and tertiary amines. Although these amines are sterically hindered, they are available to interact with a polynucleotide (e.g., DNA, RNA, synthetic analogs of DNA and/or RNA, DNA/RNA hydrids, etc.). Polynucleotides or derivatives thereof are contacted with the polymers of the invention under conditions suitable to form polynucleotide complexes. The polymer of the invention is preferably at least partially protonated so as to form a complex with the negatively charged polynucleotide. The polymer of the invention makes hydrogen bonding between the oxygen atoms of the backbone and the bases of the mRNA, as well as π-π interactions between the backbone and the RNA bases. In some embodiments, the sulfur groups in the linker section interact with the RNA backbone, likely through hydrophobic interactions, making a more stable bond between the polymer and RNA cargo. In certain embodiments, the polynucleotide complexes form particles that are useful in the delivery of polynucleotides to cells. In certain embodiments, multiple molecules of a polymer of the invention may be associated with a polynucleotide molecule.

The polymers of the present invention are useful as drug delivery vehicles. The polymers may be used to encapsulate agents including polynucleotides, small molecules, proteins, peptides, metals, organometallic compounds, etc. The polymers have several properties that make them particularly suitable in the preparation of drug delivery vehicles. These include: 1) the ability of the polymer to complex and “protect” labile agents; 2) the ability to buffer the pH in the endosome; and/or 3) the ability to neutralize the charge on negatively charged agents. In certain embodiments, the polymers are used to form particles containing the agent to be delivered. These particles may include other materials, such as steroids (e.g., cholesterol), proteins, carbohydrates, synthetic polymers (e.g., PEG, PLGA), lipids, and natural polymers.

### Methods of Preparing Particles

Polymers according to the present disclosure may be used to prepare particles. Methods for preparing particles using the presently disclosed polymers include, but are not limited to, lyophilization, spray drying, single and double emulsion solvent evaporation, solvent extraction, phase separation, simple and complex coacervation, and other methods well known to those of ordinary skill in the art. In certain embodiments, methods of preparing the particles are the double emulsion process and spray drying. The conditions used in preparing the particles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283, 1987; Mathiowitz et al. J. Appl. Polymer Sci. 35:755-774, 1988; each of which is incorporated herein by reference).

If the particles prepared by any of the above methods have a size range outside of the desired range, the particles can be sized, for example, using a sieve. The particle may also be coated. In certain embodiments, the particles are coated with a targeting agent. In other embodiments, the particles are coated to achieve desirable surface properties (e.g., a particular charge).

### Compositions

The composition may comprise one type of polymer of the invention but may also comprise any number of different types, e.g., 1, 2, 3, 4, 5, 6, 7, 8, 9, 10 or more different types of polymers of the invention.

The composition may comprise an agent, as described herein. When the agent is a polynucleotide, the composition may be characterized in terms of an N/P ratio (i.e., the ratio of moles of the amine groups of the polymer of the invention to moles of the phosphate groups of the polynucleotide).

In certain embodiments, the composition is formulated for aerosol delivery. In certain embodiments, the composition is in the form of a particle.

### Targeting Agents

The inventive polymers, compositions, complexes, liposomes, micelles, microparticles, picoparticles, and nanoparticles may be modified to include targeting agents since it is often desirable to target a particular cell, collection of cells, or tissue. A variety of targeting agents that direct pharmaceutical compositions to particular cells are known in the art (see, for example, Cotten et al., Methods Enzym. 217:618, 1993; incorporated herein by reference). The targeting agents may be included throughout the particle or may be only on the surface. The targeting agent may be a protein, peptide, carbohydrate, glycoprotein, lipid, small molecule, nucleic acid, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, etc. If the targeting agent is included throughout the particle, the targeting agent may be included in the combination that is used to form the particles. If the targeting agent is only on the surface, the targeting agent may be associated with (i.e., by covalent, hydrophobic, hydrogen bonding, van der Waals, or other interactions) the formed particles using standard chemical techniques.

### Treatment Methods

It is estimated that over 10,000 human diseases are caused by genetic disorders, which are abnormalities in genes or chromosomes. See, e.g., McClellan, J. and M. C. King, Genetic heterogeneity in human disease. Cell. 141 (2): p. 210-7; Leachman, S. A., et al., Therapeutic siRNAs for dominant genetic skin disorders including pachyonychia congenita. J Dermatol Sci, 2008. 51 (3): p. 151-7. Many of these diseases are fatal, such as cancer, severe hypercholesterolemia, and familial amyloidotic polyneuropathy. See, e.g., Frank-Kamenetsky, M., et al., Therapeutic RNAi targeting PCSK9 acutely lowers plasma cholesterol in rodents and LDL cholesterol in nonhuman primates. Proc Natl Acad Sci USA, 2008. 105 (33): p. 11915-20; Coelho, T., Familial amyloid polyneuropathy: new developments in genetics and treatment. Curr Opin Neurol, 1996. 9 (5): p. 355-9. Since the discovery of gene expression silencing via RNA interference (RNAi) by Fire and Mello (Fire, A., et al., Potent and specific genetic interference by double-stranded RNA in Caenorhabditis clegans. Nature, 1998. 391 (6669): p. 806-11), there has been extensive effort toward developing therapeutic applications for RNAi in humans. See, e.g., Davis, M. E., The first targeted delivery of siRNA in humans via a self-assembling, cyclodextrin polymer-based nanoparticle: from concept to clinic. Mol Pharm, 2009. 6 (3): p. 659-68; Whitehead, K. A., R. Langer, and D. G. Anderson, Knocking down barriers: advances in siRNA delivery. Nat. Rev. Drug Discovery, 2009. 8 (2): p. 129-138; Tan, S. J., et al., Engineering Nanocarriers for siRNA Delivery. Small. 7 (7): p. 841-56; Castanotto, D. and J. J. Rossi, The promises and pitfalls of RNA-interference-based therapeutics. Nature, 2009. 457 (7228): p. 426-33; Chen, Y. and L. Huang, Tumor-targeted delivery of siRNA by non-viral vector: safe and effective cancer therapy. Expert Opin Drug Deliv, 2008. 5 (12): p. 1301-11; Weinstein, S. and D. Peer, RNAi nanomedicines: challenges and opportunities within the immune system. Nanotechnology. 21 (23): p. 232001; Fenske, D. B. and P. R. Cullis, Liposomal nanomedicines. Expert Opin Drug Deliv, 2008. 5 (1): p. 25-44; and Thiel, K. W. and P. H. Giangrande, Therapeutic applications of DNA and RNA aptamers. Oligonucleotides, 2009. 19 (3): p. 209-22. Currently, there are more than 20 clinical trials ongoing or completed involving siRNA therapeutics, which have shown promising results for the treatment of various diseases. See, e.g., Burnett, J. C., J. J. Rossi, and K. Tiemann, Current progress of siRNA/shRNA therapeutics in clinical trials. Biotechnol J. 6 (9): p. 1130-46. However, the efficient and safe delivery of siRNA is still a key challenge in the development of siRNA therapeutics. See, e.g., Juliano, R., et al., Biological barriers to therapy with antisense and siRNA oligonucleotides. Mol Pharm, 2009. 6 (3): p. 686-95.

Accordingly, provided herein are methods of using polymers of the invention, e.g., a polymer of Formula (I), for the treatment of a disease, disorder, or condition from which a subject suffers. It is contemplated that polymers of the invention will be useful in the treatment of a variety of diseases, disorders, or conditions, especially a system for delivering agents useful in the treatment of that particular disease, disorder, or condition. “Disease,” “disorder,” and “condition” are used interchangeably herein. In certain embodiments, the disease, disorder or condition from which a subject suffers is caused by an abnormality in a gene or chromosome of the subject.

For example, in one embodiment, provided is a method of treating disease, disorder, or condition from which a subject suffers, comprising administering to a subject in need thereof an effective amount of a composition comprising a polymer of the invention, e.g., a polymer of Formula (I), or salt thereof. Exemplary disease, disorder, or conditions contemplated include, but are not limited to, proliferative disorders, inflammatory disorders, autoimmune disorders, painful conditions, lung diseases, liver diseases, amyloid neuropathies, enzyme deficiencies and cystic fibrosis.

In certain particular embodiments, the method is for treating lung disease. In certain embodiments, the lung disease is asthma, chronic obstructive pulmonary disease (COPD), chronic bronchitis, emphysema, pulmonary hypertension, pulmonary fibrosis (e.g., idiopathic pulmonary fibrosis, fibrotic interstitial lung disease, interstitial pneumonia, fibrotic variant of non-specific interstitial pneumonia, or cystic fibrosis), sarcoidosis, influenza, pneumonia, tuberculosis, or lung cancer. In certain embodiments, the lung cancer is bronchogenic carcinoma, small cell lung cancer (SCLC), non-small cell lung cancer (NSCLC), or adenocarcinoma of the lung.

In certain embodiments, the composition further comprises, in addition to the polymer of the invention, a therapeutic agent useful in treating the disease, disorder, or condition. In certain embodiments, the polymer of the invention encapsulates the other (therapeutic) agent. In certain embodiments, the polymer of the invention and the other (therapeutic) agent form a particle (e.g., a nanoparticle, a microparticle, a micelle, a liposome, a lipoplex).

In certain embodiments, the condition is a proliferative disorder and, in certain embodiments, the composition further includes an anti-cancer agent. Exemplary proliferative diseases include, but are not limited to, tumors, begnin neoplasms, pre-malignant neoplasms (carcinoma in situ), and malignanat neoplasms (cancers).

Exemplary cancers include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypercosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a combination of one or more leukemia/lymphoma as described above; and multiple myeloma (MM)), heavy chain discase (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), leiomyosarcoma (LMS), mastocytosis (e.g., systemic mastocytosis), myclodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myclocytic leukemia (CML), chronic neutrophilic leukemia (CNL), hypercosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

Anti-cancer agents encompass biotherapeutic anti-cancer agents as well as chemotherapeutic agents.

Exemplary biotherapeutic anti-cancer agents include, but are not limited to, interferons, cytokines (e.g., tumor necrosis factor, interferon α, interferon α), vaccines, hematopoictic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), cpipodophyllins (e.g. ctoposide, ctoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, cdatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabinc), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca2+ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandctanib (ZACTIMA®, ZD6474), vatalanib (PTK787, PTK/ZK), trastuzumab (HERCEPTIN®), bevacizumab (AVASTIN®), rituximab (RITUXAN®), cctuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoc) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melaminc.

In certain embodiments, the condition is an inflammatory disorder and, in certain embodiments, the composition further includes an anti-inflammatory agent. The term “inflammatory disorder” refers to those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

Exemplary inflammatory disorders include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, type 2 diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenia purpura, interstitial cystitis (painful bladder syndrome), gastrointestinal disorder (e.g., selected from peptic ulcers, regional enteritis, diverticulitis, gastrointestinal bleeding, cosinophilic gastrointestinal disorders (e.g., cosinophilic esophagitis, cosinophilic gastritis, cosinophilic gastroenteritis, cosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious ancacmia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis.

In certain embodiments, the inflammatory disorder is inflammation associated with a proliferative disorder, e.g., inflammation associated with cancer.

In certain embodiments, the condition is an autoimmune disorder and, in certain embodiments, the composition further includes an immunomodulatory agent. Exemplary autoimmune disorders include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, cosinophilic disease, gastrointestinal disorder (e.g., selected from peptic ulcers, regional enteritis, diverticulitis, gastrointestinal bleeding, eosinophilic gastrointestinal disorders (e.g., cosinophilic esophagitis, cosinophilic gastritis, cosinophilic gastroenteritis, cosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); cosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

In certain embodiments, the condition is a painful condition, and, in certain embodiments, the composition further includes an analgesic agent. A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawl symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise combinations of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

In certain embodiments, the painful condition is inflammatory pain. In certain embodiments, the painful condition (e.g., inflammatory pain) is associated with an inflammatory disorder and/or an autoimmune disorder.

In certain embodiments, the condition is a liver disease and, in certain embodiments, the composition further includes an agent useful in treating liver disease. Exemplary liver diseases include, but are not limited to, drug-induced liver injury (e.g., acetaminophen-induced liver injury), hepatitis (e.g., chronic hepatitis, viral hepatitis, alcohol-induced hepatitis, autoimmune hepatitis, steatohepatitis), non-alcoholic fatty liver disease, alcohol-induced liver disease (e.g., alcoholic fatty liver, alcoholic hepatitis, alcohol-related cirrhosis), hypercholesterolemia (e.g., severe hypercholesterolemia), transthyretin-related hereditary amyloidosis, liver cirrhosis, liver cancer, primary biliary cirrhosis, cholestatis, cystic disease of the liver, and primary sclerosing cholangitis. In certain embodiments the liver disease is associated with inflammation.

In certain embodiments, the condition is a familial amyloid neuropathy and, in certain embodiments, the composition further includes an agent useful in a familial amyloid neuropathy.

Compositions comprising a polymer of the invention may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. Compositions are preferably formulated in dosage unit form for case of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

### Definitions

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a corrosion inhibitor” is a reference to one or more of such corrosion inhibitors and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. In some embodiments, “about X” (where X is a numerical value) refers to ±10% of the recited value, inclusive. For example, the phrase “about 8” can refer to a value of 7.2 to 8.8, inclusive. This value may include “exactly 8”. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds and polymers described herein can comprise one or more asymmetric centers, and thus can exist in various stereoisomeric forms, e.g., enantiomers and/or diastereomers. For example, the polymers described herein can be in the form of an individual enantiomer, diastereomer or geometric isomer, or can be in the form of a combination of stereoisomers, including racemic combinations and combinations enriched in one or more stereoisomer. Isomers can be isolated from combinations by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stercochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses polymers as individual isomers substantially free of other isomers, and alternatively, as combinations of various isomers.

Unless otherwise stated, structures depicted herein are also meant to include polymers that differ only in the presence of one or more isotopically enriched atoms. For example, polymers having the present structures except for the replacement of hydrogen by deuterium or tritium, replacement of ¹⁹F with ¹⁸F, or the replacement of a carbon by a ¹³C-or ¹⁴C-enriched carbon are within the scope of the disclosure. Such polymers are useful, for example, as analytical tools or probes in biological assays.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the polymers of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-OH-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline carth metal, ammonium, and N⁺(C₁₋₄ alkyl)₄— salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In certain embodiments, the polymer of Formula (I) is a salt. In certain particular embodiments, the polymer of Formula (I) is a pharmaceutically acceptable salt.

In certain embodiments, one or more radicals of Formulac (A), (B), (C), and (D) are salts. In certain particular embodiments, one or more radicals of Formulae (A), (B), (C), and (D) are pharmaceutically acceptable salts.

The terms “composition” and “formulation” are used interchangeably.

As used herein, the term “polyplex” refers to a complex comprising a polymer of the invention and one or more agents. In certain embodiments, a polyplex takes the form of a particle, such as a nanoparticle.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, cows, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. A “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant produces fruit. In some embodiments, the plant is a tree or shrub.

The term “administer,” “administering,” or “administration” refers to implanting, absorbing, ingesting, injecting, inhaling, or otherwise introducing a polymer described herein, or a composition thereof, in or on a subject.

The terms “treatment,” “treat,” and “treating” refer to reversing, alleviating, delaying the onset of, or inhibiting the progress of a disease (e.g., a bacterial infection) described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent,” “preventing,” or “prevention” refers to a prophylactic treatment of a subject who is not and was not with a disease (e.g., a bacterial infection) but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “condition,” “disease,” and “disorder” are used interchangeably.

In general, the “effective amount” of an active ingredient refers to an amount sufficient to elicit the desired biological response. As will be appreciated by those of ordinary skill in this art, the effective amount of a polymer of the invention may vary depending on such factors as the desired biological endpoint, the pharmacokinetics of the active ingredient, the disease being treated, the mode of administration, and the age, health, and condition of the subject. An effective amount encompasses therapeutic and prophylactic treatment.

As used herein, and unless otherwise specified, a “therapeutically effective amount” of an active ingredient is an amount sufficient to provide a therapeutic benefit in the treatment of a disease, disorder or condition, or to delay or minimize one or more symptoms associated with the disease, disorder or condition. A therapeutically effective amount of an active ingredient means an amount of the active ingredient, alone or in combination with other agents or therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of an active ingredient is an amount sufficient to prevent a disease, disorder or condition, or one or more symptoms associated with the disease, disorder or condition, or prevent its recurrence. A prophylactically effective amount of an active ingredient means an amount of the active ingredient, alone or in combination with other agents or therapies, which provides a prophylactic benefit in the prevention of the disease, disorder or condition. The term “prophylactically effective amount” can encompass an amount that improves overall prophylaxis or enhances the prophylactic efficacy of another prophylactic agent.

The present invention is further defined in the following Examples. It should be understood that these examples, while indicating preferred embodiments, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and condition.

### EXAMPLES

### General Synthetic Procedures

A round bottomed flask equipped with a magnetic stir bar was charged with bis-electrophile (monomer A, 1 equiv.), linker nucleophile(s) (monomer(s) B, 0-0.7 total equiv.), branching nucleophile(s) (monomer(s) C, 0-0.8 total equiv.), and solvent (1 mL per 50 mg-1 g of material) under inert gas. The reaction mixture was stirred for 4 hr at 23-60° C. and then stirred for 48 hr at 60-120° C. The mixture was then cooled to room temperature and end cap amine(s) were added (monomer D, 0-2 equiv.) and further stirred for 24 hr. The polymer was then purified by dropwise precipitation into cold diethyl ether acidified with glacial acetic acid, and centrifuged at 1200 G for 3 min. The supernatant was discarded and this process was repeated until the supernatant was transparent and then the polymer was dried under vacuum. Polymers were stored at −20° C.

### Example 1—Polymer 76 (Proposed Exemplary Monomeric Unit)

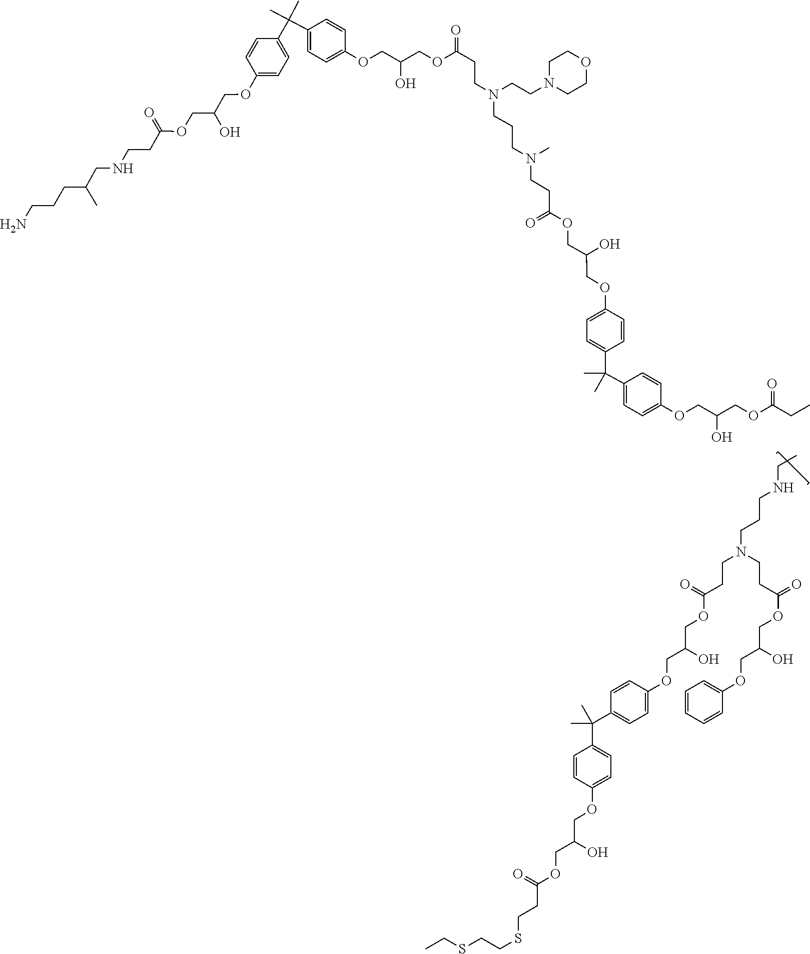

To synthesize polymer 76, acrylate: dithiol: backbone amine: trifunctional amine monomers were reacted modifying the ratio at 1:0.1:0.4:0.20. Monomers were stirred in anhydrous dimethylformamide at a concentration of 150 mg mL⁻¹ at 40° C. for 4 h then 90° C. for 48 h. The mixtures were cooled to 30° C. and the end cap amine was added at 1.5 molar equivalent relative to the acrylate and the reaction was stirred for a further 24 h. The polymers were purified by dropwise precipitation into cold anhydrous diethyl ether with 0.1% glacial acetic acid, and centrifuged at 1250 G for 2 min. The supernatant was discarded and the polymer washed twice in fresh diethyl ether and dried under vacuum for 48 h. The precipitation was repeated until the supernatant looks transparent (the precipitate looks white-yellowish and the supernatant white). Polymers were stored at −20° C.

Specifically, polymer 76 was prepared in a 250 mL round-bottomed flask (RB) equipped with a magnetic stir bar and rubber septum was charged with bisphenol a diglycidyl ether diacrylate (9.08 g, 18.7 mmol, 1 equivalent), ethanedithiol (176 mg, 1.87 mmol, 0.1 equiv.) (d=1.12, V=176 mg/1.12=157 uL) 2-morpholinoethan-1-amine (974 mg (982 uL), 7.5 mmol, 0.4 equivalent), N1-methylpropane-1,3-diamine (330 mg (391 uL), 3.75 mmol, 0.2 equiv.) and DMF (70 mL) under argon. The mixture was stirred at 40° C. for 4 hours and then stirred at 90° C. for 48 hours. The mixture was then cooled to room temperature and 2-methylpentane-1,5-diamine (3.25 g (3.776 mL), 28 mmol, 1.5 equiv.) was added by syringe and the reaction mixture was stirred for another 24 hours.

The polymer was then purified by dropwise addition into cold anhydrous diethyl ether (280 mL) containing 0.1% glacial acetic acid in a 500 mL RB under vigorous stirring. Once the dropwise addition is completed, the mixture was allowed to settle for 10 minutes. The white-colored supernatant was discarded and the sedimented sticky polymer was washed further with diethyl ether containing 0.1% glacial acetic acid under stirring for 15 minutes. This washing process was repeated until the ether layer was transparent. The sticky polymers were washed two more times with fresh diethyl ether (pure) (free from glacial acetic acid) and socked in pure diethyl ether for 12 h under stirring at room temperature (RT) to completely remove the ether soluble impurities. Ether discarded and dried under vacuum for 48 h. A yellowish-white powder was obtained. Polymers were stored at −20° C.

### Example 2—Polymer 38 (Proposed Exemplary Monomeric Unit)

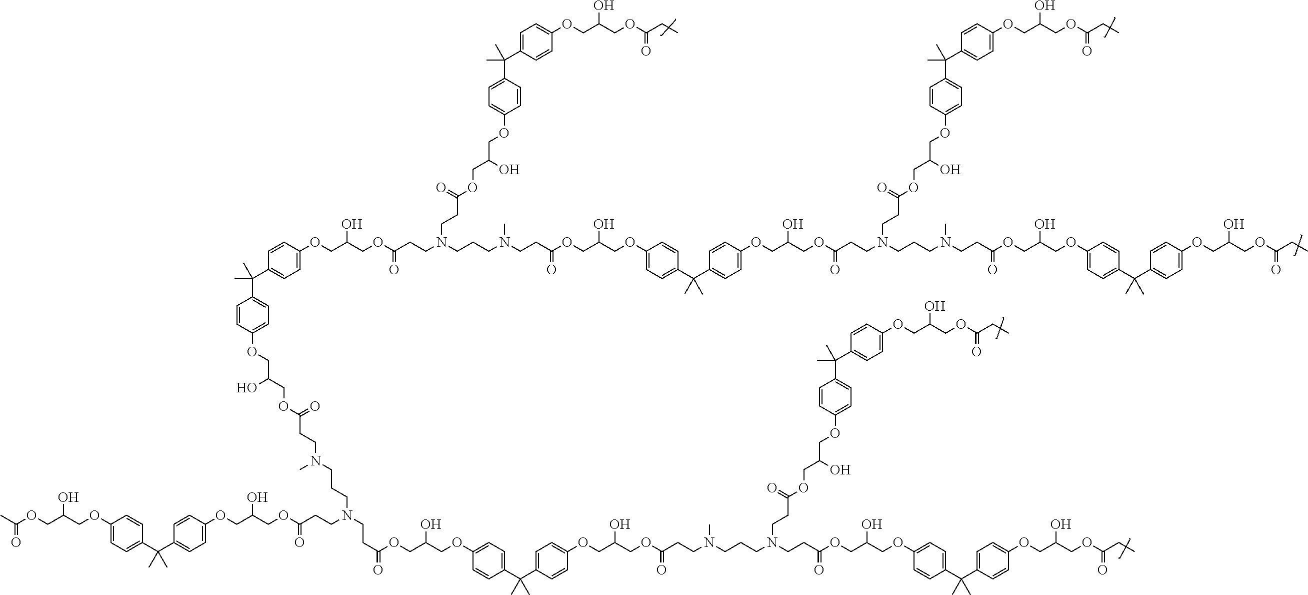

To synthesize polymer 38, acrylate: trifunctional amine monomers were reacted modifying the ratio at 1:0.20. Monomers were stirred in anhydrous dimethylformamide at a concentration of 150 mg mL⁻¹ at 40° C. for 4 h then 90° C. for 48 h. The mixtures were cooled to 30° C. and the end cap amine was added at 1.5 molar equivalent relative to the acrylate and stirred for a further 24 h. The polymers were purified by dropwise precipitation into cold anhydrous diethyl ether with 0.1% glacial acetic acid, and centrifuged at 1250 G for 2 min. The supernatant was discarded and the polymer washed twice in fresh diethyl ether and dried under vacuum for 48 h. The precipitation was repeated until the supernatant looks transparent (the precipitate looks white-yellowish and the supernatant white). Polymers were stored at −20° C.

### Example 3—Polymer 94 (Proposed Exemplary Monomeric Unit)

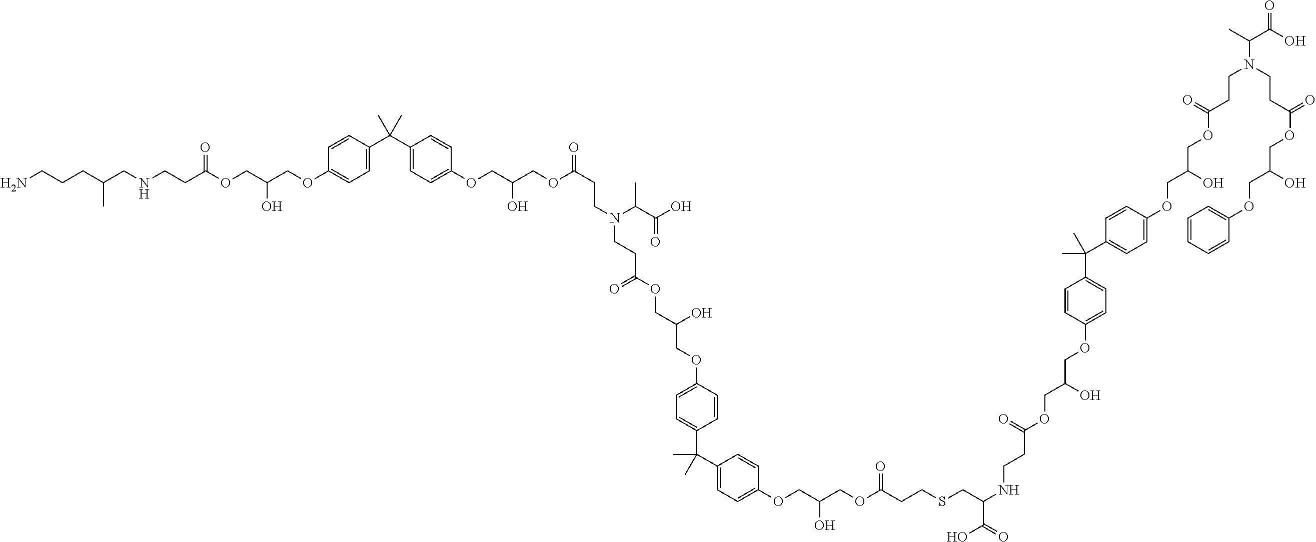 [SMILES: CC(CCCN)CNCCC(=O)OCC(O)COc1ccc(C(C)(C)c2ccc(OCC(O)COC(=O)CCN(CCC(=O)OCC(O)COc3ccc(C(C)(C)c4ccc(OCC(O)COC(=O)CCSCC(NCCC(=O)OCC(O)COc5ccc(C(C)(C)c6ccc(OCC(O)COC(=O)CCN(CCC(=O)OCC(O)COc7ccccc7)C(C)C(=O)O)cc6)cc5)C(=O)O)cc4)cc3)C(C)C(=O)O)cc2)cc1]

To synthesize polymer 94, acrylate: trifunctional amine monomers were reacted modifying the ratio at 1:0.50:0.20. Monomers were stirred in anhydrous dimethylformamide at a concentration of 150 mg mL⁻¹ at 40° C. for 4 h then 90° C. for 48 h. The mixtures were cooled to 30° C. and the end cap amine was added at 1.5 molar equivalent relative to the acrylate and stirred for a further 24 h. The polymers were purified by dropwise precipitation into cold anhydrous diethyl ether with 0.1% glacial acetic acid, and centrifuged at 1250 G for 2 min. The supernatant was discarded and the polymer washed twice in fresh diethyl ether and dried under vacuum for 48 h. The precipitation was repeated until the supernatant looks transparent (the precipitate looks white-yellowish and the supernatant white). Polymers were stored at −20° C.

### Example 4—Polymer 116 (Proposed Exemplary Monomeric Unit)

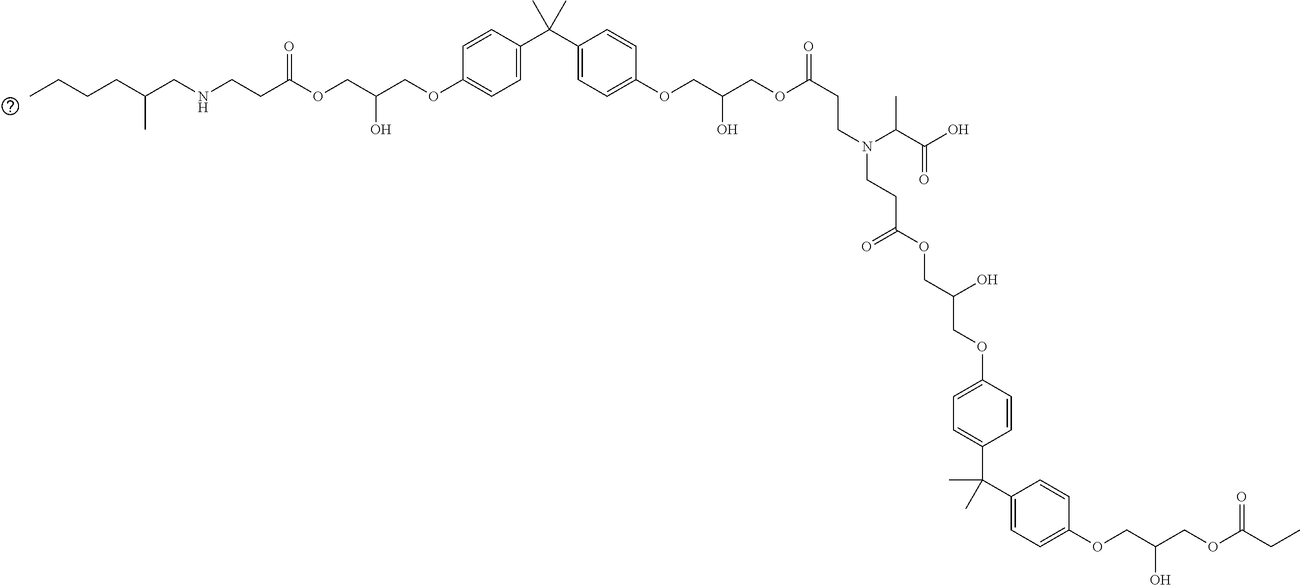 [SMILES: CCCCC(C)CNCCC(=O)OCC(O)COc1ccc(C(C)(C)c2ccc(OCC(O)COC(=O)CCN(CCC(=O)OCC(O)COc3ccc(C(C)(C)c4ccc(OCC(O)COC(=O)CC)cc4)cc3)C(C)C(=O)O)cc2)cc1]

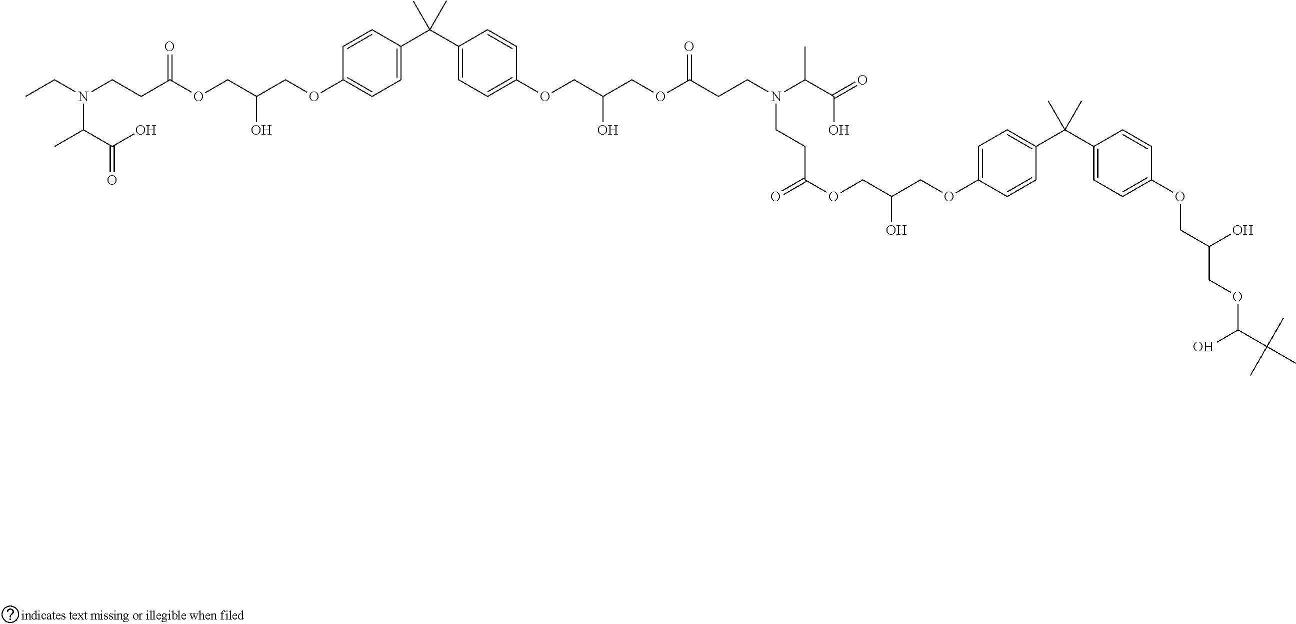 [SMILES: CCN(CCC(=O)OCC(O)COc1ccc(C(C)(C)c2ccc(OCC(O)COC(=O)CCN(CCC(=O)OCC(O)COc3ccc(C(C)(C)c4ccc(OCC(O)COC(O)C(C)(C)C)cc4)cc3)C(C)C(=O)O)cc2)cc1)C(C)C(=O)O]

To synthesize polymer 116, acrylate: trifunctional amine monomers were reacted modifying the ratio at 1:0.50. Monomers were stirred in anhydrous dimethylformamide at a concentration of 150 mg mL−1 at 40° C. for 4 h then 90° C. for 48 h. The mixtures were cooled to 30° C. and the end cap amine was added at 1.5 molar equivalent relative to the acrylate and stirred for a further 24 h. The polymers were purified by dropwise precipitation into cold anhydrous diethyl ether with 0.1% glacial acetic acid, and centrifuged at 1250 G for 2 min. The supernatant was discarded and the polymer washed twice in fresh diethyl ether and dried under vacuum for 48 h. The precipitation was repeated until the supernatant looks transparent (the precipitate looks white-yellowish and the supernatant white). Polymers were stored at −20° C.

### Example 5—Polymer 147 (Proposed Fexemplary Monomeric Unit)

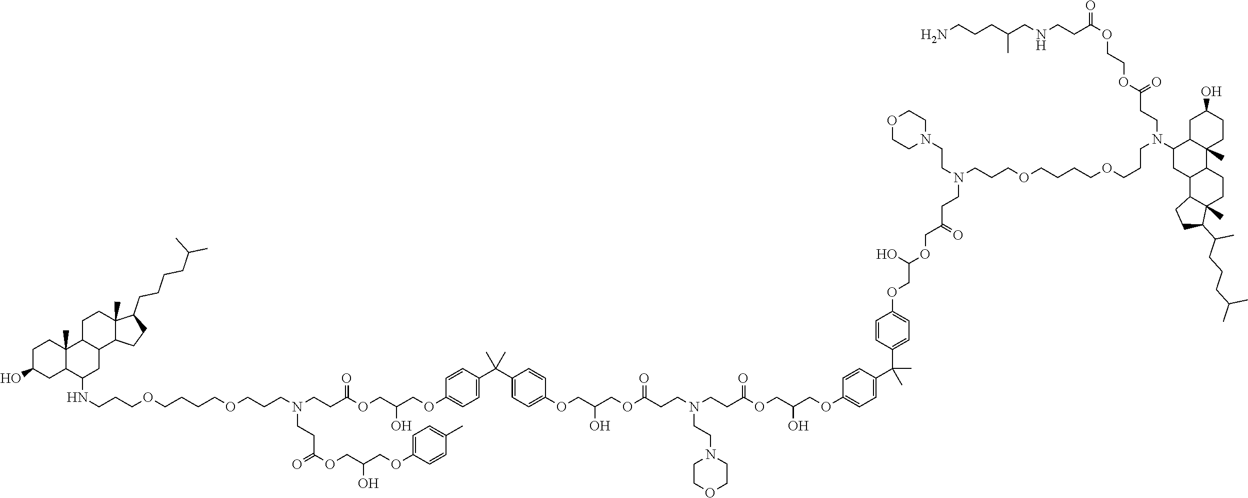

To synthesize polymer 147, acrylate: trifunctional amine monomers were reacted modifying the ratio at 0.8:0.2 for the acrylate mixture and 0.5:0.20 for the amine monomers. Monomers were stirred in anhydrous dimethylformamide at a concentration of 150 mg mL⁻¹ at 40° C. for 4 h then 90° C. for 48 h. The mixtures were cooled to 30° C. and the end cap amine was added at 1.5 molar equivalent relative to the acrylate and stirred for a further 24 h. The polymers were purified by dropwise precipitation into cold anhydrous diethyl ether with 0.1% glacial acetic acid, and centrifuged at 1250 G for 2 min. The supernatant was discarded and the polymer washed twice in fresh diethyl ether and dried under vacuum for 48 h. The precipitation was repeated until the supernatant looks transparent (the precipitate looks white-yellowish and the supernatant white). Polymers were stored at −20° C.

### Example 6—mRNA Delivery to Hamster Lung

The present inventors analyzed the biodistribution of mRNA in hamster lung when delivered with Polymer 76 and Polymer 38 via nebulizer. The biodistribution was determined by microscopy.

aNLuc mRNA was formulated with Polymer 38 and aVHH mRNA was formulated with Polymer 76. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. For delivery of polymer 76 and 38 simultaneously, the formulations were kept separate during the 10 minute incubation period until immediately prior to addition to the nebulizer for delivery to the hamsters.

Polymers were administered via nebulizer at a dosage of about 30 μg per hamster. Lung tissue was excised after 4 hours and analyzed by RNAscope for distribution of the aNLuc mRNA and aVHH mRNA. The results are shown in FIGS. 2 and 3.

FIG. 2 demonstrates that Polymer 76 alone efficiently delivers nebulized mRNA homogenously across the hamster lungs.

FIG. 3 demonstrates that Polymer 76 and Polymer 38 efficiently deliver RNA granules of aNLuc or aVHH mRNA homogenously across the hamster lungs, as well as RNA granules with a combination of aNLuc and aVHH mRNA. These granules were observed primarily in the alveolar space with little airway mRNA signal.

### Example 7—mRNA-Encoded Antibody Delivery to Mouse and Hamster Lung

This study analyzed the expression of mRNA-encoded antibodies in hamster and mouse lung when delivered with Polymer 76, Polymer 38, Polymer 94, Polymer 147 and Polymer 116 via nebulizer. The expression of the mRNA was determined by an encoded nanoluciferase protein that is fused to the C-terminal of the light chain sequence of the IgG antibody. Protein expression via luminescence was then determined by a Perkin Elmer branded in vivo imaging system (IVIS).

aHCA-NLuc mRNA was formulated with each of Polymer 76, Polymer 38, Polymer 94, Polymer 147 and Polymer 116. For comparative purposes, data from Anderson (hDD90-118) is included. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. With respect to mice, formulations were delivered via nebulizer at dosages of 25 μg, 50 μg and 100 μg of aHCA-NLuc mRNA per animal. With respect to hamsters, formulations were delivered via nebulizer at dosages of 30 μg, 60 μg and 125 μg of aHCA-NLuc mRNA per animal.

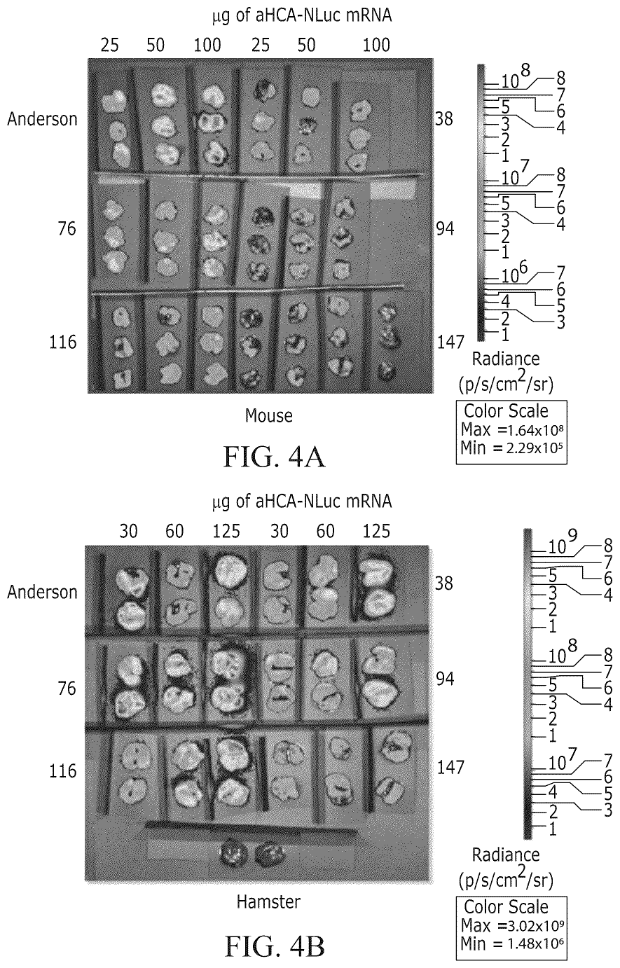

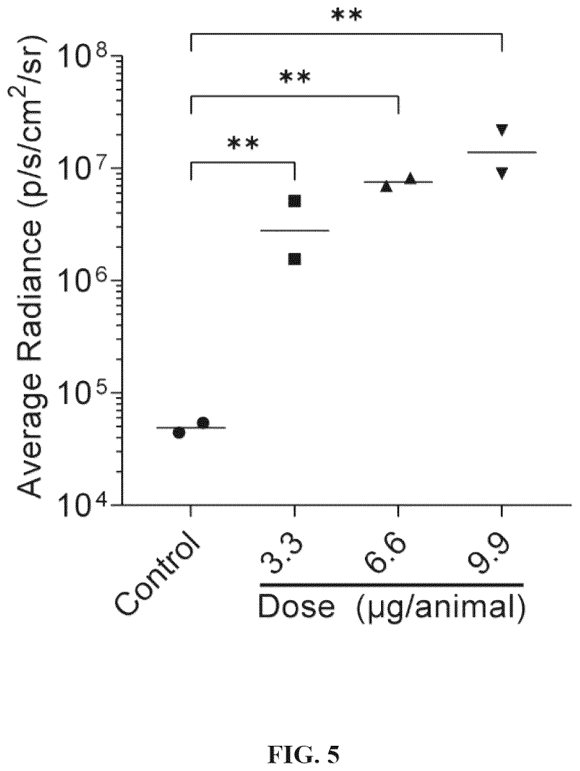

The results are shown in FIG. 4, which demonstrates that Polymer 76, Polymer 38, Polymer 94, Polymer 147 and Polymer 116 efficiently delivers antibodies to the lungs of mice (FIG. 4A) and hamsters (FIG. 4B). Additional results are shown in FIG. 5 which demonstrate the range of efficiencies in delivering mRNA the hDD90-118 at 3.3 μg/animal, 6.6 μg/animal, or 9.9 μg/animal. Bioluminescence quantified in FIG. 5 was measured 24 hours post treatment with aNLuc mRNA delivered by hDD90-118. These results depicted in FIG. 5 demonstrate the nose-only nebulizer apparatus design minimizes the dead volume (the space between the nebulizer outlet and inhaling region of the animals), allowing for doses as low as 0.165 mg kg⁻¹ (3.3 μg per mouse) of aNLuc mRNA. In addition, the apparatus exhibited minimal animal-to-animal variability in expression, allowing for the use of only two animals per formulation. **p<0.01 (one-way ANOVA with multiple comparisons on log-transformed data). n=3 mice per group. The bars represent the geometric mean.

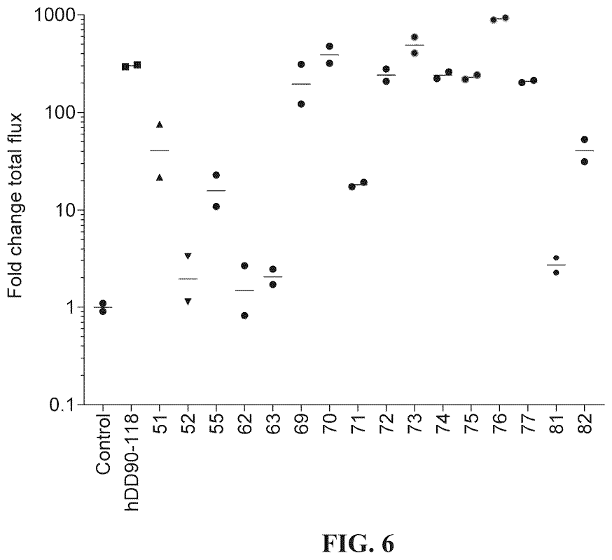

Additional results are shown in FIG. 6 which demonstrate the range of efficiencies in delivering aNLuc mRNA via the indicated polymers at 5 mg/kg dose. Bioluminescence quantified in FIG. 6 was measured 24 hours post treatment with aNLuc mRNA delivered by the indicated polymers. These results depicted in FIG. 6 demonstrate the efficacy of polymer 76 as superiors to the other tested polymers. In addition, the apparatus exhibited minimal animal-to-animal variability in expression, allowing for the use of only two animals per formulation. **p<0.01 (one-way ANOVA with multiple comparisons on log-transformed data). n=3 mice per group. The bars represent the geometric mean.

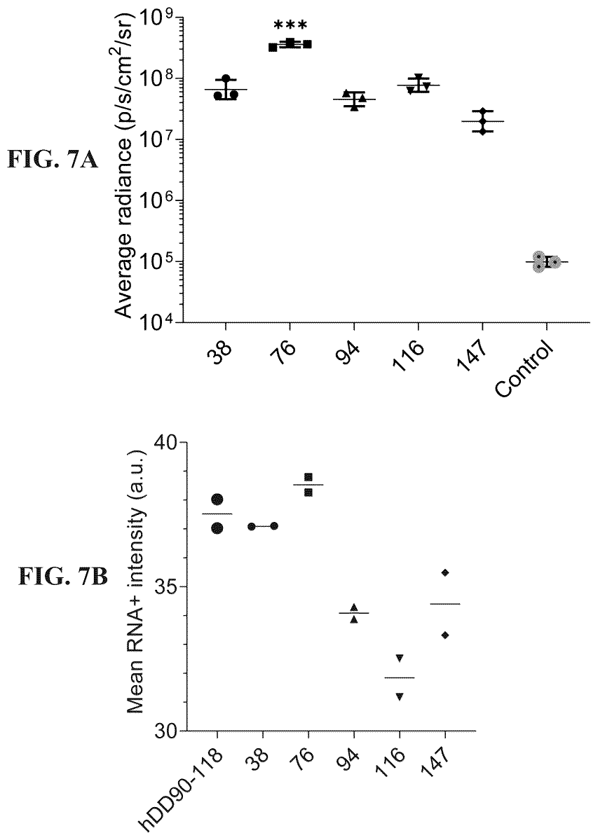

Comparison of the polymer candidates was tested in mouse lungs using a dose of 5 mg/kg. Polymer candidates include Polymer 38, Polymer 76, Polymer 94, Polymer 116, and Polymer 147. Mice were treated with these polymers carrying aNLuc mRNA. Average radiance of bioluminescence in mouse lungs was measured at 24 hours post nebulization (FIG. 7A). Results in FIG. 7A demonstrate that Polymer 76 is more effective at delivering aNLuc mRNA to mouse lungs than Polymer 38, Polymer 94, Polymer 116, and Polymer 147. Mean RNA intensity of aNLuc RNA in RNA granules was measured 4 hours post nebulization (FIG. 7B). Fluorescence in situ hybridization was used to visualize and quantify aNLuc. Segbla (a marker of airway cells), and nuclei stained via 4′,6-diamidino-2-phenylindole (DAPI). Scale bar, 15 mm. n=2 mice per group. Results in FIG. 7B demonstrate that Polymer 76 delivery of aNLuc mRNA yields more aNLuc RNA in RNA granules in mouse lungs than delivery by Polymer 38, Polymer 94, Polymer 116, Polymer 147, or hDD90-118.

### Example 8—Large mRNA Delivery to Hamster Lung

This study analyzed the expression of larger mRNA molecules in hamster lung when delivered with Polymer 76 via nebulizer. The expression of the mRNA was determined by an encoded nanoluciferase protein that is fused to the C-terminal of the sequence of a LbuCas13a molecule. Protein expression via luminescence was then determined by a Perkin Elmer branded in vivo imaging system (IVIS).

Polymer 76 was formulated with 30 μg, 60 μg, and 125 μg of Cas13a-NLuc mRNA and was delivered via nebulizer along with a 1:50 molar ratio of a short, 57 nucleotide, nontargeted control guide RNA. For comparative purposes, data from the Anderson polymer is included. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively.

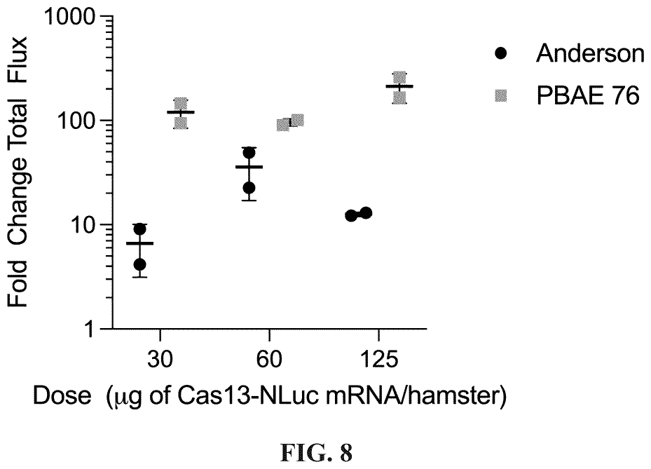

The results are shown in FIG. 8, which demonstrates that Polymer 76 efficiently delivers Cas13a-NLuc mRNA to the lungs of hamsters. Cas13a-NLuc mRNA is a longer chain (e.g., 4600 nt) mRNA and this study demonstrates that Polymer 76 can deliver larger molecules. Delivery with Polymer 76 yielded a substantially flat expression response to dosage increases, whereas hDD90-118 demonstrates a peak at about 60 μg dosage and falls off at the 125 μg dosage.

### Example 9—Cargo Agnostic mRNA Delivery to Mouse Lung

The present inventors analyzed the expression of mRNA molecules in BALB/C and DBA/2 strains of mice lungs when delivered with Polymer 76 via nebulizer. The expression of the mRNA was determined by an encoded nanoluciferase protein that is fused to the C-terminal of the light chain sequence of the HCA IgG antibody, or fused to the C-terminal of either Cas13a protein or dCas9-VPR protein. Protein expression via luminescence was then determined by a Perkin Elmer branded in vivo imaging system (IVIS).

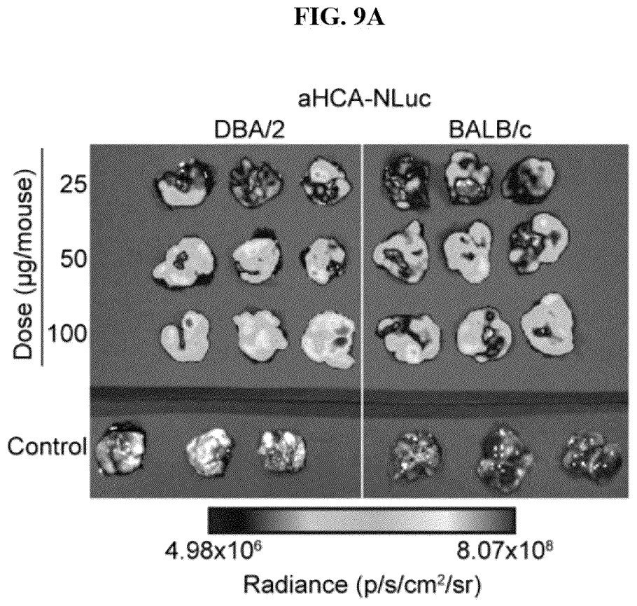

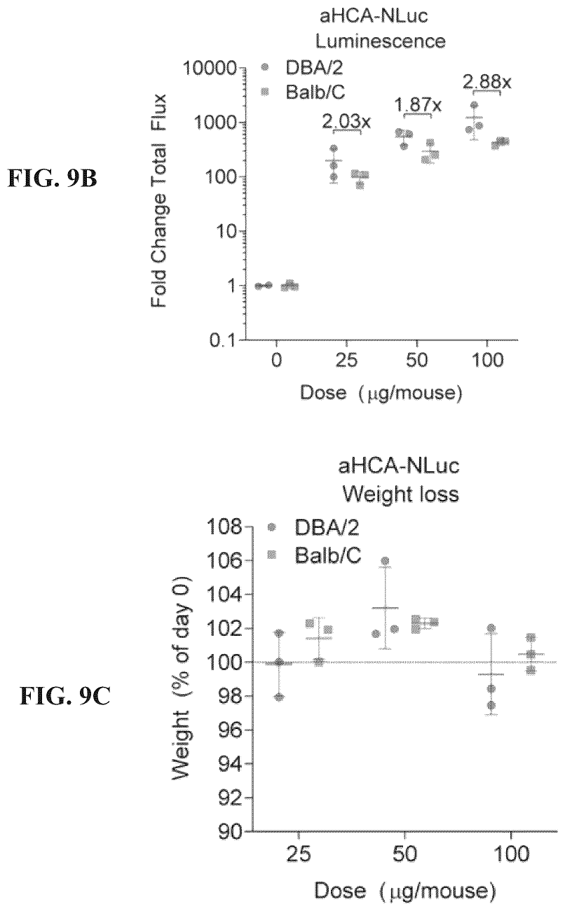

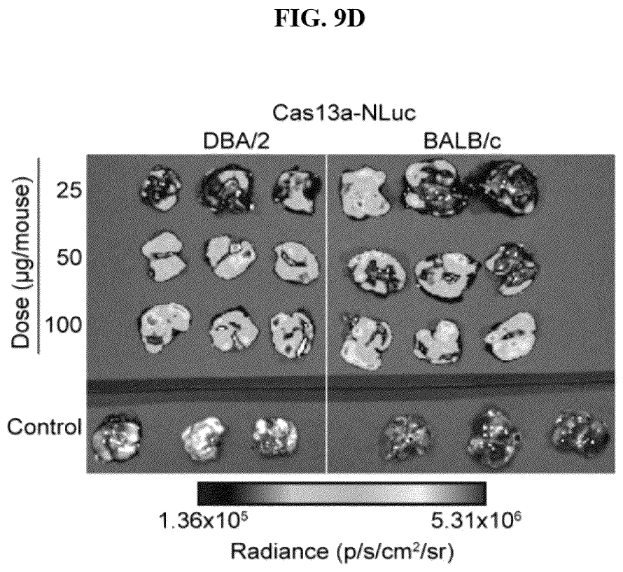

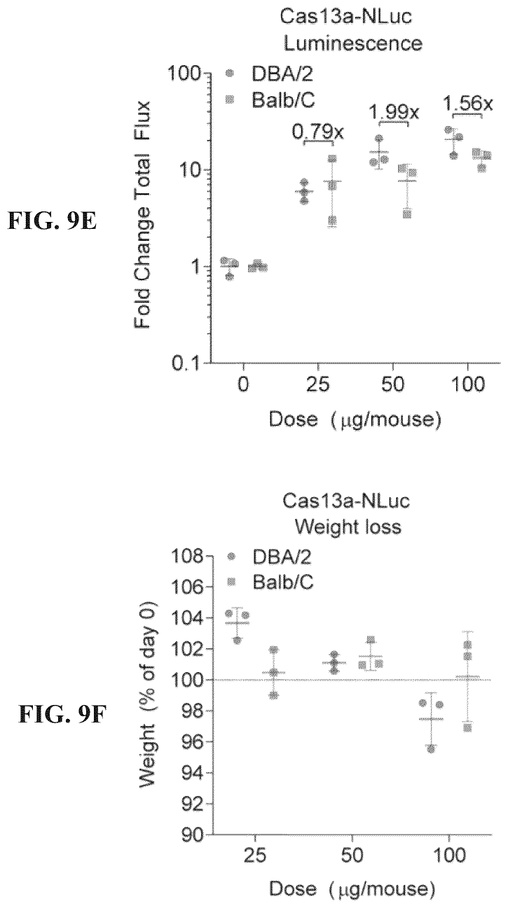

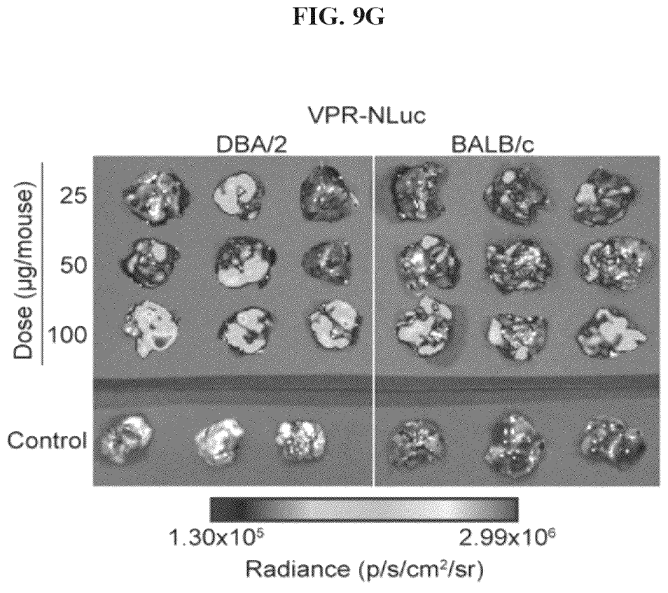

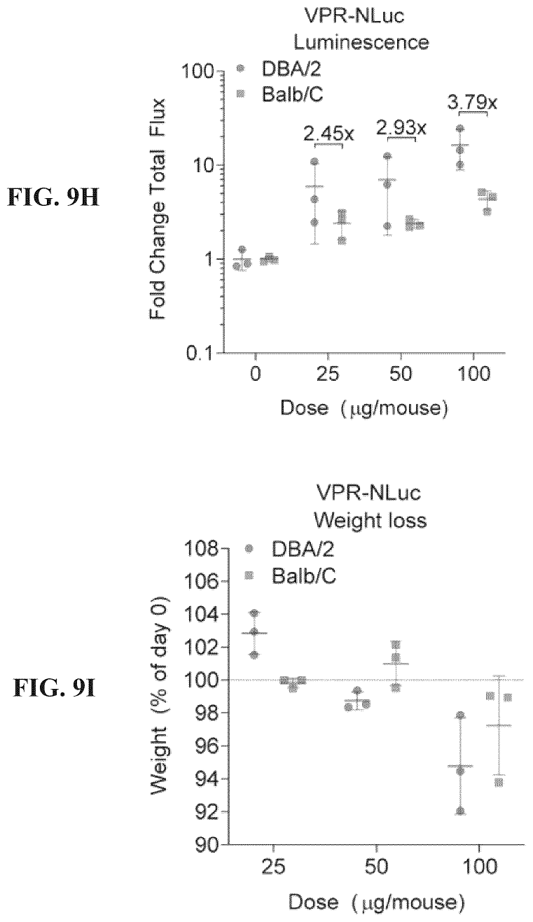

Polymer 76 was formulated with aHCA-NLuc mRNA, or Cas13a-NLuc along with a nontargeting guide RNA, or dCas9-VPR-NLuc along with a nontargeting guide RNA. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. The polymer-formulated mRNA was delivered via nebulizer. The results are shown in FIG. 9, which demonstrates that Polymer 76 efficiently delivers mRNA or various sizes to the lungs of mice, with minimal effects on weight loss which is used as a general health metric. This data also demonstrates that delivery is high to both BALB/C and DBA2 strains of mice.

### Example 10—mRNA Delivery to Ferret Lung

The present inventors analyzed the expression of mRNA molecules in ferret lung when delivered with Polymer 76 via nebulizer. The expression of the mRNA was determined by an encoded nanoluciferase protein that is fused to the C-terminal of the light chain sequence of the HCA IgG antibody. Protein expression via luminescence was then determined by a Perkin Elmer branded in vivo imaging system (IVIS).

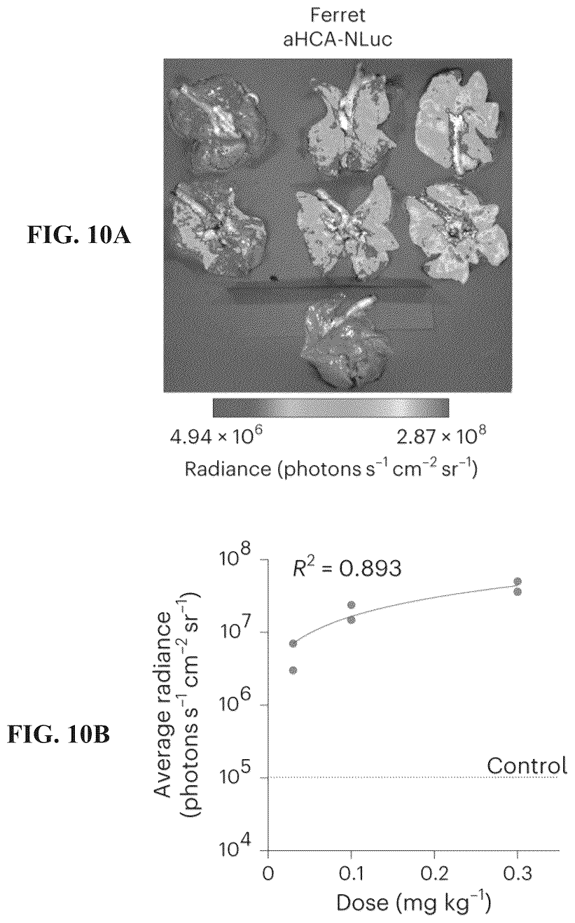

Polymer 76 was formulated with aHCA-NLuc mRNA. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. The polymer-formulated mRNA was delivered via nebulizer. The results are shown in FIG. 10, which demonstrates that Polymer 76 efficiently delivers aHCA-NLuc mRNA to the lungs of ferrets.

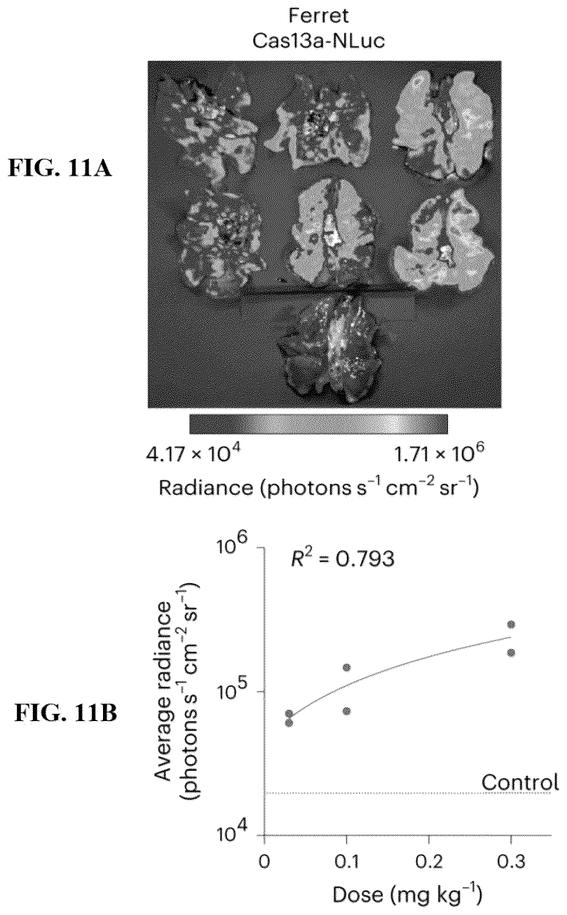

Polymer 76 was formulated with Cas13a-NLuc mRNA. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. The polymer-formulated mRNA was delivered via nebulizer. The results are shown in FIG. 11, which demonstrates that Polymer 76 efficiently delivers Cas13a-NLuc mRNA to the lungs of ferrets.

The results of FIGS. 10 and 11 demonstrates that Polymer 76 efficiently delivers mRNA of various sizes to the lungs of ferrets via nebulizer.

### Example 11—mRNA Delivery Comparison to Mouse, Hamster and Ferret Lung

This study compares the protein expression of mRNA molecules in mice, hamsters and ferrets lungs for aNLuc mRNA when delivered with Polymer 76 via nebulizer. The expression of the mRNA was determined by an encoded nanoluciferase protein. Protein expression via luminescence was then determined by a Perkin Elmer branded in vivo imaging system (IVIS). Polymer 76 was formulated with aNLuc mRNA. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. The mRNA was delivered via nebulizer. The animals were dosed at 0.3 mg/kg and then euthanized and analyzed after 24 hours.

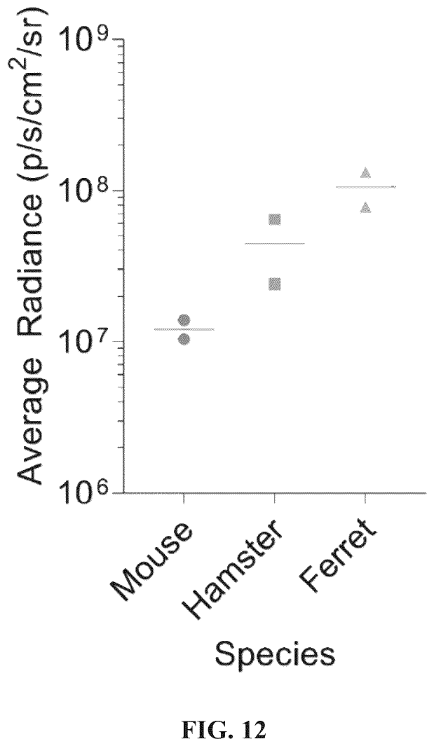

The results are shown in FIG. 12 for average radiance, which demonstrates that Polymer 76 efficiently delivers aNLuc mRNA of each animal. The data of FIG. 12 shows nebulized delivery via Polymer 76 improves as the size of the animals increases.

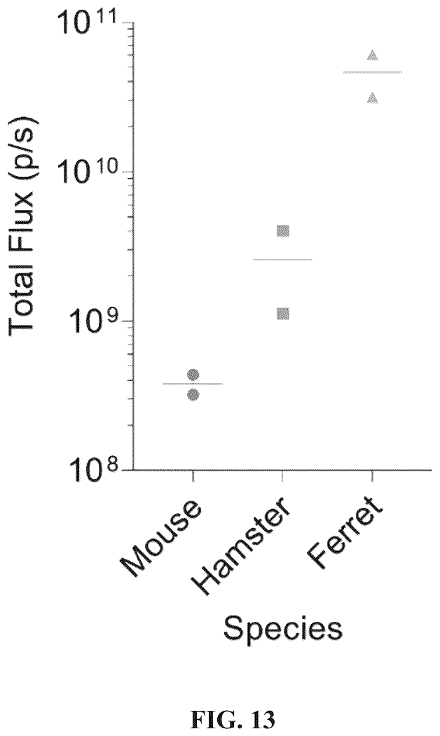

The results are shown in FIG. 13 for total flux, which demonstrates that Polymer 76 efficiently delivers aNLuc mRNA of each animal. The data of FIG. 13 shows nebulized delivery via Polymer 76 improves as the size of the animals increases.

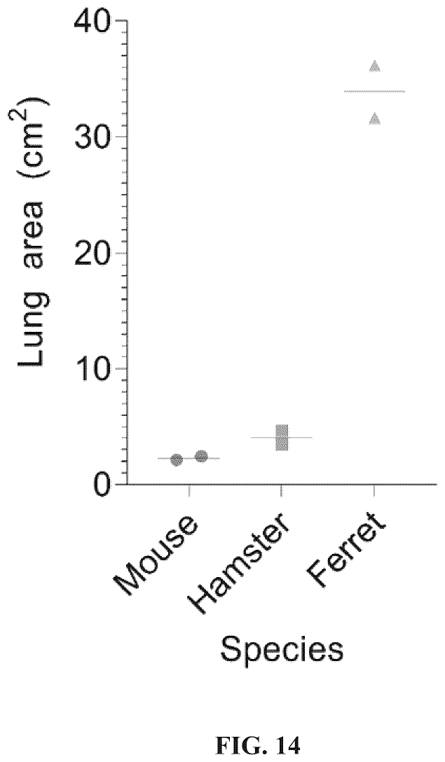

The results are shown in FIG. 14 for total area of the lung, which is larger in the larger animals. The data of FIG. 14 shows nebulized delivery via Polymer 76 improves as the size of the animals increases.

### Example 12—mRNA Delivery to Cow Lung

This study analyzed the protein expression of mRNA molecules in cow lung when delivered with Polymer 76 via nebulizer. The expression of the mRNA was determined by an encoded nanoluciferase protein. Protein expression via luminescence was then determined by a Perkin Elmer branded in vivo imaging system (IVIS).

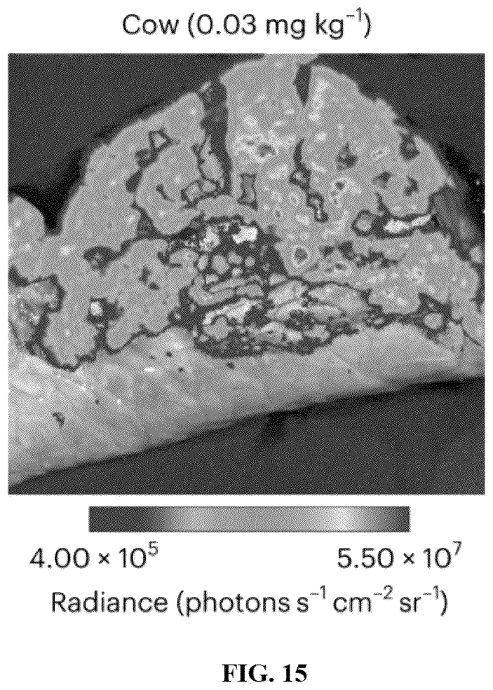

Polymer 76 was formulated with aNLuc mRNA. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. The mRNA was delivered via nebulizer. The cows were dosed at 0.03 mg/kg and then euthanized and analyzed after 24 hours. The results are shown in FIG. 15, which demonstrates that Polymer 76 efficiently delivers aNLuc mRNA to the lungs of cows, even at low doses.

### Example 13—mRNA Delivery to Macaque Lung

This study analyzed the protein expression of mRNA molecules in rhesus macaque lung when delivered with Polymer 76 via nebulizer. The expression of the mRNA was determined by an encoded nanoluciferase protein. Protein expression via luminescence was then determined by a Perkin Elmer branded in vivo imaging system (IVIS).

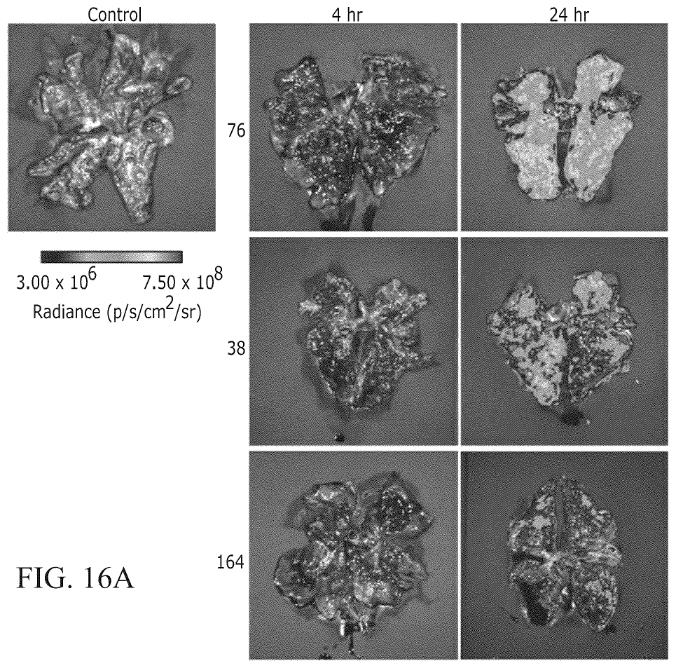

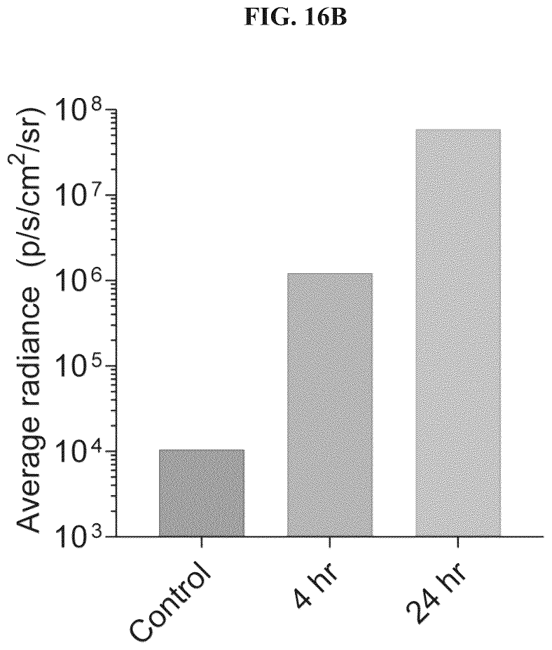

Polymer 76 was formulated with aNLuc mRNA. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. The mRNA was delivered via nebulizer. The macaqueswere dosed at 0.3 mg/kg and then euthanized and analyzed after either 4 or 24 hours. The results are shown in FIG. 16, which demonstrates that Polymers 76 and 38 efficiently delivers aNLuc mRNA to the lungs of rhesus macaques and that expression increases over the first day. In addition, polymer 164 does not deliver to the lungs as well as polymers 76 and 38.

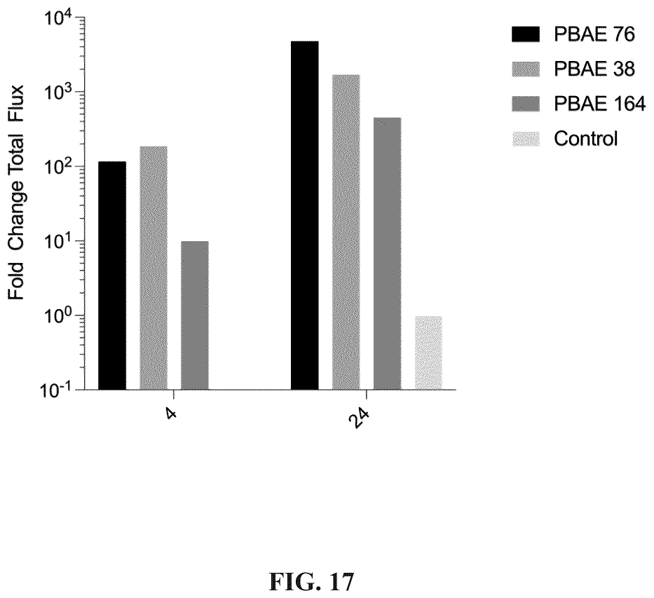

The results are shown in FIG. 17 for Fold Change of Total Flux, which demonstrates that Polymer 76 is effective at delivering mRNA to the lungs of rhesus macaques. The data of FIG. 17 shows nebulized delivery of Polymer 76 is more effective than polymers 38 and 164 at both 4 and 24 hours post transfection.

### Example 14—Localization of mRNA Delivery to Ferret and Macaque Lungs

This study analyzed the distribution of two sizes of mRNA molecules in ferret and macaque lungs when delivered at 0.3 mg/kg with Polymer 76 via nebulizer. The localization of the mRNA and the airway cells was determined by fluorescent in situ hybridization, while nuclei were counterstained with DAPI for cell context.

Polymer 76 was formulated with either aHCA-NLuc or Cas13a-NLuc mRNA for ferrets and aNLuc for macaques. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. The mRNA was delivered via nebulizer. The animals were doscd at 0.3 mg/kg and then euthanized and analyzed after 4 hours. Lungs were extracted, prepared, and sectioned onto slides. Sections were processed using fluorescent in situ hybridization using the ACD RNAscope method for imaging delivered RNA and airway cell marker RNA.

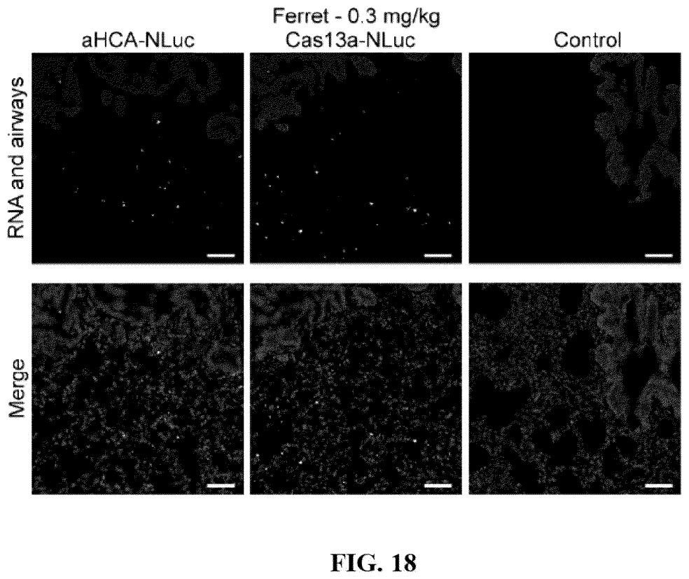

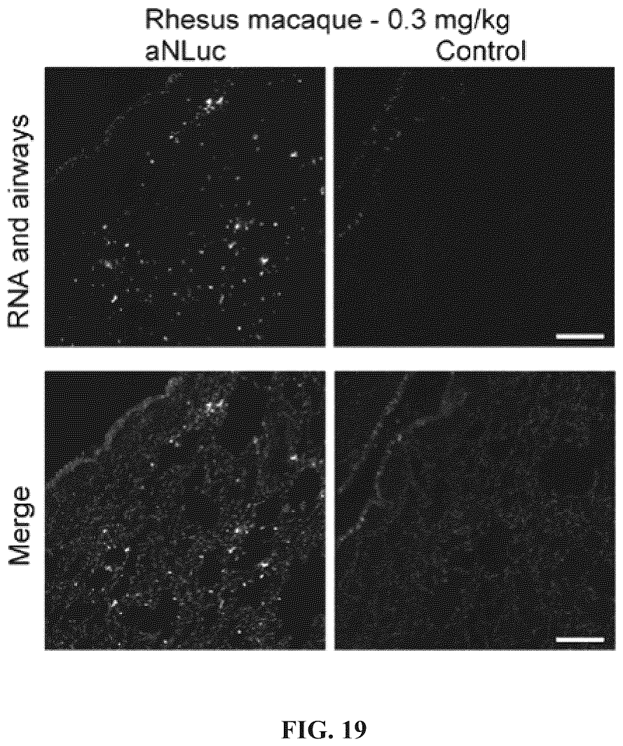

The results shown in FIG. 18 demonstrate the alveolar localization of mRNA delivery of all sizes of cargo in the ferret model.

The results shown in FIG. 19 demonstrate the alveolar localization of mRNA delivery in the rhesus macaque model.

### Example 15—Histology of mRNA Delivery to Lung

This study analyzed the effect on lung pathology of mRNA molecules in ferret and rhesus macaque lung when delivered at 0.3 mg/kg with Polymer 76 via nebulizer.

Polymer 76 was formulated with either aHCA-NLuc or Cas13a-NLuc mRNA for ferrets and aNLuc for macaques and mice. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. The mRNA was delivered via nebulizer. The animals were dosed at 0.3 mg/kg and then euthanized and analyzed after 4 hours. Lungs were extracted, prepared, and sectioned onto slides. Histology was performed using H&E staining on tissue sections. In ferrets, aHCA-NLuc and Cas13a-NLuc mRNA was used, while in macaques and mice, aNLuc mRNA was used. Control animals were treated with buffer only.

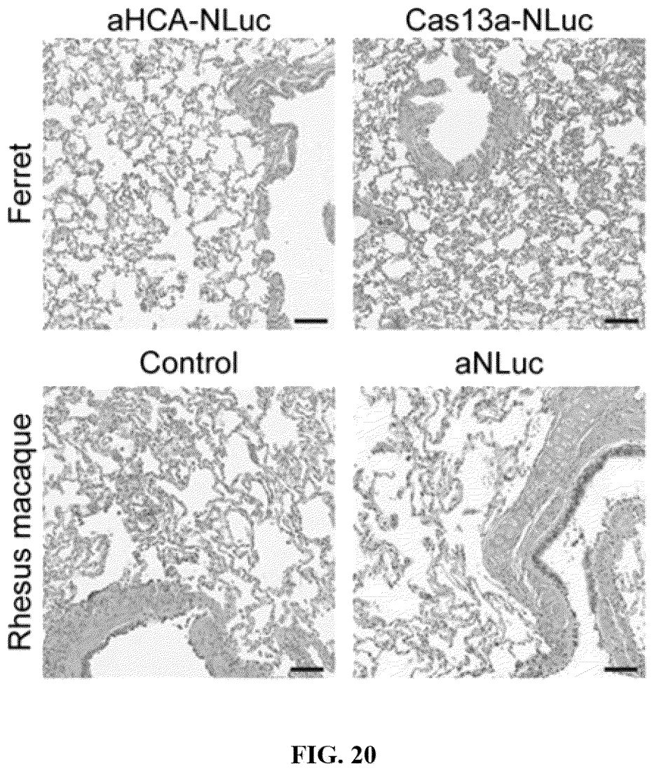

A toxicology study in Rhesus macaques was performed with a single 0.3 mg/kg dose (FIG. 20). The results shown in FIG. 20 demonstrate the minimal lung pathology induced in the lung in the ferret and macaque models after RNA delivery. The analysis of serum before and after dosing in the two macaques (animal IDs A2L084 and A10R078) revealed minimal increases in serum cytokine levels (Table 5). Blood chemistry (Table 6) and hematology (Table 7) analyses revealed only minor changes in most metrics, with all but one remaining within normal levels for macaques. Taken together, these data support the nebulized use of P76 for high mRNA delivery with minimal toxicity in future preclinical applications.

TABLE 5
NHP serum cytokine concentrations in Rhesus macaques 24 hours
post treatment with a 0.3 mg/kg dose of aHCA-NLuc mRNA.

| | A2L084 | | A10R078 | | |
|---|---|---|---|---|---|
| Analyte | Predose | 4 hours | Predose | 24 hours | Units |
| G CSF | 0 | 12.4 | 0 | 0 | pg/mL |
| GMCSF | 0 | 0 | 0 | 0 | pg/mL |
| IFNg | 0 | 0 | 0 | 0 | pg/mL |
| IL-1b | 0 | 0 | 0 | 0 | pg/mL |
| IL-1Ra | 7.97 | 13.18 | 24.35 | 18 | pg/mL |
| IL-2 | 9.92 | 7.21 | 11.83 | 8.25 | pg/mL |
| IL-4 | 0 | 0 | 0 | 0 | pg/mL |
| IL-5 | 0 | 0 | 0 | 0 | pg/mL |
| IL-6 | 0 | 0 | 0 | 0 | pg/mL |
| IL-8 | 2221.5 | 3509 | 533.6 | 768.9 | pg/mL |
| IL-10 | 0 | 0 | 0 | 0 | pg/mL |
| IL-12/23 (p40) | 0 | 0 | 0 | 0 | pg/mL |
| IL-13 | 0 | 0 | 0 | 0 | pg/mL |
| IL-15 | 2.3 | 3.37 | 2.29 | 0 | pg/mL |
| IL-17A | 0 | 0 | 0 | 0 | pg/mL |
| IL-18 | 0 | 0 | 0 | 0 | pg/mL |
| MCP-1 | 171 | 229.67 | 149.9 | 112 | pg/mL |
| MIP-1b | 10.4 | 11.01 | 23.3 | 12.67 | pg/mL |
| MIP-1a | 0 | 0 | 0 | 0 | pg/mL |
| sCD40L | 19032 | 3049.2 | 4914.5 | 1310.6 | pg/mL |
| TGFa | 0 | 11.1 | 0 | 0 | pg/mL |
| TNFa | 0 | 0 | 0 | 0 | pg/mL |
| VEGF | 64.76 | 50.4 | 93.7 | 71.63 | pg/mL |

TABLE 6
NHP blood chemistry panel in Rhesus macaques 24 hours
post treatment with a 0.3 mg/kg dose of aHCA-NLuc mRNA.

| | A2L084 | | A10R078 | | | |
|---|---|---|---|---|---|---|
| | | 4 | | 24 | | |
| Metric | Predose | hours | Predose | hours | Normal | Units |
| Glucose | 63 | 61 | 55 | 68 | 51-83 | mg/dL |
| BUN | 15 | 17 | 17 | 17 | 18-28 | mg/dL |
| Creatinine | 0.7 | 0.7 | 1 | 0.9 | 0.39-1.1 | mg/dL |
| Sodium | 150 | 151 | 150 | 150 | 138-152 | mmol/L |
| Potassium | 4.1 | 4.1 | 3.7 | 3.6 | 3.1-4.5 | mmol/L |
| Chloride | 112 | 113 | 110 | 110 | 99-115 | mmol/L |
| Anion Gap | 16 | 18.6 | 19.7 | 17.9 | 18.7-34.4 | mmol/L |
| CO2 | 26.1 | 23.5 | 24 | 25.7 | 10.8-24.8 | mmol/L |
| Phosphorus | 3.3 | 5 | 5.5 | 4.9 | 2.7-7.8 | mg/dL |
| Calcium | 9.2 | 9.6 | 9.3 | 9.2 | 8.2-10.2 | mg/dL |
| Tot. Protein | 7.6 | 7.7 | 7.8 | 7.8 | 5.4-7.8 | g/dL |
| Albumin | 3.2 | 3.3 | 3.7 | 3.7 | 3.0-4.2 | g/dL |
| Globulin | 4.4 | 4.4 | 4.1 | 4.1 | 1.0-4.8 | g/dL |
| Alb/Glob ratio | 0.7 | 0.8 | 0.9 | 0.9 | 0.9-1.8 | |
| Total Bilirubin | 0.2 | 0.2 | 0.2 | 0.2 | 0.0-0.5 | mg/dL |
| LDH | 202 | 158 | 428 | 429 | 153-764 | U/L |
| GGT | 311 | 311 | 58 | 56 | 28-106 | U/L |
| ALK Phos | 235 | 243 | 159 | 153 | 52-581 | U/L |
| ALT | 112 | 122 | 40 | 74 | 22-71 | U/L |
| AST | 26 | 34 | 24 | 79 | 14-48 | U/L |

TABLE 7
NHP hematology panel in Rhesus macaques 24 hours post
treatment with a 0.3 mg/kg dose of aHCA-NLuc mRNA.

| | A2L084 | | A10R078 | | | |
|---|---|---|---|---|---|---|
| | | 4 | | 24 | | |
| Metric | Predose | hours | Predose | hours | Normal | Units |
| WBC | 9 | 6.1 | 11.8 | 9.8 | 5.8-13.8 | ×10³/μL |
| RBC | 6.1 | 6.18 | 5.16 | 5.12 | 4.72-5.92 | ×10⁶/μL |
| Hemoglobin | 12.7 | 12.8 | 12.8 | 12.8 | 11.0-14.0 | g/dL |
| Hematocrit | 39.4 | 39.6 | 39.5 | 39 | 35.9-41.9 | % |
| MCV | 64.6 | 64.1 | 76.5 | 76.3 | 69.9-75.9 | fl |
| MCH | 20.8 | 20.8 | 24.8 | 24.9 | 21.9-24.9 | pg |
| MCHC | 32.2 | 32.4 | 32.5 | 32.7 | 31.2-34.4 | g/dL |
| RDW | 15 | 14.8 | 14.8 | 14.6 | | % |
| Platelet Ct. | 379 | 385 | 409 | 416 | 311-511 | ×10³/μL |
| MPV | 9.6 | 9.1 | 9.3 | 8.6 | | fl |
| Neut | 68.5 | 56.9 | 74.8 | 63.7 | 37.3-77.3 | % |
| Lymph | 20.5 | 30.5 | 20.4 | 28.4 | 18.8-54.8 | % |
| Mono | 5.9 | 7.2 | 3.4 | 6.8 | 0.0-6.9 | % |
| Eos | 4.7 | 5.3 | 1.1 | 1 | 0.0-3.0 | % |
| Baso | 0.4 | 0.1 | 0.3 | 0.1 | 0.0-1.0 | % |
| Neut | 6.2 | 3.5 | 8.8 | 6.2 | | ×10³/μL |
| Lymph | 1.8 | 1.8 | 2.4 | 2.8 | | ×10³/μL |
| Mono | 0.5 | 0.4 | 0.4 | 0.7 | | ×10³/μL |
| Eos | 0.4 | 0.3 | 0.1 | 0.1 | | ×10³/μL |
| Baso | 0 | 0 | 0 | 0 | | ×10³/μL |

The results shown in FIG. 20 and in Tables 5-7 demonstrate the minimal lung pathology induced in the lung in the Rhesus macaque model after RNA delivery.

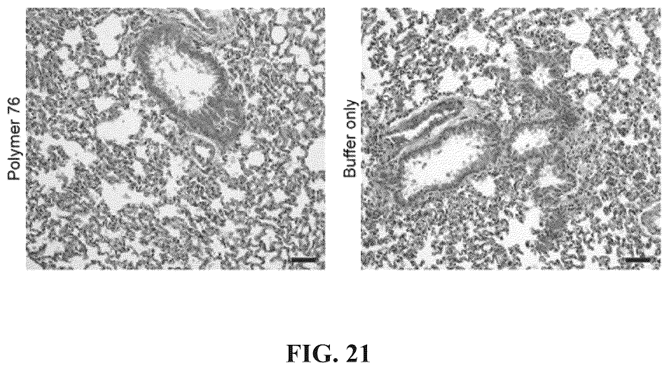

The results shown in FIG. 21 demonstrate the minimal lung pathology induced in the lung in the mouse model after RNA delivery.

### Example 16—Cytokine Gene Modulation from mRNA Delivery to Lung

This study analyzed the effect on mouse lung cytokine RNA levels due to mRNA delivered at 0.3 mg/kg with Polymer 76 via nebulizer. Polymer 76 was formulated with aNLuc mRNA. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. The mRNA was delivered via nebulizer. The animals were dosed at 0.3 mg/kg and then euthanized and analyzed after 24 hours. 24 hours after aNLuc RNA delivery, lungs were harvested, RNA was extracted, and relative cytokine mRNA levels were measured using the NanoString nCounter. Gene expression changes were labeled as significant when p-value is less than 0.05 and fold change up or down was greater than 2.

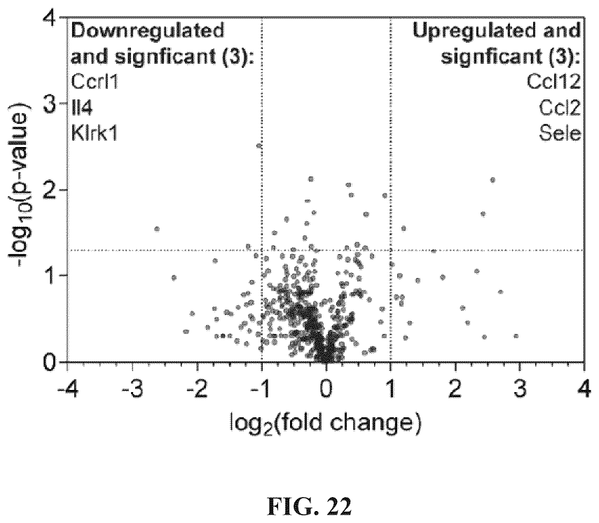

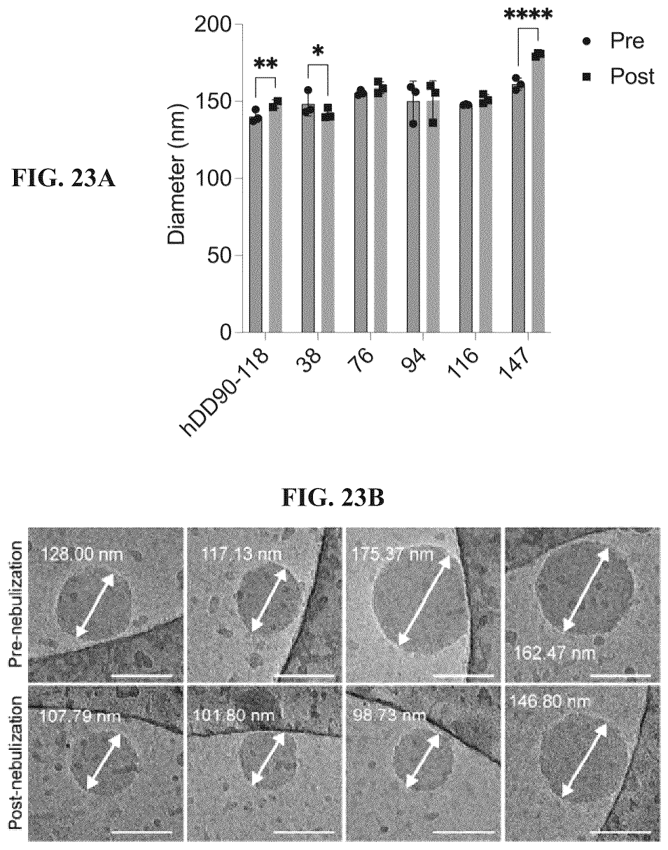

The results shown in FIG. 22 demonstrate the minimal cytokine gene expression changes induced in the lung in the mouse model after delivery of aNLuc mRNA. Additional toxicity of aNLuc mRNA delivered via polymer 76 was analyzed. Mice were treated with aNLuc mRNA delivered via polymer 76 at a dose of 1.25 mg/kg for analysis of lung-tissue-level differential gene expression (FIG. 23). Differential gene expression of 561 inflammatory genes in mouse lungs were measured by NanoString analysis (n=3 mouse lungs per time point) at day 1, 7, and 14 after a single exposure to polymer 76 polyplexes. The horizontal line represents p=0.05 (two-tailed t-test on log-transformed normalized data) and the vertical lines indicate fold changes of ±2. The results shown in FIG. 23 demonstrate no significantly differentially expressed genes at any time point.

### Example 17—Stability of Polyplexes Formed

This study analyzed the effect on polyplex stability due to nebulization when particles are formed with Polymers 38, 76, 94, 116, 147, and the previously reported hDD90-118. Polymers 38, 76, 94, 116, 147 were formulated with aNLuc mRNA for ferrets and aNLuc for macaques. mRNA was first diluted to 1 mg/mL in sodium acetate buffer (pH 5.0). Polymers were dissolved in the same sodium acetate buffer at 50 mg/mL. An equal volume of mRNA and polymer solution was then combined and incubated for 10 minutes; the final concentration of mRNA and polymer is 0.5 mg/mL and 25 mg/mL, respectively. Half of the particles were processed through a nebulizer and collected. Half of the particles were diluted 100 fold in the sodium acetate buffer and diameter was measured by dynamic light scattering (DLS) using a Malvern Zetasizer ZS. The other half of the particles were blotted onto nickel-carbon transmission electron microscopy (TEM) grids, plunge frozen in liquid ethane, and analyzed by cryo-TEM.

Another set of particles was produced with Polymers 38, 76, 94, 116, 147 and measured for surface charge (zeta potential) as another metric of stability. Zeta potential was measured by diluting polyplexes 20-fold in water (pH 5.0), loading the diluted particles into a capillary cuvette, and analyzing the surface charge of the particles using the Malven Zetasizer ZS.

The results shown in FIGS. 23 demonstrate the minimal change in size of polyplexes formed with polymers 38, 76, 94, 116, 147 after nebulization as measured by DLS and cryo-TEM.

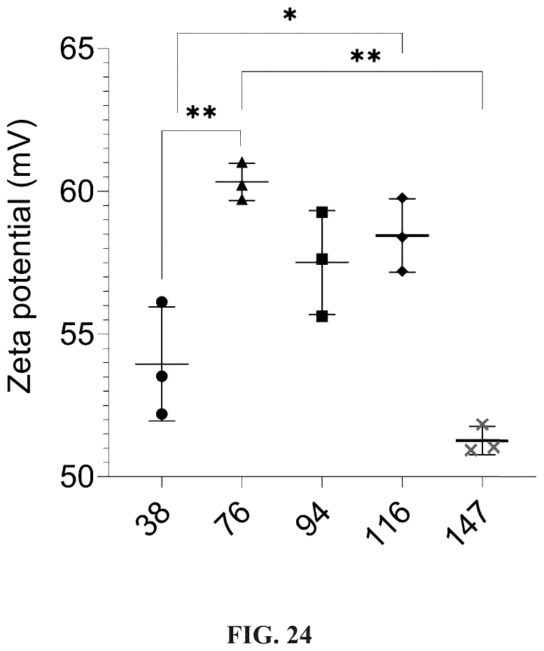

The results shown in FIG. 24 demonstrate the highly positive surface charge of polyplexes formed with polymers 38, 76, 94, 116, 147 as measured by DLS.

### Example 18—Stability of Polyplexes Formed

This study analyzed a potential binding method of Polymer 76 to a representative mRNA strand. All-atom Molecular Dynamics (MD) was applied to investigate the interactions between the polymer and the mRNA. Only a monomer of the polymer being studied was used. From the crystal structure of RNase (7DIC), the nine-base-pair mRNA strand was selected and used to represent the mRNA utilized in experiments

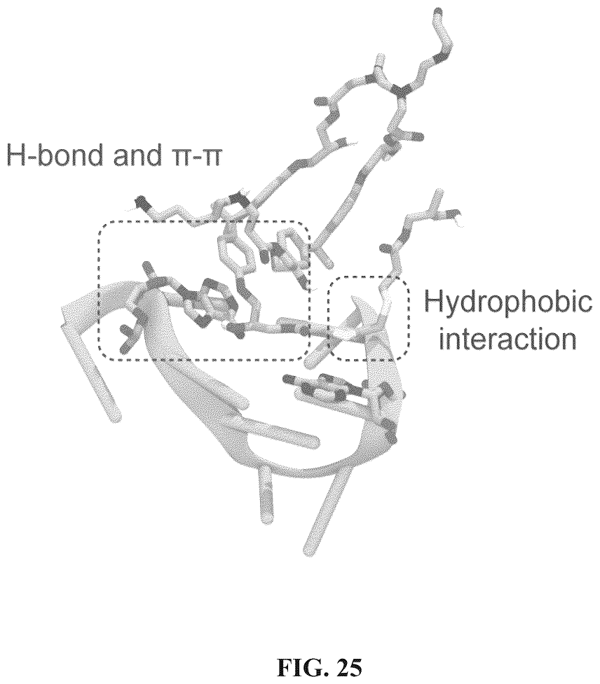

The results shown in FIG. 25 demonstrate hydrogen bonding between the oxygen atoms in the P76 backbone and the bases of the mRNA, as well as π-π interactions between the bisphenol A and the RNA bases.

### Example 19—Analytical Characterization

This study analyzed the incorporation of individual polymer components into the final lead candidate polymers using proton (¹H) nuclear magnetic resonance (NMR). Additionally, this study investigated the incorporation of N-formyl groups from the dimethylformamide (DMF) solvent into the polymers. Specifically, hDD90-118 was prepared in the presence of 10% ¹³C-enriched N,N-dimethylformamide, under otherwise identical conditions to those used elsewhere in this document. The resultant polymer was then analyzed by ¹³C NMR. Given the reaction conditions described (heating in N,N-dimethylformamide solvent at 90° C. for 48 hrs), N-formylation is likely, since DMF is known to be a potential donor of the formyl group.

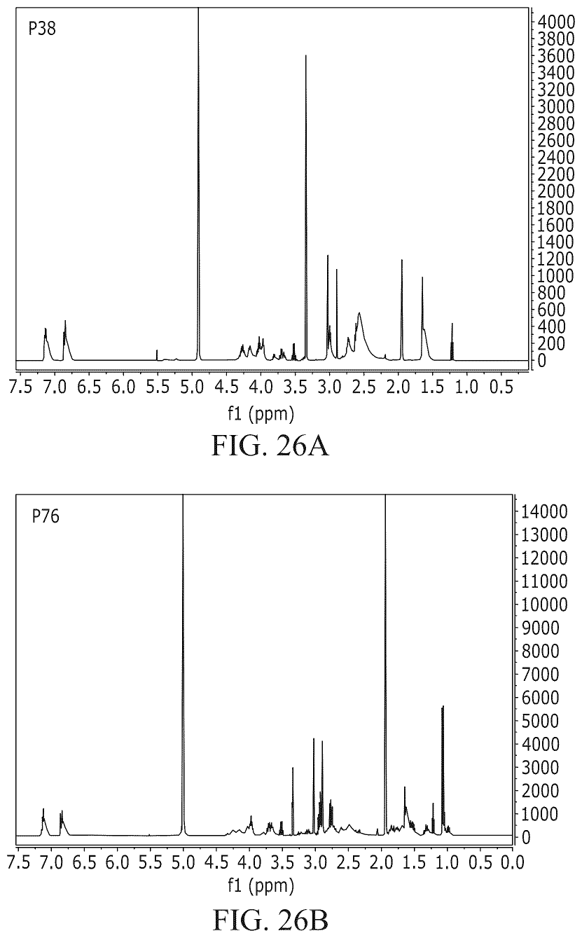

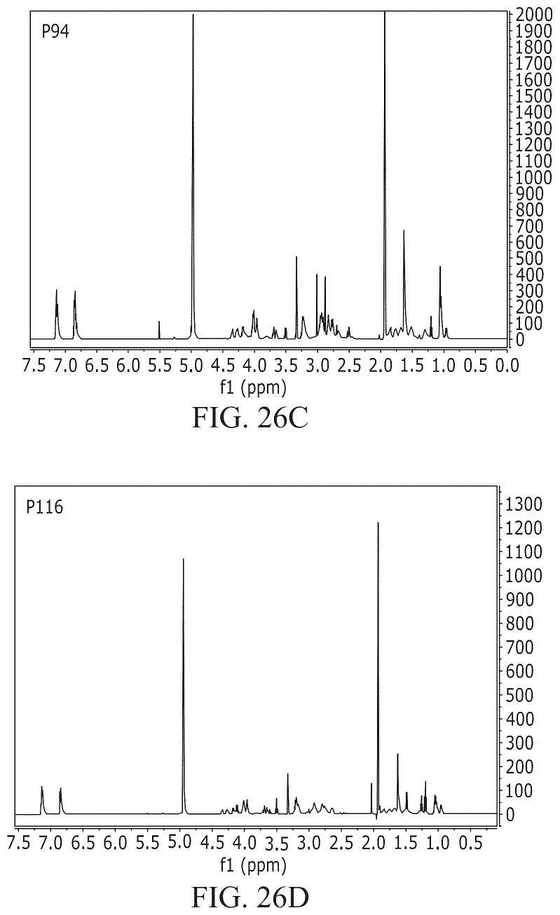

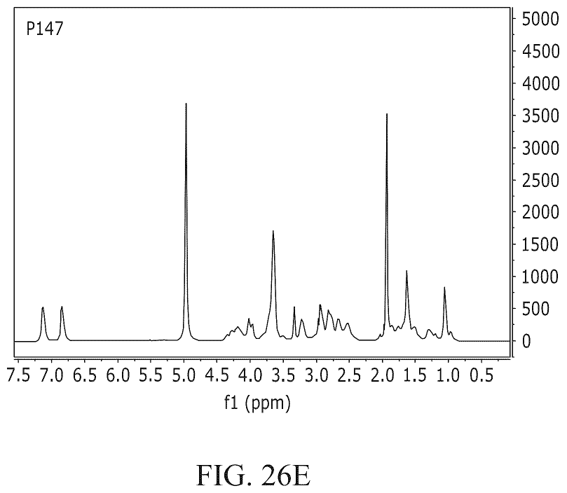

The results shown in FIG. 26 depicts ¹H NMR that demonstrates that for each lead candidate, all reagents have been incorporated in the final product to form a polymer.

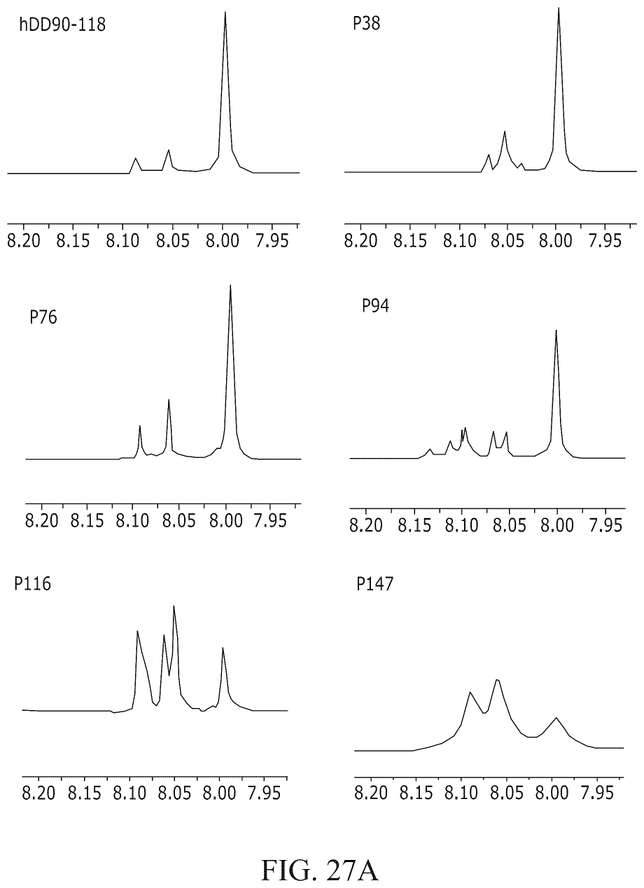

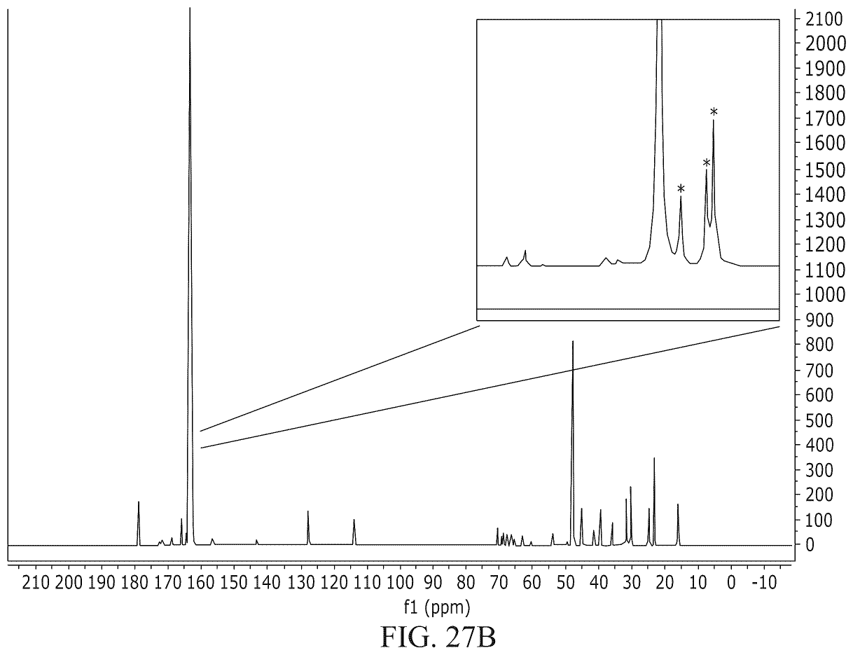

The results shown in FIG. 27A and FIG. 27B depict NMR of polymers. FIG. 27A depicts 1H NMR of the indicated polymers in the region characteristic of N-formyl (RR′N—CHO) substitution. FIG. 27B depicts ¹³C NMR of hDD90-118 prepared in the presence of 10% ¹³C-enriched N,N-dimethylformamide, under otherwise identical conditions to those used elsewhere in this document. All the ¹³C-enriched peaks (marked with asterisks) are assigned to N-formyl (N—CHO) groups in the 8-8.15 ppm range, unexpected for the previously reported composition.

### Example 20—Mass Ratio Testing of Polymer 76

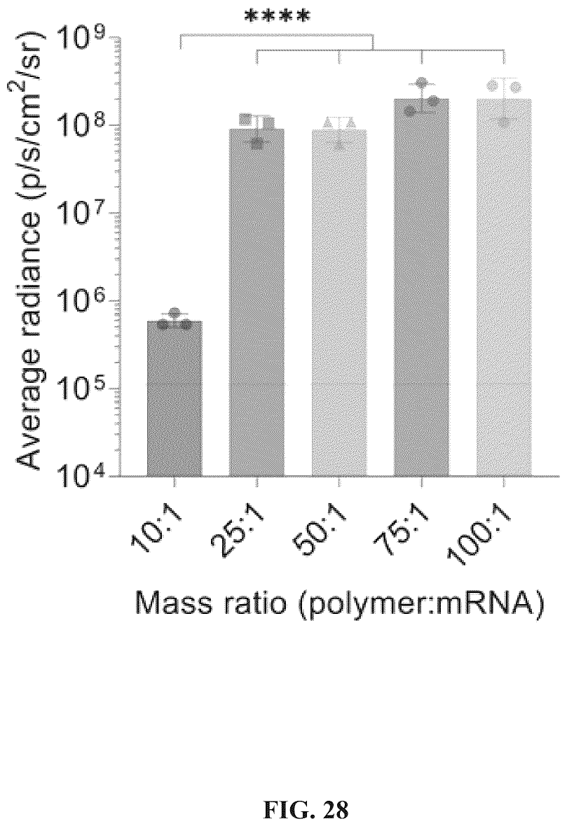

A range of mass ratios of polymer 76 to aNLuc mRNA at the indicated mass ratio was measured (FIG. 28). Polyplexes were nebulized at a dose of 25 μg/mouse and lungs were evaluated at 24 hours for aNLuc protein expression. Dotted line represents mean average radiance of the control group. Bars represent geometric mean±SD. n=3 mice per group. ****p<0.0001 by one-way ANOVA with Tukey's multiple comparisons on log-transformed data. The data represented in FIG. 28 indicate a range of molar mass ratios can be used.

### Example 21—Stability of Lyophilized and Fresh Polymer mRNA Compositions

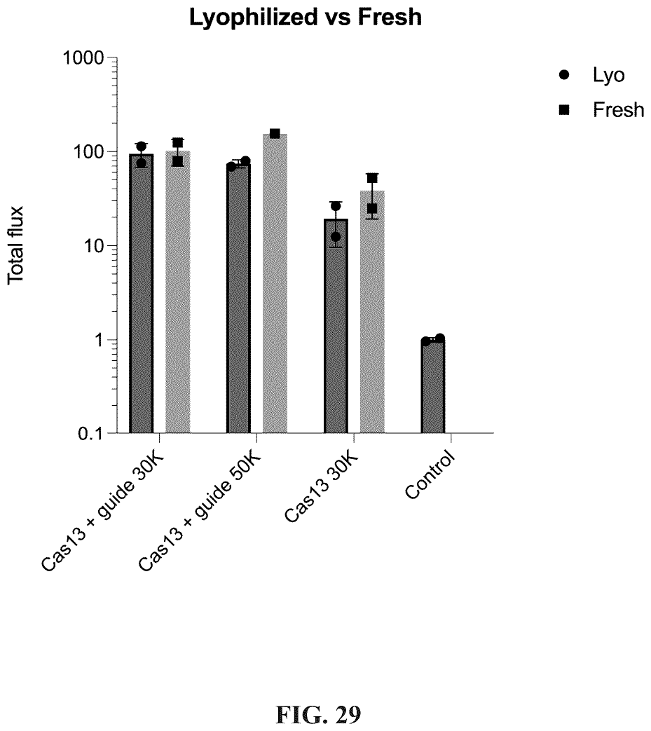

Stability of lyophilized and fresh polymer mRNA compositions were measured (FIG. 29). Nanoparticles were formulated with Cas 13-2A-NLuc mRNA with or without guide RNA and concentrated via centrifugal filters of indicated molecular weight cutoff. 5% of sucrose was added to the final volume after concentration, frozen in a cryogenic cooling chamber and lyophilized after 24 hours. Nanoparticles were delivered to mice using nebulizer. Luminescent reporter protein expression was measured (FIG. 29). The data represented in FIG. 29 indicate that no significant difference was observed between the freshly prepared (Fresh) and lyophilized (Lyo) solutions when assessing luminescent reporter protein expression.

### Example 22—Protein Expression in Hamster and Mouse Lung

Protein expression of mRNA molecules in hamster and mouse lungs when delivered with Polymer 76 via nebulizer were analyzed. The expression of mRNA was determined using an encoded beta-galactosidase (beta-gal) protein. Protein expression was then determined using immunofluorescence for the beta-gal protein. Protein expression was visualized using a Perkin Elmer Vectra Polaris slide scanning microscope.

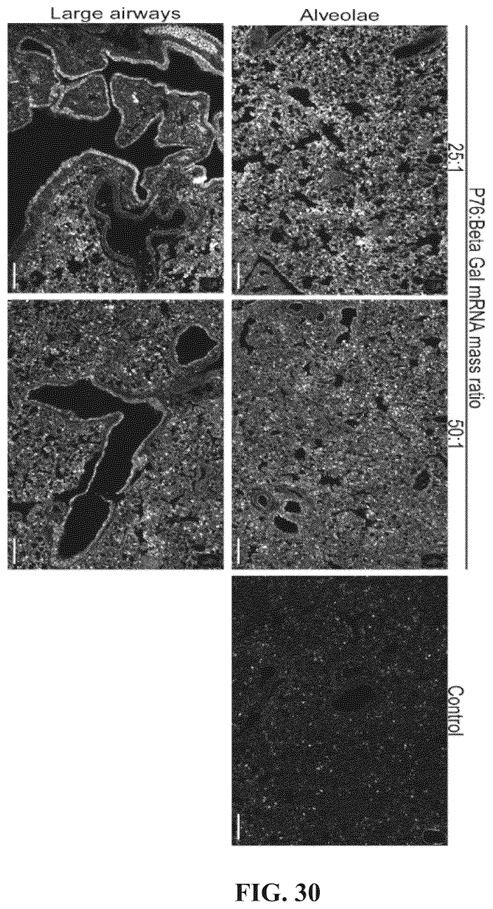

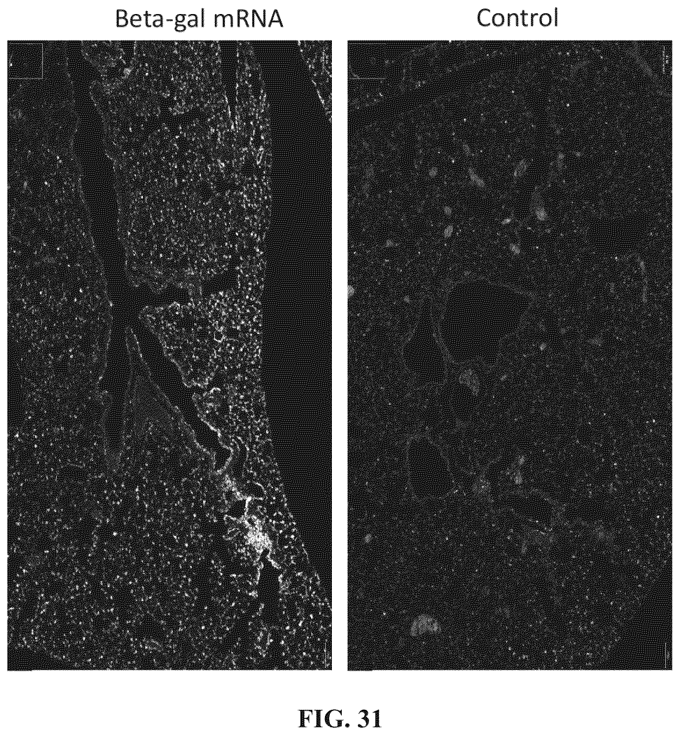

Polymer 76 was formulated with beta-gal mRNA. mRNA was first diluted to 1 mg/mL in sodium acetate buffer (pH 5.0). Polymers were dissolved in the same sodium acetate buffer at 50 mg/mL. mRNA and polymer solution was then combined at either 1:1 or 2:1 volume ratio and incubated for 10 minutes; the final concentration of mRNA and polymer is either 0.5 mg/mL and 25 mg/mL or 0.67 mg/mL and 16.7 mg/mL, respectively. The mRNA was delivered via nebulizer. Hamsters were dosed at 3 mg/kg and then euthanized and analyzed after 24 hours. The results are shown in FIG. 30, which demonstrates that Polymer 76 efficiently delivers beta-gal mRNA to the lungs of hamsters (particularly the large airways and aveolac). Mice were dosed at 2.5 mg/kg and then euthanized and analyzed after 24 hours. The results are shown in FIG. 31, which demonstrates that Polymer 76 efficiently delivers beta-gal mRNA to the lungs of mice.

### Example 23—Nebulization Technique Comparison

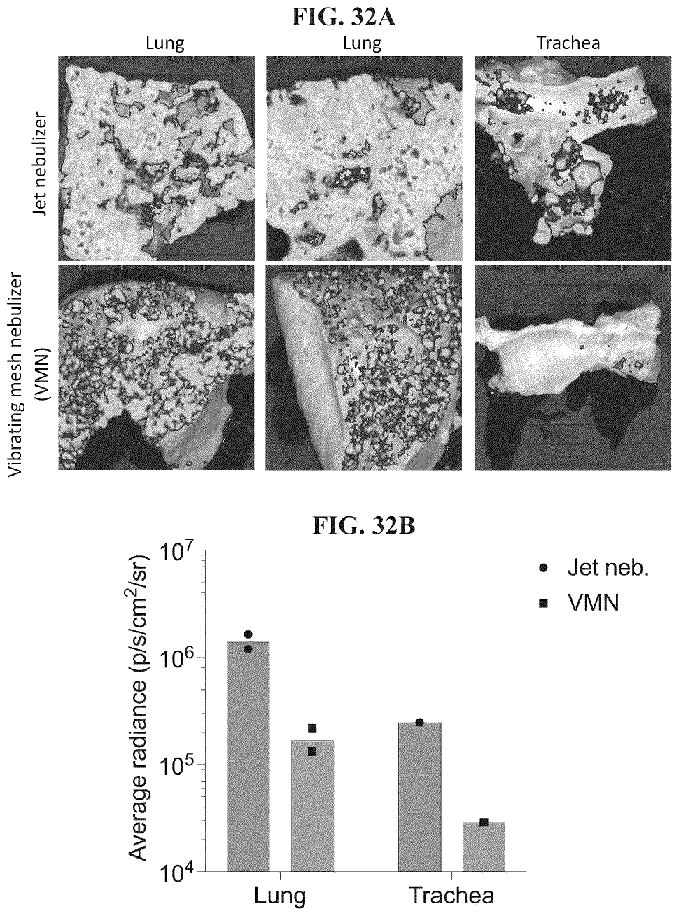

Two nebulization techniques, jet nebulizer and vibrating mesh nebulizer (VMN), were compared (FIGS. 32A-32B). The polymer was delivered to bovine with jet nebulizer or VMN driven by an air compressor at a dose of 0.07 mg/kg. Radiance was measured visualized (FIG. 32A) and quantified (FIG. 32B) in lung and trachea. The data represented in FIGS. 32A-32B indicate polymer 76 potently delivered mRNA to the lung via jet nebulization and vibrating mesh nebulizer is effective.

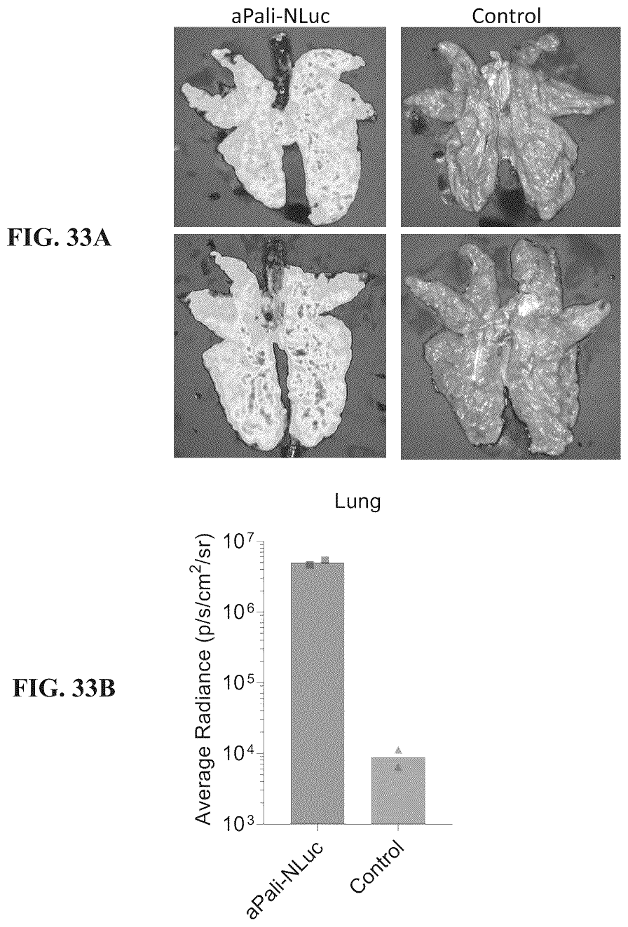

The jet nebulizer method was also tested in swine. A dose of 0.7 mg/kg polymer mRNA composition was delivered to the lung via jet nebulization. Radiance was measured visualized (FIG. 33A) and quantified (FIG. 33B) in lung. The data represented in FIGS. 33A-33B indicate polymer 76 effectively delivered mRNA to the lung via jet nebulization.

### Example 24—Toxicity Testing in Mice

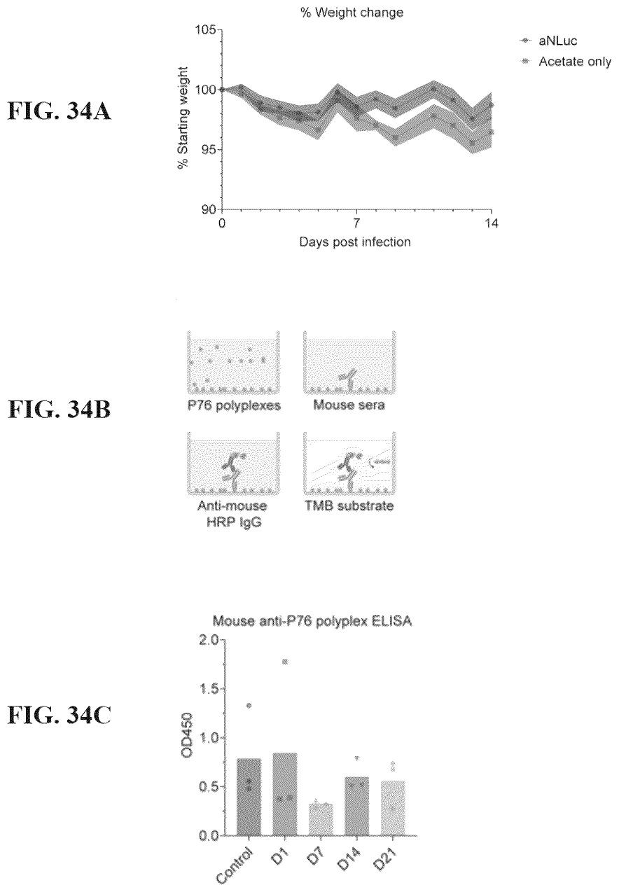

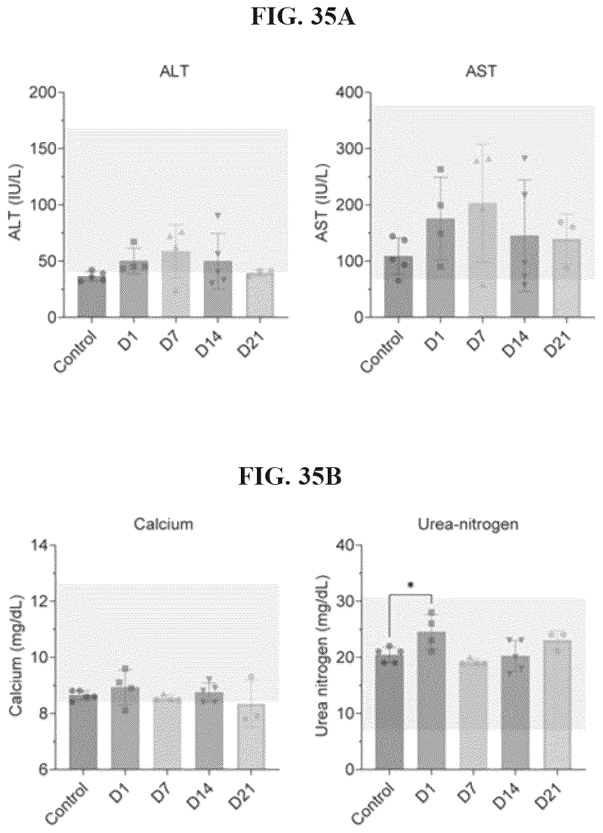

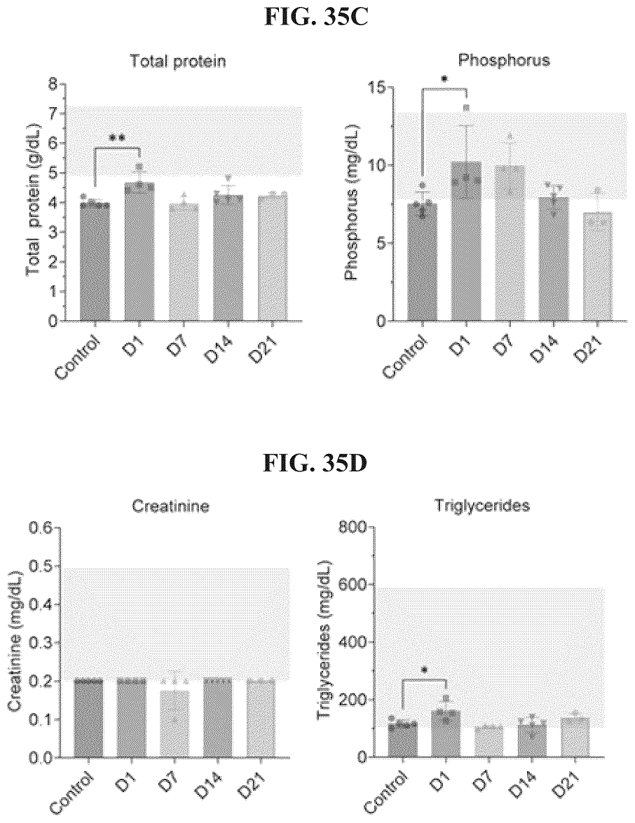

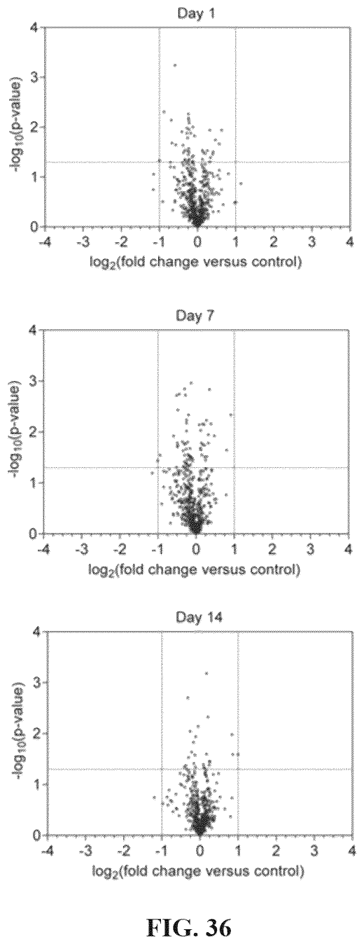

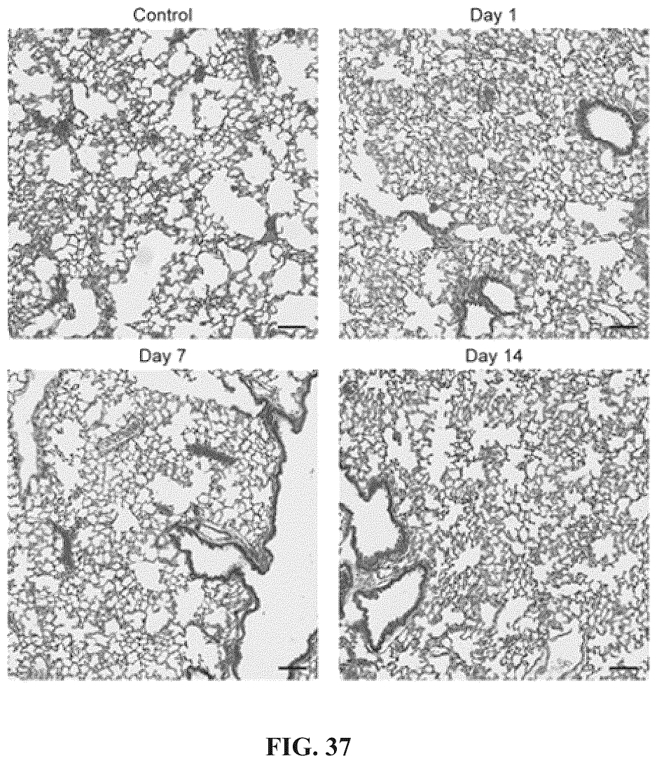

Toxicity of aNLuc mRNA delivered via polymer 76 was analyzed. Mice were treated with aNLuc mRNA delivered via polymer 76 at a dose of 1.25 mg/kg for analysis of weight changes (FIG. 34A), serum anti-P76 polyplex antibody levels (FIG. 34B-34C), complete blood chemistry (FIGS. 35A-36D), lung-tissue-level differential gene expression (FIG. 36), and histopathology (FIG. 37) over a 21-day period post exposure. Mice were sacrificed at days 1, 7, 14, and 21 for terminal blood and tissue collection. For cach time point, n=6 mice weights were measures (FIG. 34A). Antibodies against polymer 76 were analyzed to determine if an immunological response was induced (FIGS. 34B-34C). The results, shown in FIG. 35C, show no detectable levels of antibodies over background signal in serum out to 21 days. A blood chemistry panel was completed measuring alanine transaminase (ALT), aspartate transaminase (AST), Calcium, urca nitrogen, phosphorus, and triglycerides. At the indicated days, blood draws were performed on all mice (FIG. 35). The horizontal line represents p=0.05 (two-tailed t-test on log-transformed normalized data) and the vertical lines indicate fold changes of ±2. FIG. 36 depicts differential gene expression of 561 inflammatory genes as determined by NanoString in mice treated with aNLuc mRNA delivered via polymer 76 at a single 1.25 mg/kg dose at a range of timepoints. Lung tissue pathology was assessed by examining mouse lungs that were sectioned and stained with hematoxylin and cosin at day 1, 7, and 14 (FIG. 37). Images are representative lung sections from mice at the indicated time point. The scale bar is 100 mm and 2 mice were used per group. The data represented in FIGS. 34A-34C, in FIGS. 35A-35D, in FIG. 36, and in FIG. 37 indicate the toxicity of nebulized polymer 76 carrying mRNA cargo is minimal.

### Example 25—Capacity to Carry Mixed Cargo Lengths

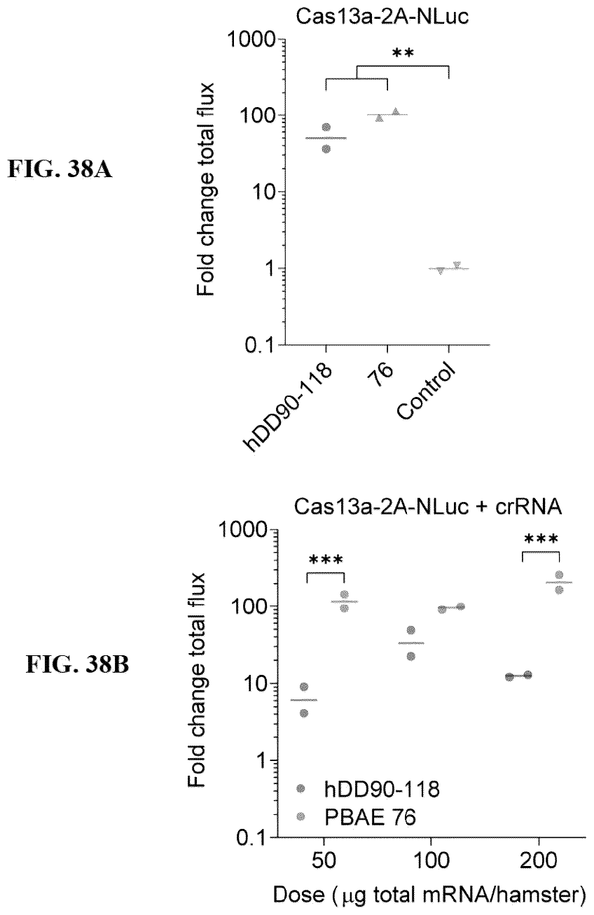

Mixed cargos of varying lengths were tested for use with polymer 76 (FIGS. 38A-38B). The Cas13a-NLuc reporter mRNA was delivered via nebulizer at 1.25 mg/kg to hamsters, finding that P76 delivery resulted in a 2.05-fold increase in signal (FIG. 38A). Hamster lung luminescence was measured and quantified 24 hours post transfection of 1.25 mg/kg of Cas13a-NLuc mRNA (FIG. 38A). The bars represent geometric mean±s.d. n=2. ** represents a p value<0.01 (one way ANOVA with Dunnett's multiple comparisons on log-transformed data). However, when a crRNA was delivered alongside the Cas13a-NLuc mRNA, the P76 formulation produced 19.1, 2.9, and 16.4 times more signal when delivered at 0.310, 0.625 and 1.250 mg kg⁻¹, respectively, compared with hDD90-118 (FIG. 38B). The increase in Cas 13-NLuc expression was not due to a difference in mRNA and crRNA binding between P76 and hDD90-118. Fold change total flux (FIG. 38B) show hamster lungs 24 hours after delivery of the indicated total dose of Cas13-NLus mRNA and crRNA delivered via hDD90-118 and polymer 76 (PBAE 76). Error bars represent geometric mean±s.d. n=2. ** represents a p value<0.01 (one way ANOVA with Šidák's multiple comparisons on log-transformed data). The data represented in FIGS. 38A-38B indicate that nebulized formula of polymer 76 is more efficient than hDD90-118 at delivering RNA cargo of different lengths.

### Example 26—Efficient Delivery of mRNA and crRNA Prevents SARS-COV-2 Infection in Hamster

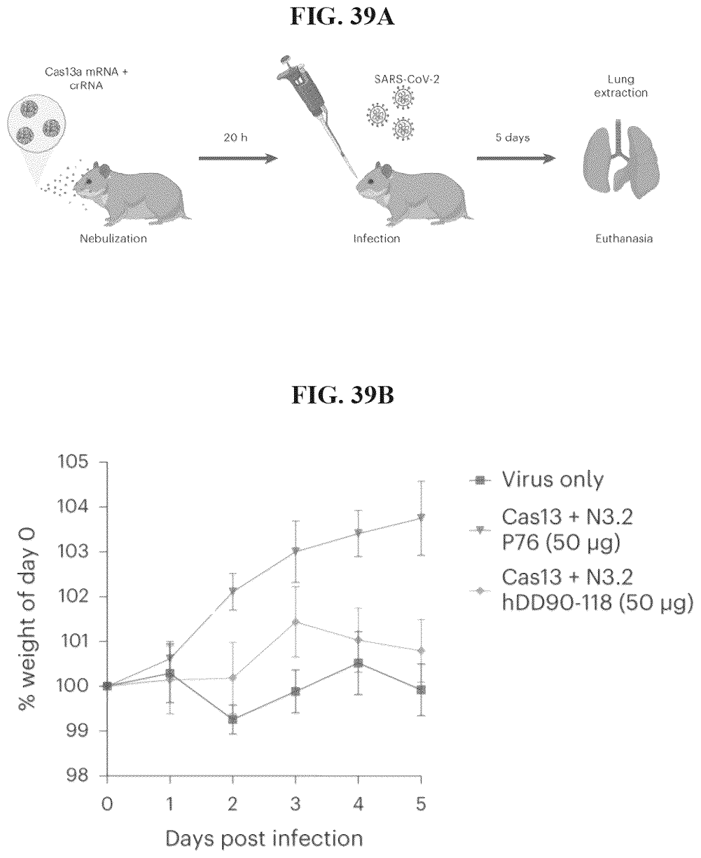

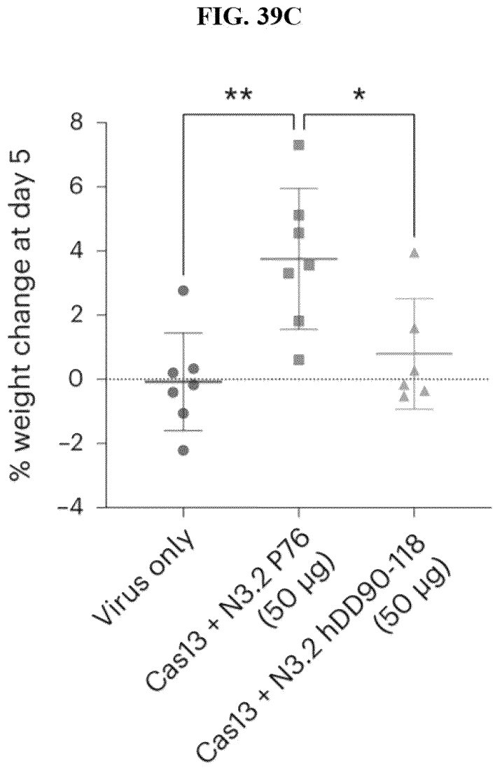

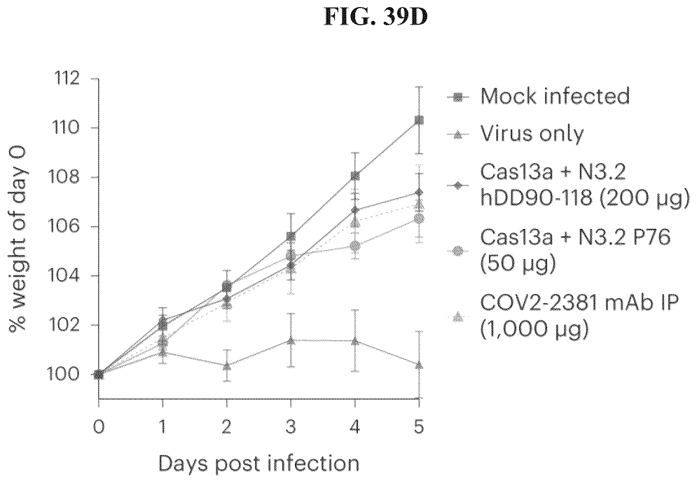

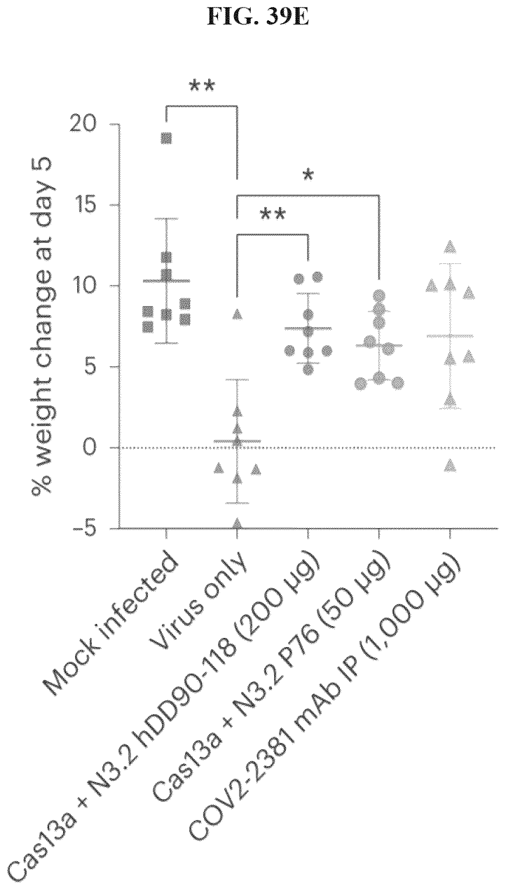

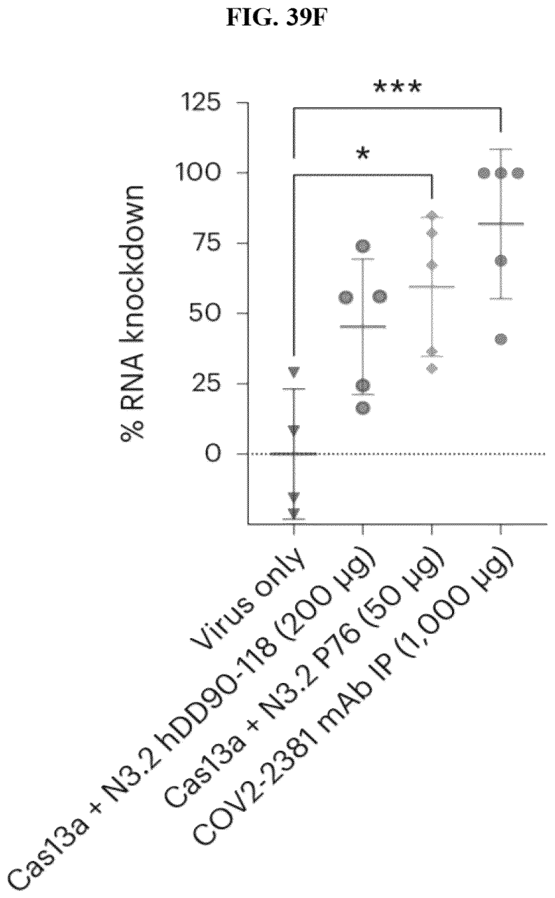

Hamsters were treated with LbuCas13a mRNA alongside our previously validated anti-SARS-COV-2 crRNA, N3.2, with a P76 or hDD90-118 formulation and intranasally infected 20 h later with 1,000 plaque-forming units (PFU) of the WA-1 strain of live SARS-COV-2 (FIG. 39A). Twenty hours later, the hamsters were intranasally inoculated with 10³ PFU of WA-1 SARS-CoV-2. The hamsters were euthanized on day 5 and the lungs were extracted and processed for viral load quantification. n=8 hamsters per group. Hamsters were weighed daily as a measure of general health, and when both P76 and hDD90-118 formulations were delivered at 0.5 mg kg⁻¹, only the P76 formulation prevented differential weight loss due to SARS-CoV-2 challenge over 5 days. Body weight was measured and depicted as percent normalized hamster weight over time (FIG. 39B). The symbols and error bars represent mean and per-cent weight±s.e.m., respectively. Percent hamster weight at day 5 post infection is depicted in FIG. 39C. The bars represent mean±s.d. The results depicted in FIG. 39C show the animals treated with a 0.5 mg/kg dose of P76-formulated Cas13 mRNA with N3.2 crRNA gained a significant amount of body weight by day 5 compared with both virus-only control group and 0.5 mg/kg hDD90-118 formulation. These data suggest that P76 is significantly more efficient at delivering the Cas13 mRNA and crRNA than hDD90-118, consistent with the luminescence data.

To further validate the potency of polymer 76 formulations, the same study was performed with the 0.5 mg/kg polymer 76 formulation and an increased dose of 2.0 mg/kg of the hDD90-118 formulation. These mRNA-based formulations were compared with one group of hamsters that was treated via intraperitoneal administration with 10 mg/kg of the potent neutralizing monoclonal antibody, COV2-2381, as a gold-standard control. One group of hamsters was mock infected, whereas another group was untreated and infected as a negative and positive control, respectively. It was found that the 0.5 mg/kg Cas13a mRNA dose using polymer 76 performed as well as both 2.0 mg/kg dose using hDD90-118 and 10.0 mg/kg dose of COV2-2381. with all the treated hamsters gaining 6.88% body weight on average over 5 day (FIG. 39D). The symbols and error bars represent mean and per-cent weight±s.e.m., respectively. Percent hamster weight at day 5 post infection is represented in FIG. 39E and shows that weight change at day 5 was only significantly improved by the hDD90-118 and P76 formulations and there was no significant difference among the treated groups. The bars represent mean±s.d. SARS-CoV-2 RNA knockdown in the lungs was only significantly reduced by 59.5% and 81.9% (FIG. 39F), compared with the virus-only group, at day 5 in hamsters treated with either polymer 76-formulated Cas13a or COV2-2381, respectively, but there was no significant difference between the treated groups. The bars represent mean±s.d. *p<0.05. **p<0.01, ***p<0.001 (one-way ANOVA with Dunnett's multiple comparisons). n=4-5 mice per group. The data represented in FIGS. 39A-39F demonstrate that the properties allowed for a four times lower dose in a SARS-CoV-2 challenge in a hamster model using P76-delivered Cas13a mRNA compared with the previously reported PBAE, with similar efficacy to the gold standard of systemic neutralizing antibody treatment.

### General Methods

### Nuclear Magnetic Resonance

Nuclear magnetic resonance (NMR) spectra were obtained on a Bruker DRX-500 or Bruker AV3 HD-700 instrument in CDCl₃ or CD₃OD. All 1H NMR experiments are reported in δ units, parts per million (ppm), and were measured relative to the signals for residual methanol (3.35 ppm) or chloroform (7.26 ppm).

### DLS Measurements

Polyplexes were prepared as described for in vivo usage with a final concentration of 0.5 mg/mL nucleic acid. Next, 10 μL of particles were then diluted into 990 μL of 100 mM sodium acetate, pH 5.0 in a sizing cuvette and analyzed using a Malvern Zetasizer Nano ZS. For zeta potential measurements, 0.4 mL of particles were diluted in 4.6 mL H₂O, pH 5.0, and loaded into a Malvern capillary for analysis.

### Molecular Dynamics Simulations

All-atom Molecular Dynamics (MD) was applied to investigate the interactions between the polymer and the mRNA. Only a monomer of the polymer being studied was built using the Molefacture plug-in of Visual Molecular Dynamics (VMD). From the crystal structure of RNase (7DIC), the nine-base-pair mRNA strand was selected and used to represent the mRNA utilized in experiments. One monomer was placed 50 Å away from the nine-base-pair mRNA from the crystal structure. The experimental molar ratios of the monomer to the number of base pairs of RNA is reflected in the systems, with one monomer to 11 basepairs. The mRNA and monomer were solvated in a TIP3P water box with 0.10M NaCl. The dimensions of the water box were 87 Å×87 Å×87 Å.

### CryoEM Characterization of the Particles

The mRNA and monomer were restrained for 1 ns with the water unrestrained to minimize and equilibrate system. Following this equilibration, the system was released and run for 1000 ns per replica, with three replicas total per system. In total, 6 μs of simulation was run between all systems and replicas. Production simulations were run on Georgia Tech's Hive cluster, using NAMD while initial equilibration were run using NAMD2. Each monomer was parameterized using CGenFF and the CHARMM36 force-field was used for water, ions, and RNA. All simulations were run at 310 K with constant pressure at 1 atm, enforced using Langevin dynamics with the damping coefficient at 1/ps and the Langevin piston method 40, and periodic boundary conditions. A timestep of 2 fs was utilized for the first 1 ns restraint, followed by a timestep of 4 fs with Hydrogen Mass Repartition (HMR) 41 for the full production runs. Van der waals interactions were cutoff at 12 Å with a switching function beginning at 11 Å. Long-range electrostatic interactions were calculated with the particle-mesh Ewald method 42, using a grid spacing of less than 1 Å.

Hydrogen bond analysis between specific selections of the monomer and the RNA was quantified using the Hydrogen Bond plug-in of VMD. Hydrogen bonds were defined using cut-offs 3.5 Å (D-A distance) and 30° (A-D-H angle). Contact frequency was calculated between the RNA and a selection of the monomer over the entire 1000 ns trajectories. The range of interactions was defined as within 4 Å.

### nCounter Analysis

Tissue was collected into tubes and flash frozen immediately on liquid nitrogen. RNA was extracted using Trizol as described above. RNA concentration and integrity was confirmed by spectrophotometer and BioAnalyzer (Agilent), respectively, before being analyzed on a NanoString nCounter using the mouse immunology panel according to manufacturer's instructions. Fold changes and p-values were calculated using the nSolver software (NanoString).

### Histology

H&E lung slides were examined by an ACVP board certified veterinary pathologist. For each animal, all lung lobes were used for analysis and affected microscopic fields were scored semiquantitatively as Grade 0 (None); Garde 1 (Minimal); Grade 2 (Mild); Grade 3 (Moderate) and Grade 4 (Severe). Scoring was performed based on these criteria: percent lung affected, type 2 pneumocyte hyperplasia, alveolar septal thickening, inflammatory infiltrates, and severity of broncho-interstitial pneumonia. An average and total lung score per group was calculated by combining scores from each criterion. No significant findings were observed across any of the assayed lungs. Digital images in FIG. 3j of H&E-stained slides were captured at 40× and 200× magnification with an Olympus BX43 microscope equipped with a digital camera (DP27, Olympus) using Cellsens® Standard 2.3 digital imaging software (Olympus). Images were captured by HistoWiz and analyzed by the same pathologist and in the same manner as above.

### In Situ Hybridization

At 4 hours post-delivery, animals were sacrificed, perfused with 1× PBS, and lungs were removed and incubated in 4% paraformaldehyde overnight at 4° C. Paraffin embedded 5 μm sections were prepared by HistoWiz. Delivered mRNA and endogenous mRNA was visualized using RNAscope Multiplex Fluorescent Reagent Kit v2 (Advanced Cell Diagnostics 323136) according to manufacturer's instructions. A custom probe set was designed against the synthetic aNLuc mRNA sequence (ACD 879571), aHCA-NLuc light chain mRNA sequence (ACD 1058321-C1), and Cas13a-NLuc mRNA sequence (ACD 1058341-C1). To distinguish lung airways, probes for secretoglobin (Scgb1a1, mouse: ACD 420351-C3, ferret: ACD 300040, macaque: ACD 1058211-C2) and Forkhead Box J1 (Foxj1, ferret: ACD 1058181-C3, and macaque: ACD 1058221-C3) were used. Lung tissue was scanned by the Emory Winship Cancer Tissue and Pathology core on a Perkin Elmer Vectra Polaris slide scanner.

### Equivalents and Scope

The invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Certain aspects of the invention are described in the following embodiments:
1. A polymer comprising a repeating monomeric unit, wherein the monomeric unit comprises one or more thioether moieties and one or more bisphenol moieties represented by:

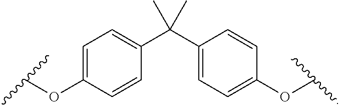

2. The polymer of embodiment 1, wherein the one or more thioether moieties are independently selected from

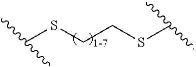

3. The polymer of embodiment 2, wherein the one or more thioether moieties are represented by

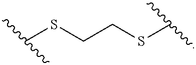

4. The polymer of any one of embodiments 1 to 3, wherein the monomeric unit comprises a thioether moiety represented by

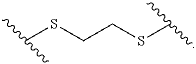

5. The polymer of any one of embodiments 1 to 4, wherein the monomeric unit comprises 1-5 bisphenol moieties, such as 2, 3, or 4 bisphenol moieties.
6. A polymer comprising a repeating monomeric unit, wherein the monomeric unit comprises one or more thioether moieties represented by

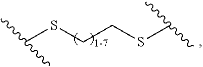

and one or more bisphenol ester moieties represented by

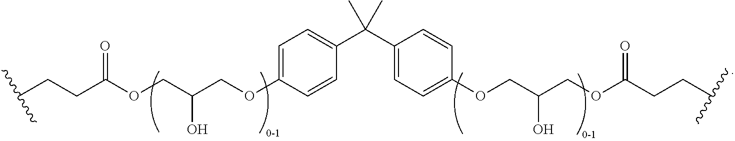

7. The polymer of embodiment 6, wherein the monomeric unit comprises a thioether moiety represented by

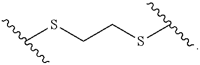 [SMILES: *SCCS*]

8. The polymer of embodiment 6 or 7, wherein the monomeric unit comprises 1-5 bisphenol ester moieties.
9. The polymer of embodiment 8, wherein the monomeric unit comprises 2, 3, or 4 bisphenol ester moieties.
10. The polymer of embodiment 9, wherein the monomeric unit comprises 3 bisphenol ester moieties.
11. The polymer of any one of embodiments 1 to 10, wherein a monomeric unit further comprises one or more amine groups.
12. The polymer of embodiment 11, wherein the one or more amine groups are selected from alkyldiamines.
13. The polymer of embodiment 12, wherein the alkyldiamine groups are independently selected from:

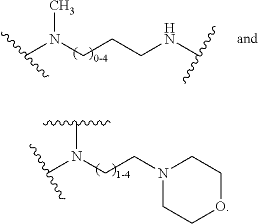

14. The polymer of embodiment 12 or 13, wherein the monomeric unit comprises 1, 2, or 3 alkyldiamines.
15. The polymer of embodiment 14, wherein the monomeric unit comprises 2 or 3 alkyldiamines.
16. The polymer of any one of the preceding embodiments, further comprising one or more terminal amine groups.
17. The polymer of embodiment 16, wherein the terminal amine group is selected from a terminal alkyldiamine.
18. The polymer of embodiment 17, wherein the terminal alkyldiamine is selected from:

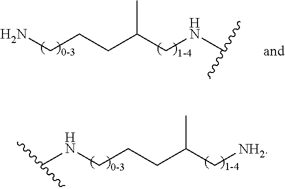

19. The polymer of embodiment 18, wherein the terminal alkyldiamine is selected from:

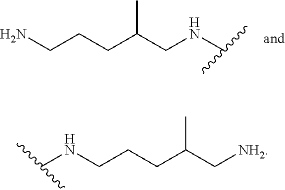

20. The polymer of any one of embodiments 11 to 19, wherein one or more of the amine groups and terminal amine groups are formylated.
21. The polymer of any one of embodiments 1 to 20, wherein the polymer comprises 5 to 100 monomeric units, such as 10 to 80 monomeric units, 5 to 50 monomeric units, 20 to 50 monomeric units, 30 to 60 monomeric units, or 40 to 50 monomeric units.
22. A polymer prepared by the method of contacting a compound of formula

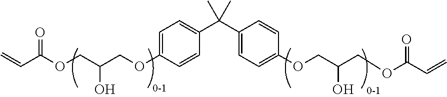

with a dithiol and one or more alkyldiamines in a solvent.
23. The polymer of embodiment 22, wherein the dithiol is selected from

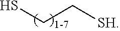

24. The polymer of embodiment 22 and 23, wherein the one or more alkyldiamines are selected from

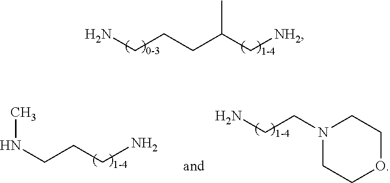

25. The polymer of any one of embodiments 22 to 24, wherein the solvent is polar aprotic.
26. The polymer of embodiments 25, wherein the polar aprotic solvent is N,N-dimethylformamide.
27. The polymer of any one of embodiments 22 to 26, wherein the mole ratio of dithiol to

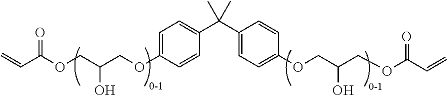

is about 1:20 to about 1:5, such as about 1:15 to about 1:8.
28. The polymer of embodiment 27, wherein the mole ratio of dithiol to

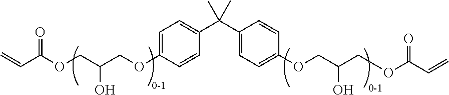

is about 1:10.
29. The polymer of any one of embodiments 22 to 28, wherein the mole ratio of the alkyldiamine to

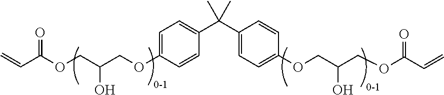

is about 1:5 to about 2:1.
30. The polymer of embodiment 1 or 21, comprising a monomeric unit represented by:

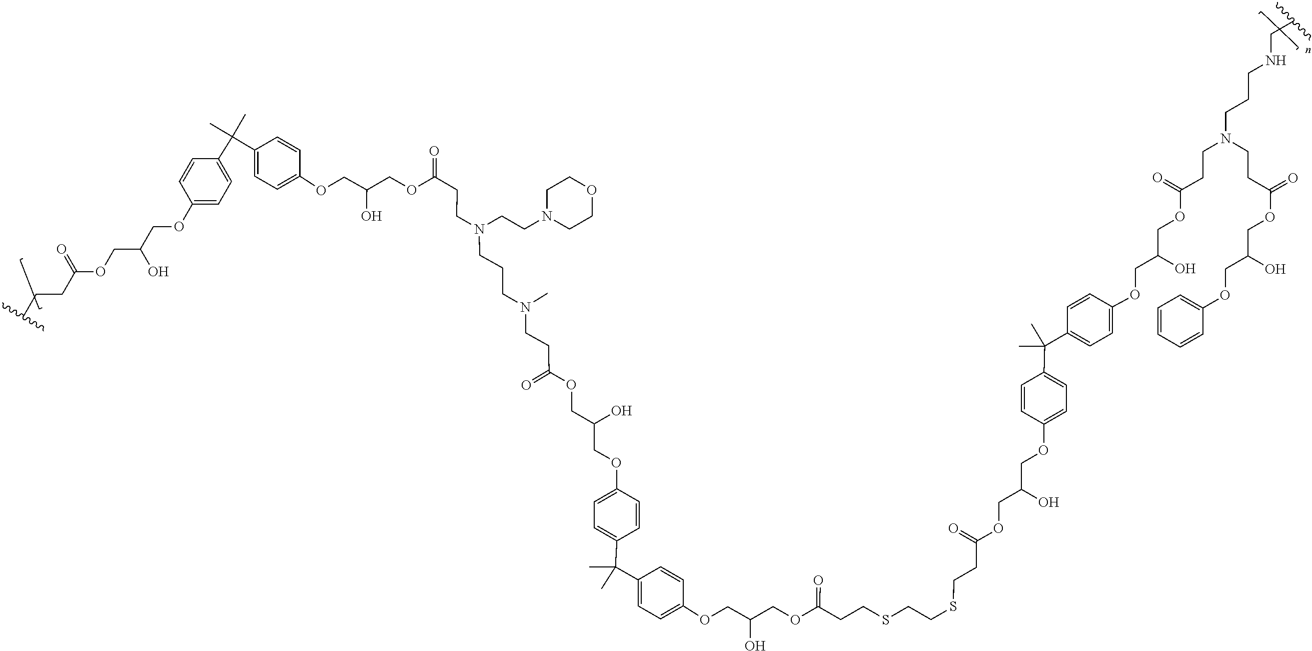

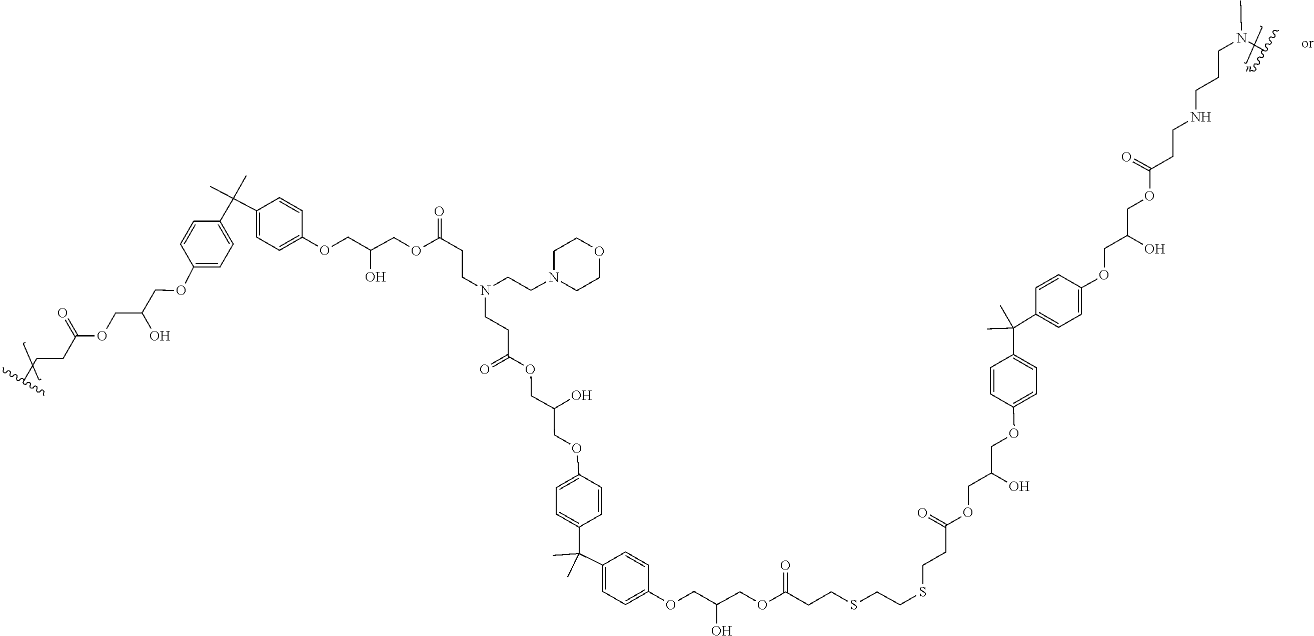

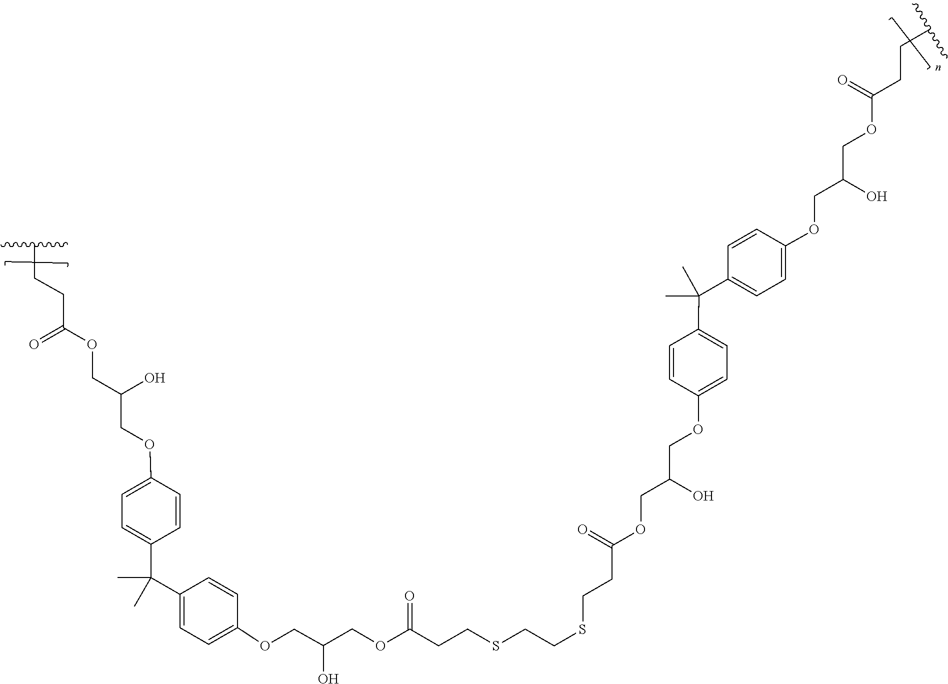

or a salt of any one thereof, wherein [ ]ₙ represents a monomeric unit of the polymer.
31. The polymer of embodiment 30, wherein the monomeric unit further comprises a formyl group on an amine moiety.
32. A complex comprising an oligonucleotide and a polymer, wherein the polymer is selected from the polymer of any one of embodiments 1 to 31.
33. The complex of embodiment 32, wherein the oligonucleotide comprises from 50 nt to 5500 nt.
34. The complex of embodiment 32 or 33, wherein the oligonucleotide is selected from mRNA and crRNA or a combination thereof.
35. The complex of any one of embodiments 32 to 34, wherein the mass ratio of oligonucleotide to polymer is from 1:10 to 1:100.
36. The complex of embodiment 35, wherein the mass ratio of oligonucleotide to polymer is from 1:25 to1: 100, from 1:25 to 1:75, from 1:30 to 1:70, or from 1:50 to 1:80.
37. The complex of any one of embodiments 32 to 36, wherein the complex comprises nanoparticles of the oligonucleotide and the polymer.
38. The complex of embodiment 37, wherein the nanoparticles have an average diameter of from 75 nm to 300 nm, 100 nm to 250 nm, 100 nm to 200 nm or 75 nm to 150 nm.
39. An aqueous suspension of particles comprising the complexes of any one of embodiments 32 to 38.
40. The aqueous suspension of embodiment 39, wherein the particles have a zeta potential of 45 mV or greater, such as 50 mV or greater, such as 50 to 80 mV, such as 55 to 65 mV.
41. The aqueous suspension of embodiment 39 or 40, wherein the suspension has a pH of 6 or less, or 5.5 or less, or 5 or less.
42. A method of delivering an oligonucleotide, comprising administering the aqueous suspension of embodiment 39, 40, or 41 to the lungs of a subject in need thereof.
43. The method of embodiment 42, wherein the aqueous suspension is delivered with a nebulizer.
44. The method of embodiment 43, wherein the nebulizer is a jet nebulizer or a vibrating mesh nebulizer.
45. The method of any one of embodiments 42 to 44, wherein the nebulized droplets have an average droplet diameter of from 2 μm to about 8 μm, such as from about 4 μm to about 7 μm, such as from about 4 μm to about 6 μm.
46. A method of preparing a polymer comprising contacting a compound of formula

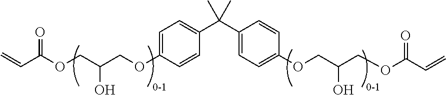

with a dithiol and one or more alkyldiamines in a solvent.
47. The method of embodiment 46, wherein the dithiol is selected from

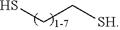

48. The method of embodiment 46 or 47, wherein the one or more alkyldiamines are selected from

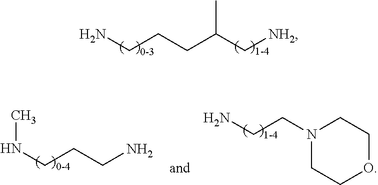

49. The method of any one of embodiments 46 to 48, wherein the solvent is polar aprotic.
50. The method of embodiments 49, wherein the polar aprotic solvent is N,N-dimethylformamide.
51. The method of any one of embodiments 46 to 50, wherein the mole ratio of dithiol to

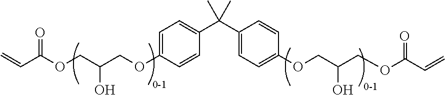

is about 1:20 to about 1:5.
52. The method of embodiment 51, wherein the mole ratio of dithiol to

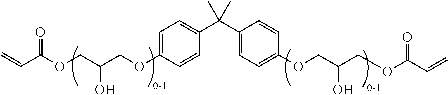

is about 1:10.
53. The method of any one of embodiments 46 to 52, wherein the mole ratio of the alkyldiamine to

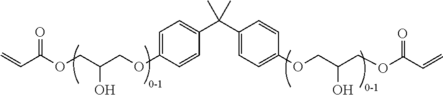

is about 1:5 to about 2:1.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-68. (canceled)

69. A polymer comprising one or more thioether moieties represented by 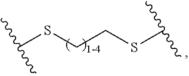
and one or more bisphenol ester moieties represented by

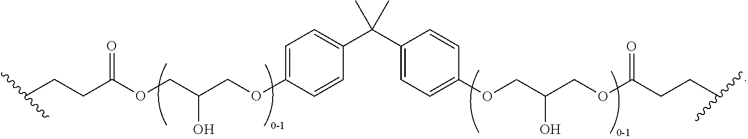

70. The polymer of claim 69, wherein the one or more thioether moieties are represented by

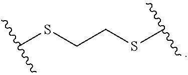 [SMILES: *SCCS*]

71. The polymer of claim 69, further comprising one or more amine groups.

72. The polymer of claim 71, wherein the one or more amine groups comprise one or more alkyldiamines.

73. The polymer of claim 72, wherein the one or more alkyldiamines are independently selected from:

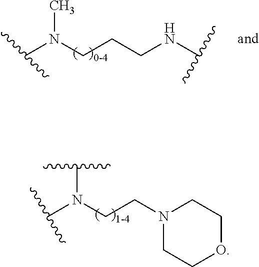

74. The polymer of claim 72, wherein the one or more amine groups further comprise one or more terminal amine groups.

75. The polymer of claim 74, wherein the one or more terminal amine groups are selected from one or more terminal alkyldiamines.

76. The polymer of claim 75, wherein the one or more terminal alkyldiamines are selected from:

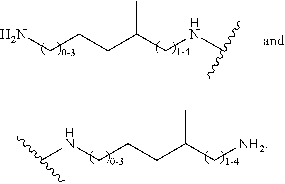

77. The polymer of claim 76, wherein the one or more terminal alkyldiamines are selected from:

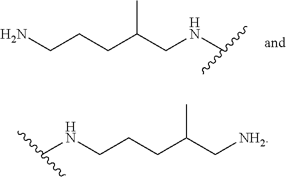

78. The polymer of claim 71, wherein one or more of the one or more amine groups are formylated.

79. A polymer prepared by a method comprising contacting a compound of formula 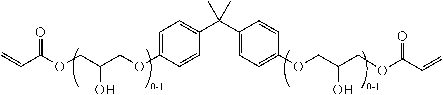
with a dithiol and one or more alkyldiamines in a solvent.

80. The polymer of claim 79, wherein the dithiol is selected from

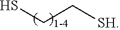

81. The polymer of claim 80, wherein the one or more alkyldiamines are selected from

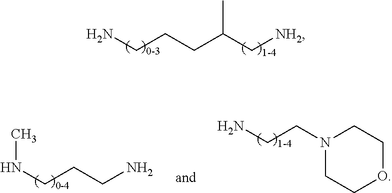

82. The polymer of claim 81, wherein the dithiol is 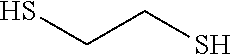
and the one or more alkyldiamines are

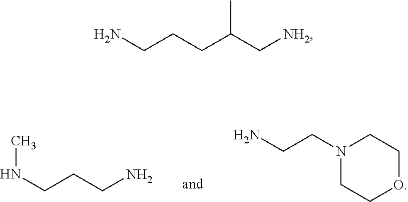

83. The polymer of claim 79, wherein the solvent comprises N,N-dimethylformamide.

84. The polymer of claim 81, wherein the mole ratio of the dithiol to the compound of formula 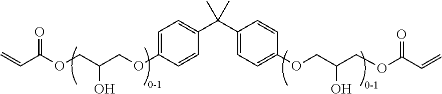
is about 1:20 to about 1:5; and
the mole ratio of each of the one or more the alkyldiamines to 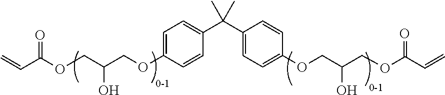
is independently about 1:5 to about 2:1.

85. A complex comprising an oligonucleotide and a polymer, wherein the polymer is selected from the polymer of claim 69.

86. The complex of claim 85, wherein the oligonucleotide comprises 50 nt to 5500 nt.

87. The complex of claim 85, wherein the oligonucleotide is selected from mRNA and crRNA or a combination thereof.

88. The complex of claim 85, wherein the mass ratio of the oligonucleotide to the polymer is from 1:10 to 1:100.

89. The complex of claim 85, wherein the complex comprises nanoparticles of the oligonucleotide and the polymer.

90. The complex of claim 89, wherein the nanoparticles have an average diameter of from about 100 nm to about 200 nm.

91. An aqueous suspension comprising the complex of claim 85.

92. The aqueous suspension of claim 91, wherein the suspension has a pH of 6 or less.

93. A method of delivering an oligonucleotide to a subject, comprising administering the aqueous suspension of claim 91 to the lungs of the subject.

94. The method of claim 94, wherein the aqueous suspension is delivered to the lungs of the subject with a nebulizer.
